(19) United States
(12) Patent Application Publication
(10) Pub. No.: US 2023/0083717 A1
(43) Pub. Date: Mar. 16, 2023

(54) METHODS AND COMPOSITIONS FOR PROMOTING ADIPOCYTE BEIGING

(71) Applicant: Flagship Pioneering Innovations VI, LLC, Cambridge, MA (US)

(72) Inventors: Fabian Alexander WOLF, Munich (DE); Angeliki CHALKIADAKI, Brookline, MA (US); Morag Helen STEWART, Boston, MA (US); Nicholas McCartney PLUGIS, Boston, MA (US)

(21) Appl. No.: 17/901,378

(22) Filed: Sep. 1, 2022

Related U.S. Application Data
(60) Provisional application No. 63/239,838, filed on Sep. 1, 2021.
(60) Provisional application No. 63/276,109, filed on Nov. 5, 2021.

(51) Int. Cl. C12N 5/077; C12N 5/00

(52) U.S. Cl. CPC C12N 5/0653; C12N 5/0006

(57) ABSTRACT

The present technology relates, inter alia, to perturbagens and methods for directing a change in the cell state of a progenitor cell. It also relates to methods for increasing a quantity of beige adipocytes, beige preadipocytes, and/or immediate progenitors thereof and/or the ratios thereof. Further, the present technology relates to methods for treating diseases or disorders characterized by, at least, abnormal numbers, ratios or bodily distributions of beige adipocytes, beige preadipocytes, white adipocytes, white preadipocytes, or immediate progenitors thereof, with respect to each other.

### CROSS-REFERENCE TO RELATED APPLICATIONS

This application claims priority to and benefit of U.S. Provisional Patent Application No. 63/239,838 filed on Sep. 1, 2021, and U.S. Provisional Patent Application No. 63/276,109 filed on Nov. 5, 2021, the contents of all of which are hereby incorporated by reference in their entireties.

### BACKGROUND

An understanding of cellular mechanisms relating to development of beige adipocytes, and their lineages, as well as methods and agents for directing changes in development of beige adipocytes, may be useful for treating diseases or disorders, characterized by abnormal numbers, ratios or bodily distributions of beige adipocytes, beige preadipocytes, white adipocytes, white preadipocytes, or immediate progenitors thereof with respect to each other.

### SUMMARY

Accordingly, in various aspects, the present technology provides methods for directing a change in the cell state of a progenitor cell and agents that are suitable for achieving such change. These agents are described herein as perturbagens. For instance, in various aspects, the present technology provides methods for generating beige adipocytes.

The present technology also provides methods for increasing the quantities and/or the ratios of beige adipocytes, beige preadipocytes, or immediate progenitors thereof. In some embodiments, the methods decrease the quantities and/or the ratios of white adipocytes and/or white preadipocytes. The present technology further provides methods for treating diseases or disorders characterized by, at least, abnormal ratios and/or abnormal numbers of beige adipocytes, and/or beige preadipocytes, and optionally, white adipocytes, and/or white preadipocytes, or immediate progenitors thereof. In various aspects, the cellular manipulations described herein are guided and/or mediated by gene signatures that reflect a cellular state and/or capacity for transitioning of a cell from one state to a different cellular state.

An aspect of the present technology relates to a method for directing a change in cell state of a progenitor cell comprising: contacting a population of cells comprising a progenitor cell with at least one perturbagen selected from Table 3, or a variant thereof, wherein the at least one perturbagen is capable of altering a gene signature in the progenitor cell; and wherein the progenitor cell is an adipocyte stem cell or a mesenchymal stem cell.

An aspect of the present technology relates to a method for directing a change in cell state of a progenitor cell, comprising: contacting a population of cells comprising a progenitor cell with at least one perturbagen capable of altering a gene signature in the progenitor cell, wherein altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1 and wherein the progenitor cell is an adipocyte stem cell or a mesenchymal stem cell.

An aspect of the present technology relates to a method for directing a change in cell state of a progenitor cell, comprising: contacting a population of cells comprising a progenitor cell with at least one perturbagen selected from Table 3, or a variant thereof, and capable of altering a gene signature in the progenitor cell; wherein altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1; and wherein the progenitor cell is an adipocyte stem cell or a mesenchymal stem cell.

In embodiments, altering the gene signature comprises a change in expression and/or activity of one or more genes in the progenitor cell of a network module designated in the network module column of Table 1.

In embodiments, the change in cell state provides an increase in the number of one or more of adipoblasts, Myf5− progenitor cells, beige preadipocytes, and/or beige adipocytes.

In embodiments, the increase in the number of adipoblasts, Myf5− progenitor cells, beige preadipocytes, and/or beige adipocytes is relative to the number of adipoblasts, Myf5− progenitor cells, beige preadipocytes, and/or beige adipocytes obtained from a population of progenitor cells that are not contacted with the at least one perturbagen and/or relative to the number of adipoblasts, Myf5− progenitor cells, beige preadipocytes, and/or beige adipocytes obtained from a population of progenitor cells prior to contacting with the at least one perturbagen.

In embodiments, the change in cell state provides an increase in the number of beige adipocytes. In embodiments, the increase in the number of beige adipocytes is relative to the number of beige adipocytes obtained from a population of progenitor cells that are not contacted with the at least one perturbagen and/or relative to the number of beige adipocytes obtained from a population of progenitor cells prior to contacting with the at least one perturbagen.

In embodiments, the number of mesenchymal stem cells is decreased. In embodiments, the number of mesenchymal stem cells is increased.

In embodiments, the increase in the number of mesenchymal stem cells is due in part to increased cell proliferation of the mesenchymal stem cells.

In embodiments, the increase in the number of mesenchymal stem cells is relative to the number of mesenchymal stem cells in a population of progenitor cells that are not contacted with the at least one perturbagen and/or relative to the number of mesenchymal stem cells in a population of progenitor cells prior to contacting with the at least one perturbagen.

In embodiments, the number of beige preadipocytes, and/or beige adipocytes is increased after contacting the population of progenitor cells comprising an adipocyte stem cell or mesenchymal stem cell with the at least one perturbagen. In embodiments, the at least one perturbagen is selected from Table 3, or a variant thereof, comprises at least 2, at least 3, at least 4, or at least 5 perturbagens selected from Table 3, or variants thereof.

In embodiments, the one or more genes are selected from the genes designated as an “up” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, or 55 genes selected from the genes designated as an “up” gene in the gene directionality column of Table 1.

In embodiments, the one or more genes are selected from MRPL12, MIF, BIRC5, CDK1, UBE2C, CISD1, TOMM70A, RRS1, TRAP1, DLD, GAPDH, CDK4, MRPS16, GALE, CAT, PFKL, CEBPA, SCP2, LPGAT1, NT5DC2, GNB5, LAP3, HSPA4, PPARG, HSPD1, NOLC1, SDHB, PGAM1, TIMM9, CCNB2, IFRD2, MPC2, STMN1, PARP1, UBE2A, GSTZ1, SCARB1, HADH, PEX11A, ETFB, HSD17B10, PXMP2, CIAPIN1, DNAJC15, FKBP4, SMARCA4, BZW2, VDAC1, ISOC1, CYCS, G3BP1, CD320, MYLK, ElF4EBP1, and/or PHGDH.

In embodiments, the one or more genes are selected from the genes designated as an “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, or 73 genes selected from the genes designated as an “down” gene in the gene directionality column of Table 1.

In embodiments, the one or more genes are selected from JUN, ID2, ZFP36, BAMBI, HERPUD1, NCK1, MYC, SQSTM1, NFKBIA, IER3, TIPARP, HES1, CYB561, HSD17B11, FYN, PLSCR1, CIRBP, FOS, NUCB2, S100A13, RSU1, ASAH1, TIMP2, COL1A1, MYL9, LOXL1, COL4A1, DNM1, MMP2, PTGS2, SSBP2, RTN2, VAT1, GM, PROS1, B4GAT1, PNP, DRAP1, PRSS23, IGFBP3, TPM1, ILK, SCRN1, FHL2, KDM5B, GADD45B, EGFR, GRN, SERPINE1, TMEM50A, CLTB, GNAI2, FKBP14, CALU,

STXBP1, HTRA1, UGDH, PLA2G4A, EXT1, CTSL, NENF, TGFBR2, EGR1, P4HA2, GNAS, APP, HSPA1A, ITGB5, EPB41L2, GSTM2, ICAM1, CEBPD, and/or CHIC2.

In embodiments, the contacting the population of progenitor cells occurs in vitro or ex vivo and/or in vivo in a subject.

In embodiments, the method increases or stimulates the beiging and/or browning of white adipose tissue

(WAT), optionally as compared to the beiging and/or browning of WAT in the absence of a perturbagen and/or prior to contacting with the at least one perturbagen.

An aspect of the present technology relates to a perturbagen for use in a method disclosed herein.

An aspect of the present technology relates to a method for promoting the formation of a beige adipocyte, or an immediate progenitor thereof, comprising: exposing a starting population of stem/progenitor cells comprising a non-lineage committed mesenchymal stem cell nor adipocyte stem cell to a perturbation having a perturbation signature that promotes the transition of the starting population of stem/progenitor cells into a beige adipocyte, wherein the perturbation signature comprises increased expression and/or activity of one or more of genes selected from the genes designated as an “up” gene in the gene directionality column of Table 1 and/or a decreased expression and/or activity in the non-lineage committed adipocyte stem cell or mesenchymal stem cell of one or more genes selected from the genes designated as an “down” gene in the gene directionality column of Table 1.

In embodiments, the perturbation signature comprises an increase in expression and/or activity of one or more genes in the progenitor cell of an activation of a network module designated in the network module column of Table 1.

An aspect of the present technology relates to a method of increasing a quantity of beige adipocytes, or immediate progenitors thereof, comprising: exposing a starting population of stem/progenitor cells comprising a non-lineage committed adipocyte stem cell or mesenchymal stem cell to a pharmaceutical composition that promotes the formation of lineage specific progenitor population selected from adipoblasts, Myf5− progenitor cells, beige preadipocytes, and/or beige adipocytes, the pharmaceutical composition promoting the transition of a primitive stem/progenitor population into the lineage specific progenitor population that has the capacity to differentiate into beige preadipocytes, and/or beige adipocytes or immediate progenitors thereof, wherein the pharmaceutical composition comprises at least one perturbagen selected from Table 3, or a variant thereof.

An aspect of the present technology relates to a method for treating a disease or disorder characterized by an abnormal number of beige adipocytes, white adipocytes, beige preadipocytes, and/or white preadipocytes, comprising: (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

An aspect of the present technology relates to a method for treating a disease or disorder characterized by an increased number of white adipocytes, and/or white preadipocytes, comprising: (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

An aspect of the present technology relates to a method for treating a disease or disorder characterized by a decreased number of beige adipocytes, and/or beige preadipocytes, comprising: (a) administering to a patient in need thereof a therapeutically effective amount of at least one perturbagen selected from

Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

In embodiments, the disease or disorder is obesity, morbid obesity, morbid obesity prior to surgery, obesity linked inflammation, obesity linked gallbladder disease, obesity induced sleep apnea, hyperlipidemia, dyslipidemia, hypercholesterolemia, atherogenic dyslipidemia, fatty liver disease (FLD), nonalcoholic fatty liver disease (NAFLD), Non-Alcoholic Steatohepatitis (NASH), Prader-Willi Syndrome, insulin resistance, glucose intolerance, pre-diabetes, increased fasting glucose, diabetes mellitus, type 2 diabetes mellitus, inflammation, vascular inflammation, hypertension, endothelial dysfunction, atherosclerosis, arteriosclerosis, coronary heart disease, peripheral artery disease, stroke, or microvascular disease, arterial remodelling, cardiovascular disease (CVD), atherosclerotic cardiovascular disease, metabolic syndrome or a combination of any two or more thereof. obesity, hyperlipidemia, NAFLD, Type II Diabetes, inflammation, hypertension, cardiovascular disease, a metabolic disorder, excess body weight, atheromatous disease, β-cell dysfunction, heart disease, hyperglycemia, impaired glucose tolerance, an inflammatory disorder, latent autoimmune diabetes (LADA), nephropathy, neuropathy, retinopathy, Syndrome X, and/or type 1 diabetes.

In embodiments, the method alleviates one or more symptoms selected from the group of abdominal obesity, a body mass index (BMI) of 30 or higher, a blood triglyceride level of 150 or higher, a blood HDL level of less than 40 or higher in men, a blood HDL level of 50 or higher in women, a blood pressure of 130/85 or higher, a fasting blood sugar of 110 or higher, hyperlipidemia, dyslipidemia, hypercholesterolemia, atherogenic dyslipidemia insulin resistance, glucose intolerance, pre-diabetes, increased fasting glucose, diabetes mellitus, and a combination of any two or more thereof.

An aspect of the present technology relates to a method for selecting a subject a method disclosed herein, comprising: obtaining from a subject having the disease or disorder a sample of cells comprising a non-lineage committed adipocyte stem cell or mesenchymal stem cell; and contacting the sample of cells with least one perturbagen selected from Table 3 or a variant thereof, wherein the at least one perturbagen alters a gene signature in the sample of cells, the subject is selected as a patient.

An aspect of the present technology relates to a method for selecting a subject for a method disclosed herein, comprising: obtaining from a subject having the disease or disorder a sample of cells comprising a non-lineage committed adipocyte stem cell or mesenchymal stem cell; and contacting the sample of cells with at least one perturbagen capable of altering a gene signature in a non-lineage committed adipocyte stem cell or mesenchymal stem cell, wherein the at least one perturbagen increases in the sample of cells the expression and/or activity of one or more genes selected from the genes designated as an “up” gene in the gene directionality column of Table 1, and/or decreases in the sample of cells the expression and/or activity of one or more genes selected from the genes designated as an “down” gene in the gene directionality column of Table 1, the subject is selected as a patient.

An aspect of the present technology relates to a method for selecting a subject for method disclosed herein, comprising: obtaining from a subject having the disease or disorder a sample of cells comprising a non-lineage committed adipocyte stem cell or mesenchymal stem cell; and contacting the sample of cells with least one perturbagen selected from Table 3 or a variant thereof, wherein the at least one perturbagen increases in the sample of cells the expression and/or activity of one or more genes selected from the genes designated as an “up” gene in the gene directionality column of Table 1, and/or decreases in the sample of cells the expression and/or activity of one or more genes selected from the genes designated as an “down” gene in the gene directionality column of Table 1, the subject is selected as a patient.

In embodiments, the perturbation signature comprises an increase in expression and/or activity of one or more genes in the progenitor cell of an activation of a network module designated in the network module column of Table 1.

In embodiments, the one or more genes are selected from MRPL12, MIF, BIRC5, CDK1, UBE2C, CISD1, TOMM70A, RRS1, TRAP1, DLD, GAPDH, CDK4, MRPS16, GALE, CAT, PFKL, CEBPA, SCP2, LPGAT1, NT5DC2, GNB5, LAP3, HSPA4, PPARG, HSPD1, NOLC1, SDHB, PGAM1, TIMM9, CCNB2, IFRD2, MPC2, STMN1, PARP1, UBE2A, GSTZ1, SCARB1, HADH, PEX11A, ETFB, HSD17B10, PXMP2, CIAPIN1, DNAJC15, FKBP4, SMARCA4, BZW2, VDAC1, ISOC1, CYCS, G3BP1, CD320, MYLK, EIF4EBP1, and/or PHGDH.

In embodiments, the one or more genes are selected from the genes designated as an “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, or 55 genes selected from the genes designated as an “down” gene in the gene directionality column of Table 1.

In embodiments, the one or more genes are selected from JUN, ID2, ZFP36, BAMBI, HERPUD1, NCK1, MYC, SQSTM1, NFKBIA, IER3, TIPARP, HES1, CYB561, HSD17B11, FYN, PLSCR1, CIRBP, FOS, NUCB2, S100A13, RSU1, ASAH1, TIMP2, COL1A1, MYL9, LOXL1, COL4A1, DNM1, MMP2, PTGS2, SSBP2, RTN2, VAT1, GM, PROS1, B4GAT1, PNP, DRAP1, PRSS23, IGFBP3, TPM1, ILK, SCRN1, FHL2, KDM5B, GADD45B, EGFR, GRN, SERPINE1, TMEM50A, CLTB, GNAI2, FKBP14, CALU, STXBP1, HTRA1, UGDH, PLA2G4A, EXT1, CTSL, NENF, TGFBR2, EGR1, P4HA2, GNAS, APP, HSPA1A, ITGB5, EPB41L2, GSTM2, ICAM1, CEBPD, and/or CHIC2.

In embodiments, the at least one perturbagen is selected from Table 3, or a variant thereof, comprises at least 2, at least 3, at least 4, or at least 5 perturbagens selected from Table 3, or variants thereof.

An aspect of the present technology relates to a method of identifying a candidate perturbation for promoting the transition of a starting population of progenitor cells comprising an adipocyte stem cell or a mesenchymal stem cell into beige adipocytes or immediate progenitors thereof, the method comprising: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into beige adipocytes or immediate progenitors thereof following exposure of the population of cells to the perturbation; and identifying the perturbation as a candidate perturbation for promoting the transition of a population of progenitor cells into beige adipocytes or immediate progenitors thereof based on the perturbation signature, wherein the perturbation signature is an increase in expression and/or activity in the progenitor cell of one or more genes selected from the genes designated as an “up” gene in the gene directionality column of Table 1, and/or a decrease in expression and/or activity in the progenitor cell of one or more genes selected from the genes designated as an “down” gene in the gene directionality column of Table 1.

In embodiments, the perturbation signature is an increase in expression and/or activity in the progenitor cell of an activation of a network module designated in the network module column of Table 1.

An aspect of the present technology relates to a method for making a therapeutic agent for a disease or disorder selected from obesity, hyperlipidemia, NAFLD, Type II Diabetes, inflammation, hypertension, and/or cardiovascular disease, comprising:(a) identifying a candidate perturbation according to the methods disclosed herein and (b) formulating the candidate perturbation as a therapeutic agent for the treatment of the disease or disorder.

An aspect of the present technology relates to a method for directing a change in cell state of a progenitor cell, comprising: contacting a population of cells comprising a progenitor cell with at least one perturbagen capable of altering a gene signature in the progenitor cell, wherein altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1; wherein the progenitor cell is an adipocyte stem cell or a mesenchymal stem cell; and the change in cell state provides an increase in the number of one or more of adipoblasts, Myf5− progenitor cells, beige preadipocytes, and/or beige adipocytes.

In embodiments, the increase in the number of adipoblasts, Myf5− progenitor cells, beige preadipocytes, and/or beige adipocytes is relative to the number of adipoblasts, Myf5− progenitor cells, beige preadipocytes, and/or beige adipocytes obtained from a population of progenitor cells that are not contacted with the at least one perturbagen

In embodiments, the increase in the number of adipoblasts, Myf5− progenitor cells, beige preadipocytes, and/or beige adipocytes is relative to the number of adipoblasts, Myf5− progenitor cells, beige preadipocytes, and/or beige adipocytes obtained from a population of progenitor cells prior to contacting with the at least one perturbagen

In embodiments, the at least one perturbagen is selected from Table 3, or a variant thereof.

In embodiments, the at least one perturbagen comprises at least 2, at least 3, at least 4, or at least 5 perturbagens selected from Table 3, or variants thereof.

In embodiments, the method provides an increase in expression and/or activity of uncoupling protein 1 (UCP1).

In embodiments, the change in cell state provides an increase in the number of beige adipocytes.

In embodiments, the one or more genes are selected from MRPL12, MIF, BIRC5, CDK1, UBE2C, CISD1, TOMM70A, RRS1, TRAP1, DLD, GAPDH, CDK4, MRPS16, GALE, CAT, PFKL, CEBPA, SCP2, LPGAT1, NT5DC2, GNB5, LAP3, HSPA4, PPARG, HSPD1, NOLC1, SDHB, PGAM1, TIMM9, CCNB2, IFRD2, MPC2, STMN1, PARP1, UBE2A, GSTZ1, SCARB1, HADH, PEX11A, ETFB, HSD17B10, PXMP2, CIAPIN1, DNAJC15, FKBP4, SMARCA4, BZW2, VDAC1, ISOC1, CYCS, G3BP1, CD320, MYLK, EIF4EBP1, and/or PHGDH.

In embodiments, the one or more genes are selected from JUN, ID2, ZFP36, BAMBI, HERPUD1, NCK1, MYC, SQSTM1, NFKBIA, IER3, TIPARP, HES1, CYB561, HSD17B11, FYN, PLSCR1, CIRBP, FOS, NUCB2, S100A13, RSU1, ASAH1, TIMP2, COL1A1, MYL9, LOXL1, COL4A1, DNM1, MMP2, PTGS2, SSBP2, RTN2, VAT1, GM, PROS1, B4GAT1, PNP, DRAP1, PRSS23, IGFBP3, TPM1, ILK, SCRN1, FHL2, KDM5B, GADD45B, EGFR, GRN, SERPINE1, TMEM50A, CLTB, GNAI2, FKBP14, CALU, STXBP1, HTRA1, UGDH, PLA2G4A, EXT1, CTSL, NENF, TGFBR2, EGR1, P4HA2, GNAS, APP, HSPA1A, ITGB5, EPB41L2, GSTM2, ICAM1, CEBPD, and/or CHIC2.

Any aspect or embodiment disclosed herein can be combined with any other aspect or embodiment as disclosed herein.

### BRIEF DESCRIPTION OF THE DRAWINGS

FIG. 1 shows a t-distributed stochastic neighbor embedding (t-SNE) plot illustrating the predictions that drive the transition of cells from adipocyte stem cells to beige pre-adipocytes. Clusters are highlighted in different shades. ASC, adipocyte stem cells; MAC, macrophages; VEC, vascular endothelial cells; NKT, natural killer T cells; FB, fibroblasts; diff. ASC, differentiating adipocyte stem cells; VEC, vascular endothelial cells.

FIG. 2A shows a workflow scheme of differentiating 3T3L1 cells into white, beige or in the presence of predicted compounds.

FIG. 2B shows the results of an assay performed as shown in FIG. 2A. The results for the compounds that induced significant increase in the number of UCP1-positive cells, compared to DMSO-treated cells, in three independent experiments, are shown. Data bars from left to right are: DMSO, WHITE, BEIGE, Perturbagen 1 (0.1 μM), Perturbagen 1 (1 μM), Perturbagen 2 (1 μM), Perturbagen 2 (10 μM), Perturbagen 3 (0.1 μM), Perturbagen 3 (1 μM), Perturbagen 4 (0.1 μM), Perturbagen 4 (1 μM), Perturbagen 5 (1 μM), and Perturbagen 5 (10 μM).

FIGS. 3A-3B show that CL 316234 treated mice showed increased expression of UCP1 compared to vehicle only-treated mice (control). FIG. 3A shows the immunohistochemistry images of sections of epididymal (visceral) or inguinal (subcutaneous) fat from mice treated with CL 316234 or vehicle (control). Subcutaneous and visceral adipose tissues were isolated from mice dosed i.p. with vehicle (control) or CL 316234 for 7 days, fixed and stained for UCP1. FIG. 3B shows western blot analysis of protein extracts prepared from adipose tissues of mice dosed with mice treated with vehicle alone (−) or CL 316234 (+). UCP1 protein was detected using a specific antibody (See Table 4), and tubulin expression served as a loading control.

FIG. 4 shows a non-limiting, exemplary lineage of beige, white, and brown adipocytes from stem cells.

FIG. 5 depicts the plasma concentration of Perturbagen 6 at different time points from 0 to 24 h after IV administration.

FIG. 6 depicts the pharmacokinetic properties calculated from data obtained following subcutaneous administration of Perturbagen 6 in male C57BL/6 mice. FIG. 7 depicts the pharmacokinetic properties calculated from data obtained following oral administration of Perturbagen 6 in male C57BL/6 mice.

FIG. 8 depicts the plasma Perturbagen 6 concentration at different time points from 0 to 24 h after IP administration.

FIG. 9 depicts experimental data demonstrating that Perturbagen 6 reduced weight gain induced by 9 weeks of high fat diet. ****p<0.001, n=11-12.

FIGS. 10A-10C depict experimental data demonstrating that Perturbagen 6 treatment reduced inguinal (FIG. 10A) and epididymal fat (Figure. 10B) but not gastrocnemius muscle mass (FIG. 10C). ****p<0.001, n=11-12.

FIGS. 11A-11C depict experimental data demonstrating that Perturbagen 6 altered the adipocyte size distribution (FIG. 11A) in inguinal fat (FIG. 11B-FIG. 11C). *p<0.05, n=3.

FIGS. 12A-12D depict experimental data demonstrating that Perturbagen 6 treatment reduced plasma leptin (FIG. 12A), fasting glucose (FIG. 12B), and insulin (FIG. 12C), and improved HOMA-IR (FIG. 12D). *p<0.05, **p<0.01,***p<0.001****p<0.0001, n=6-12.

FIGS. 13A-13B depict experimental data demonstrating that Perturbagen 6 dose dependently reduced body weight gain in DIO mice. ***p<0.001, ****p<0.0001. n=8-10 per groups.

FIG. 14 depicts experimental data demonstrating that Perturbagen 6 dose dependently reduces food intake in DIO mice. *p<0.05, ****p<0.0001. n=8-10 per group.

FIGS. 15A-15B depict experimental data demonstrating that Perturbagen 6 dose dependently improved glucose tolerance test in DIO mice.

FIGS. 16A-16C depict experimental data demonstrating that the in-vivo dataset captures white and beige adipocyte cell states. FIG. 16A depicts a single cell manifold from inguinal adipose tissue.

FIG. 16B depicts the characterization of beige and white adipocytes population in inguinal adipose tissue based on UCP1 expression. FIG. 16C depicts modeling of dynamic transition of cell states induced by cold and drug treatments.

FIGS. 17A-17D depict experimental data demonstrating that in-vitro human adipocytes treated with pioglitazone capture white and beige adipocyte cell states. FIG. 17A depicts a schematic of the experimental design. FIG. 17B depicts the change in adipocyte population induced by pioglitazone treatment. FIG. 17C depicts an analysis of UCP1 and CIDEA expression. FIG. 17D depicts the characterization of beige and white adipocytes population based on UCP1 expression induced by pioglitazone.

FIGS. 18A-18B depict experimental data showing an example of in silico model of cellular behavior. FIG. 18A depicts a non-limiting example of map integrating disease biology with supporting visualizations to enable cross-functional insight generation. FIG. 18B depicts a table demonstrating similarities in cell behavior modules defining cell transition between in vitro and in vivo preclinical models.

FIG. 19 depicts experimental data showing the use of a machine learning platform to identify small molecule likely to induce beigeing of white adipocytes. The machine learning platform provides a new paradigm to identify new chemistry to induce targeted cell behavior.

FIGS. 20A-20E depict experimental data showing the identification of Perturbagen-1317 as a beigeing agent. FIG. 20A depicts a graph of experimental data showing that Perturbagen-1317 dose-dependently increases oxygen consumption rate (OCR) in human adipocytes. FIG. 20B depicts agraph of experimental data showing dose dependent induction of UCP1 gene expression in human adipocytes. Treatment of diet-induced obese mice with Perturbagen-1317 was found to reduce weight gain (FIG. 20C), improve fasting glucose (FIG. 20D), and reduced feeding efficiency (FIG. 20E).

### DETAILED DESCRIPTION

The present technology is based, in part, on the discovery that cells of beige adipocytes, and their progenitors can be characterized by specific gene signatures. Additionally, the present technology is based on the discovery that certain active agents (i.e., perturbagens) can alter these specific gene signatures. Such alteration is associated with the acquisition of specific cell states by cells of beige adipocyte lineages. These perturbagens are, in some instance, useful as therapeutics and derive benefit by directing the progenitor cells towards beige adipocyte states, and are thereby useful in the methods of treatment of obesity, hyperlipidemia, NAFLD, Type II Diabetes, inflammation, hypertension, cardiovascular disease, or related diseases or disorders described infra, in subjects in need thereof.

Adipose tissue can range from 4% to >40% of total body weight. Mammals contain white adipose tissue (WAT), and the brown adipose tissue (BAT). WAT is typically dispersed throughout the body in subcutaneous and intra-abdominal (or visceral) locations. Intra-abdominal fat includes retroperitoneal, omental, mesenteric, epicardial, and gonadal fat. WAT generally represents as much as 20% of the body weight of normal adult human and primarily acts as a storage site for triglycerides, conserving excess calories for use in times of scarcity. WAT comprises white adipocytes, which contribute to the whole body insulation and have endocrine functions including secretion of leptin, TNF-α, adiponectin, resistin, and other compounds related to the degree of obesity and insulin sensitivity. White adipocytes have a characteristic large lipid droplet that fills the cellular space, while the cellular structures (nuclei, mitochondria) are placed near the cellular membrane.

Brown adipose tissue (BAT) plays a crucial role in whole-body energy homeostasis through non-shivering thermogenesis. In infants, classical brown adipocytes (or brown fat) are located in the dedicated BAT depots, such as the interscapular regions, paravertebrally, axillary, and in perirenal space. Brown adipocytes are usually smaller than the white adipocytes, with a cytoplasm having multiple small lipid droplets, and high amount of mitochondria.

Beige adipocytes (also called brite adipocytes) are Ucp1+, multilocular adipocytes within white adipose tissue (WAT) that are capable of thermogenesis, the process of heat generation. Beige adipocytes are an inducible form of thermogenic adipocytes that sporadically reside within WAT depots. Similar to brown adipocytes, beige adipocytes possess abundant cristae-dense mitochondria that express UCP1 and multilocular lipid droplet. The presence of active BAT containing both classical brown and beige adipocytes is also found in the cervical, supraclavicular, mediastinal, paravertebral, and suprarenal regions.

A non-limiting, exemplary lineage of beige, white, and brown adipocytes from stem cells is shown in

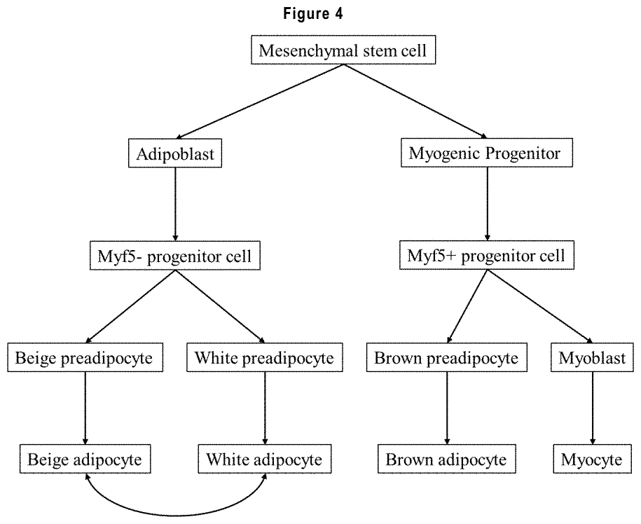

FIG. 4. Beige, white and brown adipocytes originate from the mesoderm. The mesenchymal stem cells can be committed to either an adipogenic lineage of Myf5-negative cells (Myf5− progenitor cells) or a myogenic lineage of Myf5-positive cells (Myf5+ progenitor cells). Myf5 is a key myogenic regulatory factor. The Myf5+ progenitor cells differentiate into the committed brown preadipocytes and myoblasts, which differentiate into brown adipocytes and myocytes, respectively. The Myf5− progenitor cells can be induced to differentiate into the committed beige preadipocytes and white preadipocytes, which differentiate into beige adipocytes and white adipocytes, respectively. White adipocytes can transdifferentiate into beige adipocytes.

The present technology relates to diseases or disorders characterized by abnormal numbers, ratios or bodily distributions of beige adipocytes, beige preadipocytes, white adipocytes, white preadipocytes, or immediate progenitors thereof with respect to each other.

Genes Signatures

Cell state transitions (i.e., a transition in a cell's state from a first cell state to a second cell state, e.g., differentiation) are characterized by a change in expression of genes in the cell. Changes in gene expression may be quantified as, e.g., an increase in mRNA expressed for a specific gene or a decrease in mRNA expressed for another specific gene; especially significant here may be mRNAs that encode transcription factors. Collectively, the sum of multiple differences in gene expression between one cell type or cells of one lineage relative to another cell type or cells of another lineage are referred to herein as a gene signature.

Any one of a number of methods and metrics may be used to identify gene signatures. Non-limiting examples include single cell and bulk RNA sequencing with or without prior cell sorting (e.g., fluorescence activated cell sorting (FACS) and flow cytometry). When developing a gene signature, it may be useful to first characterize the cell type or cells of a specific lineage by proteins that are characteristic of the cell type or cells of a specific lineage. Illustrative proteins include the thermoregulatory markers like UCP1, CIDEA, or Cox8b, and other markers such as Epst11, Prdm16, Cox7a, PGC1a, Tbx1, Tmem26 and Tnsfrsf9. Ussar et al., Sci Transl Med. 6(247): 247ra103 (2014).

Knowing the gene signature for each cell type or cells of a specific lineage provides insight into what genes impact or are associated with the process of transition to other cell types and/or differentiation of progenitor cells.

Gene signatures can be used to identify particular cells as being on-lineage, and other cells as being “progenitor” cells or intermediate cells along a transition trajectory towards the on-lineage cell type.

Genes that are differentially expressed and positively associated with the promotion of beige adipocyte lineage progression and/or differentiation to beige preadipocyte are listed in Table 1.

| | | Gene | Gene | Network |
|---|---|---|---|---|
| | Gene | EntrezID | Directionality | Module |
| 0 | JUN | 3725 | down | 0 |
| 1 | ID2 | 3398 | down | 0 |
| 2 | ZFP36 | 55552 | down | 0 |
| 3 | BAMBI | 25805 | down | 0 |
| 4 | HERPUD1 | 9709 | down | 0 |
| 5 | NCK1 | 4690 | down | 0 |
| 6 | MYC | 4609 | down | 0 |
| 7 | SQSTM1 | 8878 | down | 0 |
| 8 | NFKBIA | 4792 | down | 0 |
| 9 | IER3 | 8870 | down | 0 |
| 10 | TIPARP | 25976 | down | 0 |
| 11 | HES1 | 3280 | down | 0 |
| 12 | MRPL12 | 6182 | up | 1 |
| 13 | MIF | 268 | up | 1 |
| 14 | BIRC5 | 332 | up | 1 |
| 15 | CDK1 | 983 | up | 1 |
| 16 | UBE2C | 11065 | up | 1 |
| 17 | CYB561 | 1534 | down | 1 |
| 18 | HSD17B11 | 51170 | down | 1 |
| 19 | CISD1 | 55847 | up | 2 |
| 20 | TOMM70A | 9868 | up | 2 |
| 21 | RRS1 | 23212 | up | 2 |
| 22 | TRAP1 | 7190 | up | 2 |
| 23 | FYN | 2534 | down | 2 |
| 24 | PLSCR1 | 5359 | down | 2 |
| 25 | DLD | 1738 | up | 3 |
| 26 | GAPDH | 2597 | up | 3 |
| 27 | CDK4 | 1019 | up | 3 |
| 28 | MRPS16 | 51021 | up | 3 |
| 29 | GALE | 2582 | up | 3 |
| 30 | CIRBP | 1153 | down | 3 |
| 31 | CAT | 10249 | up | 4 |
| 32 | FOS | 2353 | down | 4 |
| 33 | NUCB2 | 4925 | down | 4 |
| 34 | S100A13 | 6284 | down | 4 |
| 35 | RSU1 | 6251 | down | 4 |
| 36 | ASAH1 | 427 | down | 4 |
| 37 | PFKL | 5211 | up | 5 |
| 38 | TIMP2 | 7077 | down | 5 |
| 39 | COL1A1 | 1277 | down | 5 |
| 40 | MYL9 | 10398 | down | 5 |
| 41 | LOXL1 | 4016 | down | 5 |
| 42 | COL4A1 | 1282 | down | 5 |
| 43 | CEBPA | 1050 | up | 6 |
| 44 | SCP2 | 10388 | up | 6 |
| 45 | LPGAT1 | 9926 | up | 6 |
| 46 | NT5DC2 | 64943 | up | 6 |
| 47 | GNB5 | 10681 | up | 6 |
| 48 | DNM1 | 1759 | down | 6 |
| 49 | LAP3 | 51056 | up | 7 |
| 50 | HSPA4 | 3308 | up | 7 |
| 51 | MMP2 | 4313 | down | 7 |
| 52 | PTGS2 | 5743 | down | 7 |
| 53 | SSBP2 | 23635 | down | 7 |
| 54 | RTN2 | 6253 | down | 7 |
| 55 | PPARG | 5468 | up | 8 |
| 56 | VAT1 | 6570 | down | 8 |
| 57 | GAA | 2548 | down | 8 |
| 58 | PROS1 | 5627 | down | 8 |
| 59 | B4GAT1 | 11041 | down | 8 |
| 60 | HSPD1 | 3329 | up | 9 |
| 61 | NOLC1 | 9221 | up | 9 |
| 62 | SDHB | 6390 | up | 9 |
| 63 | PNP | 5539 | down | 9 |
| 64 | DRAP1 | 10589 | down | 9 |
| 65 | PGAM1 | 5223 | up | 10 |
| 66 | PRSS23 | 11098 | down | 10 |
| 67 | IGFBP3 | 3486 | down | 10 |
| 68 | TPM1 | 7168 | down | 10 |
| 69 | ILK | 3611 | down | 10 |
| 70 | TIMM9 | 26520 | up | 11 |
| 71 | CCNB2 | 9133 | up | 11 |
| 72 | SCRN1 | 9805 | down | 11 |
| 73 | FHL2 | 2274 | down | 11 |
| 74 | KDM5B | 10765 | down | 11 |
| 75 | IFRD2 | 7866 | up | 12 |
| 76 | GADD45B | 4616 | down | 12 |
| 77 | EGFR | 1956 | down | 12 |
| 78 | GRN | 2896 | down | 12 |
| 79 | SERPINE1 | 5054 | down | 12 |
| 80 | MPC2 | 25874 | up | 13 |
| 81 | STMN1 | 3925 | up | 13 |
| 82 | PARP1 | 142 | up | 13 |
| 83 | UBE2A | 7319 | up | 13 |
| 84 | GSTZ1 | 2954 | up | 14 |
| 85 | TMEM50A | 23585 | down | 14 |
| 86 | CLTB | 1212 | down | 14 |
| 87 | GNAI2 | 2771 | down | 14 |
| 88 | SCARB1 | 949 | up | 15 |
| 89 | FKBP14 | 55033 | down | 15 |
| 90 | CALU | 813 | down | 15 |
| 91 | STXBP1 | 6812 | down | 15 |
| 92 | HTRA1 | 5654 | down | 16 |
| 93 | UGDH | 7358 | down | 16 |
| 94 | PLA2G4A | 5321 | down | 16 |
| 95 | EXT1 | 2131 | down | 16 |
| 96 | HADH | 3030 | up | 17 |
| 97 | PEX11A | 8800 | up | 17 |
| 98 | CTSL | 1514 | down | 17 |
| 99 | NENF | 29937 | down | 17 |
| 100 | ETFB | 2109 | up | 18 |
| 101 | HSD17B10 | 3028 | up | 18 |
| 102 | PXMP2 | 5827 | up | 18 |
| 103 | CIAPIN1 | 57019 | up | 19 |
| 104 | TGFBR2 | 7048 | down | 19 |
| 105 | EGR1 | 1958 | down | 19 |
| 106 | DNAJC15 | 29103 | up | 20 |
| 107 | P4HA2 | 8974 | down | 20 |
| 108 | GNAS | 2778 | down | 20 |
| 109 | FKBP4 | 2288 | up | 21 |
| 110 | APP | 351 | down | 21 |
| 111 | HSPA1A | 3303 | down | 21 |
| 112 | SMARCA4 | 6597 | up | 22 |
| 113 | ITGB5 | 3693 | down | 22 |
| 114 | EPB41L2 | 2037 | down | 22 |
| 115 | BZW2 | 28969 | up | 23 |
| 116 | GSTM2 | 2946 | down | 23 |
| 117 | ICAM1 | 3383 | down | 23 |
| 118 | VDAC1 | 7416 | up | 24 |
| 119 | ISOC1 | 51015 | up | 24 |
| 120 | CYCS | 54205 | up | 25 |
| 121 | G3BP1 | 10146 | up | 25 |
| 122 | CD320 | 51293 | up | 26 |
| 123 | MYLK | 4638 | up | 26 |
| 124 | EIF4EBP1 | 1978 | up | 27 |
| 125 | PHGDH | 26227 | up | 27 |
| 126 | CEBPD | 1052 | down | 28 |
| 127 | CHIC2 | 26511 | down | 28 |

In Table 1 and associated embodiments:
“Gene ID”: at the time of filing the present disclosure, the World Wide Web at ncbi.nlm.nih.gov/gene provides a description of and the nucleic acid sequence for each GeneID listed in Table 1; the contents of each of which is incorporated herein by reference in its entirety.
“Up” indicates a gene for which an increase in expression and/or activity in the progenitor cell is associated with the gene signature.
“Down” indicates a gene for which an decrease in expression and/or activity in the progenitor cell is associated with the gene signature.
A “network module” (sometimes also referred to as “module”) is a set of genes whose activity and/or expression are mutually predictive and, individually and collectively, are correlated with regard to a cell state change, which correlation may be positive or negative. That is, a module may contain genes that are positively associated with the cell state transition—such that an increase in expression and/or activity of the gene associated with the cell state transition; as well as genes that are negatively associated with the cell state transition such that a decrease in expression and/or activity of the gene associated with the cell state transition.

In certain embodiments, a network module includes genes in addition (or substituted for) to those exemplified in Table 1, which should be viewed as illustrative and not limiting unless expressly provided, namely with genes with correlated expression. A correlation, e.g., by the method of Pearson or Spearman, is calculated between a query gene expression profile for the desired cell state transition and one or more of the exemplary genes recited in the module. Those genes with a correlation with one or more genes of the module of at significance level below p=0.05 (e.g., 0.04, 0.03, 0.02, 0.01, 0.005, 0.001, 0.0005, 0.0001, or less) can be added to, or substituted for, other genes in the module.

“Activation of a network module” refers to a perturbation that modulates expression and/or activity of 2 or more genes (e.g., 3, 4, 5, 6 . . . genes; or about 10, 20, 25, 30, 40, 50, 60, 70, 75, 80, 85, 90, 95, or 100%) within a module, which modulation may be an increase or decrease in expression and/or activity of the gene as consonant with the modules described in Table 1. In certain embodiments, a perturbation activates multiple network modules for the desired cell state transition, such as 2, 3, 4, 5, 6, 7, 8, 9, 10, 11, 12, 13, 14, 15, 16, 17, 18, 19, 20, 21, 22, 23, 24, 25, 26, 27, 28 or more modules.

In some embodiments, one or more genes of network module 0 are modulated. In some embodiments, the presents relate to the activation of network module 0, e.g., one or more of (inclusive of all of) JUN, ID2, ZFP36, BAMBI, HERPUD1, NCK1, MYC, SQSTM1, NFKBIA, IER3, TIPARP, and HES1. In some embodiments, the modulation is upmodulation or downmodulation as described in Gene Directionality column of Table 1.

In some embodiments, one or more genes of network module 1 are modulated. In some embodiments, the presents relate to the activation of network module 1, e.g., one or more of (inclusive of all of) MRPL12, MIF, BIRC5, CDK1, UBE2C, CYB561, and HSD17B11. In some embodiments, the modulation is upmodulation or downmodulation as described in Gene Directionality column of Table 1.

In some embodiments, one or more genes of network module 2 are modulated. In some embodiments, the presents relate to the activation of network module 2, e.g., one or more of (inclusive of all of) CISD1, TOMM70A, RRS1, TRAP1, FYN, and PLSCR1. In some embodiments, the modulation is upmodulation or downmodulation as described in Gene Directionality column of Table 1.

In some embodiments, one or more genes of network module 3 are modulated. In some embodiments, the presents relate to the activation of network module 3, e.g., one or more of (inclusive of all of) DLD, GAPDH, CDK4, MRPS16, GALE, and CIRBP. In some embodiments, the modulation is upmodulation or downmodulation as described in Gene Directionality column of Table 1.

In some embodiments, one or more genes of network module 4 are modulated. In some embodiments, the presents relate to the activation of network module 4, e.g., one or more of (inclusive of all of) CAT, FOS, NUCB2, S100A13, RSU1, and ASAH1. In some embodiments, the modulation is upmodulation or downmodulation as described in Gene Directionality column of Table 1.

In some embodiments, one or more genes of network module 5 are modulated. In some embodiments, the presents relate to the activation of network module 5, e.g., one or more of (inclusive of all of) PFKL, TIMP2, COL1A1, MYL9, LOXL1, and COL4A1. In some embodiments, the modulation is upmodulation or downmodulation as described in Gene Directionality column of Table 1.

In some embodiments, one or more genes of network module 6 are modulated. In some embodiments, the presents relate to the activation of network module 6, e.g., one or more of (inclusive of all of) CEBPA, SCP2, LPGAT1, NT5DC2, GNB5 and DNM1. In some embodiments, the modulation is upmodulation or downmodulation as described in Gene Directionality column of Table 1.

In some embodiments, one or more genes of network module 7 are modulated. In some embodiments, the presents relate to the activation of network module 7, e.g., one or more of (inclusive of all of) LAP3, HSPA4, MMP2, PTGS2, SSBP2, and RTN2. In some embodiments, the modulation is upmodulation or downmodulation as described in Gene Directionality column of Table 1.

In some embodiments, one or more genes of network module 8 are modulated. In some embodiments, the presents relate to the activation of network module 8, e.g., one or more of (inclusive of all of) PPARG, VAT1, GAA, PROS1, and B4GAT1. In some embodiments, the modulation is upmodulation or downmodulation as described in Gene Directionality column of Table 1.

In some embodiments, one or more genes of network module 9 are modulated. In some embodiments, the presents relate to the activation of network module 9, e.g., one or more of (inclusive of all of) HSPD1, NOLC1, SDHB, PNP, and DRAP1. In some embodiments, the modulation is upmodulation or downmodulation as described in Gene Directionality column of Table 1.

In some embodiments, one or more genes of network module 10 are modulated. In some embodiments, the presents relate to the activation of network module 10, e.g., one or more of (inclusive of all of) PGAM1, PRSS23, IGFBP3, TPM1, and ILK. In some embodiments, the modulation is upmodulation or downmodulation as described in Gene Directionality column of Table 1.

In some embodiments, one or more genes of network module 11 are modulated. In some embodiments, the presents relate to the activation of network module 11, e.g., one or more of (inclusive of all of) TIMM9, CCNB2, SCRN1, FHL2, and KDM5B. In some embodiments, the modulation is upmodulation or downmodulation as described in Gene Directionality column of Table 1.

In some embodiments, one or more genes of network module 12 are modulated. In some embodiments, the presents relate to the activation of network module 12, e.g., one or more of (inclusive of all of) IFRD2, GADD45B, EGFR, GRN, and SERPINE1. In some embodiments, the modulation is upmodulation or downmodulation as described in Gene Directionality column of Table 1.

In some embodiments, one or more genes of network module 13 are modulated. In some embodiments, the presents relate to the activation of network module 13, e.g., one or more of (inclusive of all of) MPC2, STMN1, PARP1, and UBE2A. In some embodiments, the modulation is upmodulation or downmodulation as described in Gene Directionality column of Table 1.

In some embodiments, one or more genes of network module 14 are modulated. In some embodiments, the presents relate to the activation of network module 14, e.g., one or more of (inclusive of all of) GSTZ1, TMEM50A, CLTB, and GNAI2. In some embodiments, the modulation is upmodulation or downmodulation as described in Gene Directionality column of Table 1.

In some embodiments, one or more genes of network module 15 are modulated. In some embodiments, the presents relate to the activation of network module 15, e.g., one or more of (inclusive of all of) SCARB1, FKBP14, CALU, and STXBP1. In some embodiments, the modulation is upmodulation or downmodulation as described in Gene Directionality column of Table 1.

In some embodiments, one or more genes of network module 16 are modulated. In some embodiments, the presents relate to the activation of network module 16, e.g., one or more of (inclusive of all of) HTRA1, UGDH, PLA2G4A, and EXT1. In some embodiments, the modulation is upmodulation or downmodulation as described in Gene Directionality column of Table 1.

In some embodiments, one or more genes of network module 17 are modulated. In some embodiments, the presents relate to the activation of network module 17, e.g., one or more of (inclusive of all of) HADH, PEX11A, CTSL, and NENF. In some embodiments, the modulation is upmodulation or downmodulation as described in Gene Directionality column of Table 1.

In some embodiments, one or more genes of network module 18 are modulated. In some embodiments, the presents relate to the activation of network module 18, e.g., one or more of (inclusive of all of) ETFB, HSD17B10, and PXMP2. In some embodiments, the modulation is upmodulation or downmodulation as described in Gene Directionality column of Table 1.

In some embodiments, one or more genes of network module 19 are modulated. In some embodiments, the presents relate to the activation of network module 19, e.g., one or more of (inclusive of all of) CIAPIN1, TGFBR2, and EGR1. In some embodiments, the modulation is upmodulation or downmodulation as described in Gene Directionality column of Table 1.

In some embodiments, one or more genes of network module 20 are modulated. In some embodiments, the presents relate to the activation of network module 20, e.g., one or more of (inclusive of all of) DNAJC15, P4HA2, and GNAS. In some embodiments, the modulation is upmodulation or downmodulation as described in Gene Directionality column of Table 1.

In some embodiments, one or more genes of network module 21 are modulated. In some embodiments, the presents relate to the activation of network module 21, e.g., one or more of (inclusive of all of) FKBP4, APP, and HSPA1A. In some embodiments, the modulation is upmodulation or downmodulation as described in Gene Directionality column of Table 1.

In some embodiments, one or more genes of network module 22 are modulated. In some embodiments, the presents relate to the activation of network module 22, e.g., one or more of (inclusive of all of) SMARCA4, ITGB5, and EPB41L2. In some embodiments, the modulation is upmodulation or downmodulation as described in Gene Directionality column of Table 1.

In some embodiments, one or more genes of network module 23 are modulated. In some embodiments, the presents relate to the activation of network module 23, e.g., one or more of (inclusive of all of) BZW2, GSTM2, and ICAM1. In some embodiments, the modulation is upmodulation or downmodulation as described in Gene Directionality column of Table 1.

In some embodiments, one or more genes of network module 24 are modulated. In some embodiments, the presents relate to the activation of network module 24, e.g., one or both of VDAC1 and ISOC1. In some embodiments, the modulation is upmodulation or downmodulation as described in Gene Directionality column of Table 1.

In some embodiments, one or more genes of network module 25 are modulated. In some embodiments, the presents relate to the activation of network module 25, e.g., one or both of CYCS and G3BP1. In some embodiments, the modulation is upmodulation or downmodulation as described in Gene Directionality column of Table 1.

In some embodiments, one or more genes of network module 26 are modulated. In some embodiments, the presents relate to the activation of network module 26, e.g., one or both of CD320 and MYLK. In some embodiments, the modulation is upmodulation or downmodulation as described in Gene Directionality column of Table 1.

In some embodiments, one or more genes of network module 27 are modulated. In some embodiments, the presents relate to the activation of network module 27, e.g., one or both of EIF4EBP1 and PHGDH. In some embodiments, the modulation is upmodulation or downmodulation as described in Gene Directionality column of Table 1.

In some embodiments, one or more genes of network module 28 are modulated. In some embodiments, the presents relate to the activation of network module 28, e.g., one or both of CEBPD and CHIC2. In some embodiments, the modulation is upmodulation or downmodulation as described in Gene Directionality column of Table 1.

In some embodiments, the present methods alter a gene signature in the sample of cells, comprising an activation of a network module designated in the network module column of Table 1.

In some embodiments, the activation of the network module designated in the network module column of Table 1 comprises modulating expression and/or activity of 2 or more genes within a network module.

In some embodiments, the activation of the network module designated in the network module column of Table 1 comprises modulating expression and/or activity of all of the genes within a network module.

In some embodiments, the activation of the network module designated in the network module column of Table 1 comprises modulating expression and/or activity of 2 or more genes within 2 or more network modules. In some embodiments, the activation of the network module designated in the network module column of Table 1 comprises modulating expression and/or activity of 2 or more genes (e.g. 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, or 50 or more, or 51 or more, or 52 or more, or 53 or more, or 54 or more, or 55 or more, or 56 or more, or 57 or more, or 58 or more, or 59 or more, or 10 or more, or 61 or more, or 62 or more, or 63 or more, or 64 or more, or 65 or more, or 66 or more, or 67 or more, or 68 or more, or 69 or more, or 70 or more, or 71 or more, or 72 or more, or 73 or more, or 74 or more, or 75 or more, or 76 or more, or 77 or more, or 78 or more, or 79 or more, or 80 or more, or 81 or more, or 82 or more, or 83 or more, or 84 or more, or 85 or more, or 86 or more, or 87 or more, or 88 or more, or 89 or more, or 90 or more, or 91 or more, or 92 or more, or 93 or more, or 94 or more, or 95 or more, or 96 or more, or 97 or more, or 98 or more, or 99 or more, or 100 or more, or 101 or more, or 102 or more, or 103 or more, or 104 or more, or 105 or more, or 106 or more, or 107 or more, or 108 or more, or 109 or more, or 110 or more, or 111 or more, or 112 or more, or 113 or more, or 114 or more, or 115 or more, or 116 or more, or 117 or more, or 118 or more, or 119 or more, or 120 or more, or 121 or more, or 122 or more, or 123 or more, or 124 or more, or 125 or more, or 126 or more genes) within 2 or more network modules (e.g. 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more network modules).

At the time of filing the present disclosure, the World Wide Web at ncbi.nlm.nih.gov/gene provides a description of and the nucleic acid sequence for each Gene designated as an “up” gene in the gene directionality column of Table 1; the contents of each of which is incorporated herein by reference in its entirety. At the time of filing the present disclosure, the World Wide Web at ncbi.nlm.nih.gov/gene provides a description of and the nucleic acid sequence for each Gene listed in the genes designated as an “down” gene in the gene directionality column of Table 1; the contents of each of which is incorporated herein by reference in its entirety.

Perturbagens

A perturbagen useful in the present technology can be a small molecule, a biologic, a protein, a nucleic acid, such as a cDNA over-expressing a wild-type gene or an mRNA encoding a wild-type gene, or any combination of any of the foregoing. Illustrative perturbagens useful in the present technology and capable of promoting beige adipocyte lineage differentiation are listed in Table 3.

TABLE 3
A list of perturbagens.

| Perturbagen | | Molecular Weight | | |
|---|---|---|---|---|
| No. | Molecular Formula | (g/mol) | Dose | |
| 1 | C₂₇H₃₂N₈O₂ | 500.6 | 10 | μM |
| 2 | C₁₈H₁₄N₄O₂ | 318.3 | 10 | μM |
| 3 | C₁₇H₁₉N₃O | 281.35 | 10 | μM |
| 4 | C₁₇H₁₁Br₂NO₂S₂ | 485.2 | 40 | μM |
| 5 | C₂₆H₂₈N₄O₂ | 428.5 | 20 | μM |
| 6 | C₂₈H₂₇ClF₅NO | 524.0 | 0.37 | μM |
| 7 | C₂₂H₂₈N₄O₆ | 444.5 | 1.11 | μM |
| 8 | C₂₀H₁₉ClN₄O₃ | 398.8 | 0.04 | μM |
| 9 | C₂₇H₂₉NO₁₁ | 543.5 | 1 | nM |
| 10 | C₂₇H₂₉NO₁₀ | 527.5 | 10 | μM |
| 11 | C₁₉H₁₇ClFN₃O₅S | 453.9 | 10 | μM |
| 12 | C₁₄H₂₃N₃O₂ | 265.35 | 10 | μM |
| 13 | C₂₁H₁₉ClFNO₄S | 435.9 | | |

In various embodiments herein, a perturbagen of Table 3 encompasses the perturbagens named in Table 3. Thus, the named perturbagens of Table 3 represent examples of perturbagens of the present technology.

In Table 3, the effective in vitro concentration is the concentration of a perturbagen that is capable of increasing gene expression in a progenitor cell, as assayed, at least, by single cell gene expression profiling (GEP). Although the concentrations were determined in an in vitro assay, the concentrations may be relevant to a determination of in vivo dosages and such dosages may be used in clinic or in clinical testing.

In embodiments, a perturbagen used in the present technology is a variant of a perturbagen of Table 3. A variant may be a derivative, analog, enantiomer or a mixture of enantiomers thereof or a pharmaceutically acceptable salt, solvate, hydrate, co-crystal, clathrate, or polymorph of the perturbagen of Table 3. A variant of a perturbagen of Table 3 retains the biological activity of the perturbagen of Table 3.

Methods and Perturbagens for Directing a Change in Cell State

The present technology relates to the restoring the abnormal numbers, ratios or bodily distributions of beige adipocytes, beige preadipocytes, white adipocytes, white preadipocytes, or immediate progenitors thereof with respect to each other. Towards that, particular cellular changes in cell state can be matched to differential gene expression (which collectively define a gene signature), caused by exposure of a cell to a perturbagen. In embodiments, a change in cell state may be from one progenitor cell type to another progenitor cell type. For example, an adipocyte stem cell or a mesenchymal stem cell may change to an adipoblast; an adipoblast may change to an Myf5− progenitor; an Myf5− progenitor may change to a beige preadipocyte; and/or a white preadipocyte; a beige preadipocyte may change to a beige adipocyte; a white preadipocyte may change to a white adipocyte; and/or a white adipocyte may change to a beige adipocyte. In embodiments, a change in cell state may be from an upstream progenitor cell (e.g. an adipocyte stem cell, or a mesenchymal stem cell) to a downstream progenitor cell (an Myf5− progenitor cell, a white preadipocyte, or a beige preadipocyte). In embodiments, a change in cell state may be from the final non-differentiated cell into a differentiated cell (e.g. a white preadipocyte to a white adipocyte, or a beige preadipocyte to a beige adipocyte). Lastly, in embodiments, a change in cell state may be from one differentiated cell into a differentiated cell (e.g. transdifferentiation of a white adipocyte to a beige adipocyte).

An aspect of the present technology is a method for directing a change in cell state of a progenitor cell. The method includes a step of contacting a population of cells comprising a progenitor cell with at least one perturbagen selected from Table 3, or a variant thereof. In this aspect, the at least one perturbagen is capable of altering a gene signature in the progenitor cell. In one embodiment the progenitor cell is an adipocyte stem cell or a mesenchymal stem cell. In one embodiment, progenitor cell is a cell selected from the group of mesenchymal stem cell, adipoblast, Myf5− progenitor cell, white preadipocyte, white preadipocyte, and beige preadipocyte.

Another aspect of the present technology is a method for directing a change in cell state of a progenitor cell. This method includes a step of contacting a population of cells comprising a progenitor cell with at least one perturbagen capable of altering a gene signature in the progenitor cell. In this aspect, altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1. In one embodiment, the progenitor cell is an adipocyte stem cell or a mesenchymal stem cell. In one embodiment, progenitor cell is a cell selected from the group of mesenchymal stem cell, adipoblast, Myf5− progenitor cell, white preadipocyte, white preadipocyte, and beige preadipocyte.

Yet another aspect of the present technology is a method for directing a change in cell state of a progenitor cell. The method includes a step of contacting a population of cells comprising a progenitor cell with at least one perturbagen selected from Table 3, or a variant thereof, and capable of altering a gene signature in the progenitor cell. In this aspect, altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1. In one embodiment, the progenitor cell is an adipocyte stem cell or a mesenchymal stem cell. In one embodiment, progenitor cell is a cell selected from the group of mesenchymal stem cell, adipoblast, Myf5− progenitor cell, white preadipocyte, white preadipocyte, and beige preadipocyte.

In some embodiments, altering the gene signature comprises an increase in expression and/or activity in the progenitor cell of an activation of a network module designated in the network module column of Table 1. In some embodiments, the activation of one or more genes of the network module designated in the network module column of Table 1 comprises modulating expression and/or activity of 2 or more genes within a network module. In some embodiments, the activation of one or more genes of the network module designated in the network module column of Table 1 comprises modulating expression and/or activity of 3 or more genes within a network module. In some embodiments, the activation of one or more genes of the network module designated in the network module column of Table 1 comprises modulating expression and/or activity of 5 or more genes within a network module. In some embodiments, the activation of one or more genes of the network module designated in the network module column of Table 1 comprises modulating expression and/or activity of all genes within a network module. In some embodiments, altering the gene signature comprises an increase in expression and/or activity in the progenitor cell of two or more genes designated as an “up” gene in the gene directionality column of Table 1. In some embodiments, altering the gene signature comprises a decrease in expression and/or activity in the progenitor cell of one or more genes designated as a “down” gene in the gene directionality column of Table 1. In some embodiments, altering the gene signature comprises a decrease in expression and/or activity in the progenitor cell of two or more genes designated as a “down” gene in the gene directionality column of Table 1.

In some embodiments, the progenitor cell is an adipocyte stem cell or a mesenchymal stem cell. In some embodiments, the step of contacting a population of cells comprising a progenitor cell with a perturbagen causes a change in the cell state. Such change in cell state can provide an increase in the number of one or more of mesenchymal stem cells, adipoblasts, Myf5− progenitor cells, beige preadipocytes, and beige adipocytes. In some embodiments, the beige adipocytes can be derived from the canonical developmental pathway, example of which shown in FIG. 4. In some embodiments, the beige adipocytes can be derived from a developmental pathway that does not include the canonical developmental pathway disclosed herein.

In embodiments, the change in cell state (e.g. by contacting a population of cells comprising a progenitor cell with a perturbagen) provides an increase in the number of beige adipocytes. Without being bound by theory, this change may brought about by a number of mechanisms, such as (1) increasing differentiation of adipocyte stem cells or mesenchymal stem cells towards every lineage described in FIG. 4, including differentiation to Myf5− progenitor cells and Myf5− progenitor cells and their downstream cells; (2) selective differentiation of adipocyte stem cells or mesenchymal stem cells towards Myf5− progenitor cells, and their downstream cells; (3) increased and/or selective differentiation of adipocyte stem cells or mesenchymal stem cells towards Myf5− progenitor cells their downstream cells, including white preadipocytes, white adipocytes, beige preadipocytes and beige adipocytes; (4) increased and/or selective differentiation of Myf5− progenitor cells to beige preadipocytes; (5) increased differentiation of beige preadipocytes to beige adipocytes; (6) increased transdifferentiation of white adipocytes to beige adipocytes; (7) increased or decreased survival of cells disclosed herein (for example, in FIG. 4); and a combination of any two or more thereof.

Accordingly, in some embodiments, additionally, or alternatively, the change in cell state provides an increase in the number of one or more of adipoblasts, Myf5− progenitor cells, beige preadipocytes, and/or beige adipocytes. In some embodiments, the increase in the number of adipoblasts, Myf5− progenitor cells, beige preadipocytes, and/or beige adipocytes is relative to the number of adipoblasts, Myf5− progenitor cells, beige preadipocytes, and/or beige adipocytes obtained from a population of progenitor cells that are not contacted with the at least one perturbagen. In some embodiments, the increase in the number of adipoblasts, Myf5− progenitor cells, beige preadipocytes, and/or beige adipocytes is relative to the number of adipoblasts, Myf5− progenitor cells, beige preadipocytes, and/or beige adipocytes obtained from a population of progenitor cells prior to contacting with the at least one perturbagen.

Additionally, or alternatively, in some embodiments, the change in cell state provides an increase in the number of beige adipocytes. In some embodiments, the increase in the number of beige adipocytes is relative to the number of beige adipocytes obtained from a population of progenitor cells that are not contacted with the at least one perturbagen. In some embodiments, the increase in the number of beige adipocytes is relative to the number of beige adipocytes obtained from a population of progenitor cells prior to contacting with the at least one perturbagen.

Additionally or alternatively, in some embodiments, the change in cell state provides the lack of a substantial increase in the number of cells of white preadipocytes and/or white adipocytes. In some embodiments, the lack of substantial increase in the number of preadipocytes and/or white adipocytes is relative to the number of preadipocytes and/or white adipocytes obtained from a population of progenitor cells that are not contacted with the at least one perturbagen. In some embodiments, the lack of substantial increase in the number of preadipocytes and/or white adipocytes is relative to the number of preadipocytes and/or white adipocytes obtained from a population of progenitor cells prior to contacting with the at least one perturbagen.

Additionally, or alternatively, in some embodiments, the change in cell state provides a substantial decrease in the number of cells of white preadipocytes and/or white adipocytes. In some embodiments, the substantial decrease in the number of preadipocytes and/or white adipocytes is relative to the number of preadipocytes and/or white adipocytes obtained from a population of progenitor cells that are not contacted with the at least one perturbagen. In some embodiments, the substantial decrease in the number of preadipocytes and/or white adipocytes is relative to the number of preadipocytes and/or white adipocytes obtained from a population of progenitor cells prior to contacting with the at least one perturbagen.

In some embodiments, the change in cell state provides a substantial increase in the number of other committed cells of myogenic lineage selected from the group of Myf5+ progenitor cells, myoblasts, brown preadipocytes, brown adipocytes and skeletal muscle cells. In some embodiments, the change in cell state does not provide a substantial increase in the number of other committed cells of myogenic lineage selected from the group of Myf5+ progenitor cells, myoblasts, brown preadipocytes, brown adipocytes and skeletal muscle cells. In some embodiments, the substantial increase in the number of other committed cells of myogenic lineage selected from the group of Myf5+ progenitor cells, myoblasts, brown preadipocytes, brown adipocytes and skeletal muscle cells is relative to the number of other committed cells of myogenic lineage selected from the group of Myf5+ progenitor cells, myoblasts, brown preadipocytes, brown adipocytes and skeletal muscle cells obtained from a population of progenitor cells that are not contacted with the at least one perturbagen. In some embodiments, the substantial increase in the number of other committed cells of myogenic lineage selected from the group of Myf5+ progenitor cells, myoblasts, brown preadipocytes, brown adipocytes and skeletal muscle cells is relative to the number of other committed cells of myogenic lineage selected from the group of Myf5+ progenitor cells, myoblasts, brown preadipocytes, brown adipocytes and skeletal muscle cells obtained from a population of progenitor cells prior to contacting with the at least one perturbagen.

In some embodiments, the change in cell state provides a substantial decrease in the number of other committed cells of myogenic lineage selected from the group of Myf5+ progenitor cells, myoblasts, brown preadipocytes, brown adipocytes and skeletal muscle cells. In some embodiments, the substantial decrease in the number of other committed cells of myogenic lineage selected from the group of Myf5+ progenitor cells, myoblasts, brown preadipocytes, brown adipocytes and skeletal muscle cells is relative to the number of other committed cells of myogenic lineage selected from the group of Myf5+ progenitor cells, myoblasts, brown preadipocytes, brown adipocytes and skeletal muscle cells obtained from a population of progenitor cells that are not contacted with the at least one perturbagen. In some embodiments, the substantial decrease in the number of other committed cells of myogenic lineage selected from the group of Myf5+ progenitor cells, myoblasts, brown preadipocytes, brown adipocytes and skeletal muscle cells is relative to the number of other committed cells of myogenic lineage selected from the group of Myf5+ progenitor cells, myoblasts, brown preadipocytes, brown adipocytes and skeletal muscle cells obtained from a population of progenitor cells prior to contacting with the at least one perturbagen.

Additionally, or alternatively, in some embodiments, the increase in the number of adipoblasts, Myf5− progenitor cells, beige preadipocytes, and/or beige adipocytes is due in part to decreased cell proliferation of other committed cells of myogenic lineage selected from the group of Myf5+ progenitor cells, myoblasts, brown preadipocytes, brown adipocytes and skeletal muscle cells. Additionally, or alternatively, in some embodiments, the increase in the number of adipoblasts, Myf5− progenitor cells, beige preadipocytes, and/or beige adipocytes is due in part to increased survival of other committed cells of myogenic lineage selected from the group of Myf5+ progenitor cells, myoblasts, brown preadipocytes, brown adipocytes and skeletal muscle cells. Additionally, or alternatively, in some embodiments, the increase in the number of adipoblasts, Myf5− progenitor cells, beige preadipocytes, and/or beige adipocytes is due in part to an increased lifespan of the adipoblasts, Myf5− progenitor cells, beige preadipocytes, and/or beige adipocytes. Additionally, or alternatively, in some embodiments, the increase in the number of adipoblasts, Myf5− progenitor cells beige preadipocytes, and/or beige adipocytes is due in part to a decreased cell death of the adipoblasts, Myf5− progenitor cells, beige preadipocytes, and/or beige adipocytes.

Additionally, or alternatively, in some embodiments, the increase in the number of adipoblasts, Myf5− progenitor cells, beige preadipocytes, and/or beige adipocytes is due in part to decreased cell proliferation of white preadipocytes and /or white adipocytes. Additionally, or alternatively, in some embodiments, the increase in the number of adipoblasts, Myf5− progenitor cells, beige preadipocytes, and/or beige adipocytes is due in part to decreased survival of white preadipocytes and /or white adipocytes. Additionally, or alternatively, in some embodiments, the increase in the number of adipoblasts, Myf5− progenitor cells, beige preadipocytes, and/or beige adipocytes is due in part to transdifferentiation of white adipocytes to beige adipocytes.

Additionally, or alternatively, in some embodiments, the increase in the number of beige adipocytes is due in part to an increased lifespan of the beige adipocytes. Additionally, or alternatively, in some embodiments, the increase in the number of beige adipocytes is due in part to a decreased cell death of the beige adipocytes. Additionally, or alternatively, in some embodiments, the increase in the number of beige adipocytes is due in part to a decreased apoptosis of the beige adipocytes.

In some embodiments, the increase in the number of beige adipocytes is due in part to decreased lifespan of white preadipocytes and/or white adipocytes. In some embodiments, the increase in the number of beige adipocytes is due in part to increased cell death of white preadipocytes and/or white adipocytes. In some embodiments, the increase in the number of beige adipocytes is due in part to increased apoptosis of white preadipocytes and/or white adipocytes. In some embodiments, the increase in the number of beige adipocytes is due in part to increased transdifferentiation of white adipocytes to beige preadipocytes.

In some embodiments, the increase in the number of beige adipocytes is due in part to increased lifespan of beige preadipocytes. In some embodiments, the increase in the number of beige adipocytes is due in part to decreased cell death of beige preadipocytes. In some embodiments, the increase in the number of beige adipocytes is due in part to decreased apoptosis of beige preadipocytes. In some embodiments, the increase in the number of beige adipocytes is due in part to increased differentiation of beige preadipocytes to beige adipocytes.

In some embodiments, the increase in the number of beige adipocytes is due in part to increased lifespan of Myf5− progenitor cells. In some embodiments, the increase in the number of beige adipocytes is due in part to decreased cell death of Myf5− progenitor cells. In some embodiments, the increase in the number of beige adipocytes is due in part to decreased apoptosis of Myf5− progenitor cells. In some embodiments, the increase in the number of beige adipocytes is due in part to increased differentiation of Myf5− progenitor cells. In some embodiments, the increase in the number of beige adipocytes is due in part to increased differentiation of Myf5− progenitor cells to beige preadipocytes.

In some embodiments, the increase in the number of beige adipocytes is due in part to increased lifespan of adipoblasts. In some embodiments, the increase in the number of beige adipocytes is due in part to decreased cell death of adipoblast. In some embodiments, the increase in the number of beige adipocytes is due in part to decreased apoptosis of adipoblast. In some embodiments, the increase in the number of beige adipocytes is due in part to increased differentiation of adipoblast. In some embodiments, the increase in the number of beige adipocytes is due in part to increased differentiation of adipoblasts to Myf5− progenitor cells.

In some embodiments, the increase in the number of beige adipocytes is due in part to increased lifespan of mesenchymal stem cells. In some embodiments, the increase in the number of beige adipocytes is due in part to decreased cell death of mesenchymal stem cells. In some embodiments, the increase in the number of beige adipocytes is due in part to decreased apoptosis of mesenchymal stem cells. In some embodiments, the increase in the number of beige adipocytes is due in part to increased differentiation of mesenchymal stem cells. In some embodiments, the increase in the number of beige adipocytes is due in part to increased differentiation of mesenchymal stem cells to adipoblasts.

In some embodiments, the increase in the number of beige adipocytes is due in part to decreased cell death of other committed cells of myogenic lineage selected from the group of Myf5+ progenitor cells, myoblasts, brown preadipocytes, brown adipocytes and skeletal muscle cells. In some embodiments, the increase in the number of beige adipocytes is due in part to decreased apoptosis of other committed cells of myogenic lineage selected from the group of Myf5+ progenitor cells, myoblasts, brown preadipocytes, brown adipocytes and skeletal muscle cells. In some embodiments, the increase in the number of beige adipocytes is due in part to increased cell death of other committed cells of myogenic lineage selected from the group of Myf5+ progenitor cells, myoblasts, brown preadipocytes, brown adipocytes and skeletal muscle cells. In some embodiments, the increase in the number of beige adipocytes is due in part to increased apoptosis of other committed cells of myogenic lineage selected from the group of Myf5+ progenitor cells, myoblasts, brown preadipocytes, brown adipocytes and skeletal muscle cells.

Methods for determining the extension of the lifespan of a specific cell type or a reduction of cell death is well known in the art. As examples, markers for dying cells, e.g., caspases can be detected, or dyes for dead cells, e.g., methylene blue, may be used.

In some embodiments, the increase in the number of beige adipocytes is due in part to a change of cell state from mesenchymal stem cells into committed cells of myogenic lineage selected from the group of Myf5+ progenitor cells, myoblasts, brown preadipocytes, brown adipocytes and skeletal muscle cells;

and/or adipoblasts, Myf5− progenitor cells, beige preadipocytes, and/or beige adipocytes. In some embodiments, the increase in the number of beige adipocytes is due in part to a change of cell state from mesenchymal stem cells into adipoblasts, Myf5− progenitor cells, beige preadipocytes, and/or beige adipocytes.

In some embodiments, the number of mesenchymal stem cells is decreased. In some embodiments, the decrease in the number of mesenchymal stem cells is due in part to decreased cell proliferation of the mesenchymal stem cells. In some embodiments, the decrease in the number of mesenchymal stem cells is due in part to a decreased lifespan of the mesenchymal stem cells. In some embodiments, the decrease in the number of mesenchymal stem cells is due in part to an increased cell death of the mesenchymal stem cells. In some embodiments, the decrease in the number of mesenchymal stem cells is due in part to an increased apoptosis of the mesenchymal stem cells. In some embodiments, the decrease in the number of mesenchymal stem cells is relative to the number of mesenchymal stem cells in a population of progenitor cells that are not contacted with the at least one perturbagen. In some embodiments, the decrease in the number of mesenchymal stem cells is relative to the number of mesenchymal stem cells in a population of cells comprising one or more of mesenchymal stem cells, adipoblasts, Myf5− progenitor cells, and beige preadipocytes, wherein the population of cells is not contacted with the at least one perturbagen. In some embodiments, the decrease in the number of mesenchymal stem cells is relative to the number of mesenchymal stem cells in a population of progenitor cells prior to contacting with the at least one perturbagen. In some embodiments, the decrease in the number of mesenchymal stem cells is due in part to an increased differentiation of the mesenchymal stem cells. In some embodiments, the decrease in the number of mesenchymal stem cells is due in part to an increased differentiation of the mesenchymal stem cells to one or more of adipoblasts, Myf5− progenitor cells, and beige preadipocytes. In some embodiments, the decrease in the number of mesenchymal stem cells is due to a change of cell state from a progenitor cell into the committed cells of myogenic lineage selected from the group of Myf5+ progenitor cells, myoblasts, brown preadipocytes, brown adipocytes and skeletal muscle cells; and/or adipoblasts, Myf5− progenitor cells, beige preadipocytes, and/or beige adipocytes.

In other embodiments, the number of mesenchymal stem cells is increased. In some embodiments, the increase in the number of mesenchymal stem cells is due in part to increased cell proliferation of the mesenchymal stem cells. In some embodiments, the increase in the number of mesenchymal stem cells is due in part to an increased lifespan of the mesenchymal stem cells. In some embodiments, the increase in the number of mesenchymal stem cells is due in part to decreased cell death among the mesenchymal stem cells. In some embodiments, the increase in the number of mesenchymal stem cells is relative to the number of mesenchymal stem cells in a population of progenitor cells that are not contacted with the at least one perturbagen. In some embodiments, the increase in the number of mesenchymal stem cells is relative to the number of mesenchymal stem cells in a population of progenitor cells prior to contacting with the at least one perturbagen.

Additionally, or alternatively, in some embodiments, the number of beige adipocytes is increased after contacting the population of progenitor cells comprising an adipocyte stem cell or mesenchymal stem cell with the at least one perturbagen. In some embodiments, the number of beige preadipocytes, and/or beige adipocytes is increased after contacting the population of progenitor cells comprising an adipocyte stem cell or mesenchymal stem cell with the at least one perturbagen. In some embodiments, the number of beige preadipocytes, and/or beige adipocytes is increased after contacting the population of progenitor cells comprising an adipocyte stem cell or mesenchymal stem cell with the at least one perturbagen. In some embodiments, the number of Myf5− progenitor cells, beige preadipocytes, and/or beige adipocytes is increased after contacting the population of progenitor cells comprising an adipocyte stem cell or mesenchymal stem cell with the at least one perturbagen. In some embodiments, the number of adipoblasts, Myf5− progenitor cells, beige preadipocytes, and/or beige adipocytes is increased after contacting the population of progenitor cells comprising an adipocyte stem cell or mesenchymal stem cell with the at least one perturbagen. In some embodiments, the number of committed cells of myogenic lineage selected from the group of Myf5+ progenitor cells, myoblasts, brown preadipocytes, brown adipocytes and/or skeletal muscle cells is increased after contacting the population of cells comprising a adipocyte stem cell or mesenchymal stem cell with the at least one perturbagen. In some embodiments, the number of white preadipocytes and/or white adipocytes is decreased after contacting the population of progenitor cells comprising an adipocyte stem cell or mesenchymal stem cell with the at least one perturbagen. In some embodiments, the number of committed cells of myogenic lineage selected from the group of Myf5+ progenitor cells, myoblasts, brown preadipocytes, brown adipocytes and/or skeletal muscle cells is decreased after contacting the population of cells comprising a adipocyte stem cell or mesenchymal stem cell with the at least one perturbagen.

In some embodiments, the ratio of the number of beige adipocytes to the number of white adipocytes is increased relative to the ratio in the population of progenitor cells prior to contacting with the at least one perturbagen. In some embodiments, the ratio of the number of beige adipocytes to the number of white preadipocytes is increased relative to the ratio in the population of progenitor cells prior to contacting with the at least one perturbagen. In some embodiments, the ratio of the number of beige adipocytes to the number of Myf5− progenitors is increased relative to the ratio in the population of progenitor cells that are not contacted with the at least one perturbagen. In some embodiments, the ratio of the number of beige adipocytes to the number of adipoblasts is increased relative to the ratio in the population of progenitor cells that are not contacted with the at least one perturbagen. In some embodiments, the ratio of the number of the beige adipocytes to the number of the committed cells of myogenic lineage selected from the group of Myf5+ progenitor cells, myoblasts, brown preadipocytes, brown adipocytes and/or skeletal muscle cells is increased relative to the ratio in the population of progenitor cells that are not contacted with the at least one perturbagen. In some embodiments, the ratio of the number of beige adipocytes to the number of cells of myogenic lineage is increased relative to the ratio in the population of progenitor cells prior to contacting with the at least one perturbagen, wherein the cells of myogenic lineage are selected from the group of Myf5+ progenitor cells, myoblasts, brown preadipocytes, brown adipocytes and/or skeletal muscle cells.

In some embodiments, the ratio of the number of white adipocytes to the number of white preadipocytes is decreased relative to the ratio in the population of progenitor cells prior to contacting with the at least one perturbagen. In some embodiments, the ratio of the number of white adipocytes to the number of Myf5− progenitors is decreased relative to the ratio in the population of progenitor cells prior to contacting with the at least one perturbagen. In some embodiments, the ratio of the number of white adipocytes to the number of adipoblasts is decreased relative to the ratio in the population of progenitor cells prior to contacting with the at least one perturbagen. In some embodiments, the ratio of the number of white adipocytes to the number of mesenchymal stem cells is decreased relative to the ratio in the population of progenitor cells prior to contacting with the at least one perturbagen. In some embodiments, the ratio of the number of white adipocytes to the number of cells of myogenic lineage is decreased relative to the ratio in the population of progenitor cells prior to contacting with the at least one perturbagen, wherein the cells of myogenic lineage are selected from the group of Myf5+ progenitor cells, myoblasts, brown preadipocytes, brown adipocytes and/or skeletal muscle cells.

In some embodiments, the ratio of the number of beige preadipocytes to the number of Myf5− progenitors is increased relative to the ratio in the population of progenitor cells prior to contacting with the at least one perturbagen. In some embodiments, the ratio of the number of beige preadipocytes to the number of adipoblasts is increased relative to the ratio in the population of progenitor cells prior to contacting with the at least one perturbagen. In some embodiments, the ratio of the number of beige preadipocytes to the number of mesenchymal stem cells is increased relative to the ratio in the population of progenitor cells prior to contacting with the at least one perturbagen. In some embodiments, the ratio of the number of beige preadipocytes to the number of cells of myogenic lineage is increased relative to the ratio in the population of progenitor cells prior to contacting with the at least one perturbagen, wherein the cells of myogenic lineage are selected from the group of Myf5+ progenitor cells, myoblasts, brown preadipocytes, brown adipocytes and/or skeletal muscle cells.

In some embodiments, the ratio of the number of Myf5− progenitors to the number of adipoblasts is increased relative to the ratio in the population of progenitor cells prior to contacting with the at least one perturbagen. In some embodiments, the ratio of the number of Myf5− progenitors to the number of mesenchymal stem cells is increased relative to the ratio in the population of progenitor cells prior to contacting with the at least one perturbagen. In some embodiments, the ratio of the number of Myf5− progenitors to the number of cells of myogenic lineage is increased relative to the ratio in the population of progenitor cells prior to contacting with the at least one perturbagen, wherein the cells of myogenic lineage are selected from the group of Myf5+ progenitor cells, myoblasts, brown preadipocytes, brown adipocytes and/or skeletal muscle cells. In some embodiments, the ratio of the number of adipoblasts to the number of mesenchymal stem cells is increased relative to the ratio in the population of progenitor cells prior to contacting with the at least one perturbagen. In some embodiments, the ratio of the number of adipoblasts the number of cells of myogenic lineage is increased relative to the ratio in the population of progenitor cells prior to contacting with the at least one perturbagen, wherein the cells of myogenic lineage are selected from the group of Myf5+ progenitor cells, myoblasts, brown preadipocytes, brown adipocytes and/or skeletal muscle cells.

In some embodiments, the ratio of the number of beige adipocytes to the number of white adipocytes is increased relative to the ratio in the population of progenitor cells that is not contacted with the at least one perturbagen. In some embodiments, the ratio of the number of beige adipocytes to the number of white preadipocytes is increased relative to the ratio in the population of progenitor cells that is not contacted with the at least one perturbagen. In some embodiments, the ratio of the number of beige adipocytes to the number of Myf5− progenitors is increased relative to the ratio in the population of progenitor cells that are not contacted with the at least one perturbagen. In some embodiments, the ratio of the number of beige adipocytes to the number of adipoblasts is increased relative to the ratio in the population of progenitor cells that are not contacted with the at least one perturbagen. In some embodiments, the ratio of the number of beige adipocytes to the number of the committed cells of myogenic lineage selected from the group of Myf5+ progenitor cells, myoblasts, brown preadipocytes, brown adipocytes and/or skeletal muscle cells is increased relative to the ratio in the population of progenitor cells that are not contacted with the at least one perturbagen. In some embodiments, the ratio of the number of beige adipocytes to the number of cells of myogenic lineage is increased relative to the ratio in the population of progenitor cells that is not contacted with the at least one perturbagen, wherein the cells of myogenic lineage are selected from the group of Myf5+ progenitor cells, myoblasts, brown preadipocytes, brown adipocytes and/or skeletal muscle cells.

In some embodiments, the ratio of the number of white adipocytes to the number of white preadipocytes is decreased relative to the ratio in the population of progenitor cells that is not contacted with the at least one perturbagen. In some embodiments, the ratio of the number of white adipocytes to the number of Myf5− progenitors is decreased relative to the ratio in the population of progenitor cells that is not contacted with the at least one perturbagen. In some embodiments, the ratio of the number of white adipocytes to the number of adipoblasts is decreased relative to the ratio in the population of progenitor cells that is not contacted with the at least one perturbagen. In some embodiments, the ratio of the number of white adipocytes to the number of mesenchymal stem cells is decreased relative to the ratio in the population of progenitor cells that is not contacted with the at least one perturbagen. In some embodiments, the ratio of the number of white adipocytes to the number of cells of myogenic lineage is decreased relative to the ratio in the population of progenitor cells that is not contacted with the at least one perturbagen, wherein the cells of myogenic lineage are selected from the group of Myf5+ progenitor cells, myoblasts, brown preadipocytes, brown adipocytes and/or skeletal muscle cells.

In some embodiments, the ratio of the number of beige preadipocytes to the number of Myf5− progenitors is increased relative to the ratio in the population of progenitor cells that is not contacted with the at least one perturbagen. In some embodiments, the ratio of the number of beige preadipocytes to the number of adipoblasts is increased relative to the ratio in the population of progenitor cells that is not contacted with the at least one perturbagen. In some embodiments, the ratio of the number of beige preadipocytes to the number of mesenchymal stem cells is increased relative to the ratio in the population of progenitor cells that is not contacted with the at least one perturbagen. In some embodiments, the ratio of the number of beige preadipocytes to the number of cells of myogenic lineage is increased relative to the ratio in the population of progenitor cells that is not contacted with the at least one perturbagen, wherein the cells of myogenic lineage are selected from the group of Myf5+ progenitor cells, myoblasts, brown preadipocytes, brown adipocytes and/or skeletal muscle cells.

In some embodiments, the ratio of the number of Myf5− progenitors to the number of adipoblasts is increased relative to the ratio in the population of progenitor cells that is not contacted with the at least one perturbagen. In some embodiments, the ratio of the number of Myf5− progenitors to the number of mesenchymal stem cells is increased relative to the ratio in the population of progenitor cells that is not contacted with the at least one perturbagen. In some embodiments, the ratio of the number of Myf5− progenitors to the number of cells of myogenic lineage is increased relative to the ratio in the population of progenitor cells that is not contacted with the at least one perturbagen, wherein the cells of myogenic lineage are selected from the group of Myf5+ progenitor cells, myoblasts, brown preadipocytes, brown adipocytes and/or skeletal muscle cells.

In some embodiments, the ratio of the number of adipoblasts to the number of mesenchymal stem cells is increased relative to the ratio in the population of progenitor cells that is not contacted with the at least one perturbagen. In some embodiments, the ratio of the number of adipoblasts to the number of cells of myogenic lineage is increased relative to the ratio in the population of progenitor cells that is not contacted with the at least one perturbagen, wherein the cells of myogenic lineage are selected from the group of Myf5+ progenitor cells, myoblasts, brown preadipocytes, brown adipocytes and/or skeletal muscle cells.

In some embodiments, the number of adipoblasts is increased relative to the number of adipoblasts in the population of progenitor cells prior to contacting with the at least one perturbagen. In some embodiments, the number of adipoblasts is increased relative to the number of adipoblasts in the population of progenitor cells that is not contacted with the at least one perturbagen. In some embodiments, the number of Myf5− progenitors is increased relative to the number of Myf5− progenitors in the population of progenitor cells prior to contacting with the at least one perturbagen. In some embodiments, the number of Myf5− progenitors is increased relative to the number of Myf5− progenitors in the population of progenitor cells that is not contacted with the at least one perturbagen. In some embodiments, the number of white preadipocytes is decreased relative to the number of white preadipocytes in the population of progenitor cells prior to contacting with the at least one perturbagen. In some embodiments, the number of white preadipocytes is decreased relative to the number of white preadipocytes in the population of progenitor cells that is not contacted with the at least one perturbagen.

In some embodiments, the number of white adipocytes is decreased relative to the number of white adipocytes in the population of progenitor cells prior to contacting with the at least one perturbagen. In some embodiments, the number of white adipocytes is decreased relative to the number of white adipocytes in the population of progenitor cells that is not contacted with the at least one perturbagen. In some embodiments, the number of beige preadipocytes is increased relative to the number of beige preadipocytes in the population of progenitor cells prior to contacting with the at least one perturbagen. In some embodiments, the number of beige preadipocytes is increased relative to the number of beige preadipocytes in the population of progenitor cells that is not contacted with the at least one perturbagen. In some embodiments, the number of beige adipocytes is increased relative to the number of beige adipocytes in the population of progenitor cells prior to contacting with the at least one perturbagen. In some embodiments, the number of beige adipocytes is increased relative to the number of beige adipocytes in the population of progenitor cells that is not contacted with the at least one perturbagen.

Methods for counting cells are well known in the art. Non-limiting examples include hemocytometry, flow cytometry, and cell sorting techniques, e.g., fluorescence activated cell sorting (FACS).

Additionally or alternatively, in some embodiments, the at least one perturbagen is selected from Table 3, or a variant thereof, comprises at least 2, at least 3, at least 4, or at least 5 perturbagens selected from Table 3, or variants thereof.

Additionally or alternatively, in some embodiments, the one or more genes are selected from the genes designated as an “up” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, or 55 genes selected from the genes designated as an “up” gene in the gene directionality column of Table 1. In some embodiments, the one or more genes designated as an “up” gene in the gene directionality column of Table 1 are selected from MRPL12, MIF, BIRC5, CDK1, UBE2C, CISD1, TOMM70A, RRS1, TRAP1, DLD, GAPDH, CDK4, MRPS16, GALE, CAT, PFKL, CEBPA, SCP2, LPGAT1, NT5DC2, GNB5, LAP3, HSPA4, PPARG, HSPD1, NOLC1, SDHB, PGAM1, TIMM9, CCNB2, IFRD2, MPC2, STMN1, PARP1, UBE2A, GSTZ1, SCARB1, HADH, PEX11A, ETFB, HSD17B10, PXMP2, CIAPIN1, DNAJC15, FKBP4, SMARCA4, BZW2, VDAC1, ISOC1, CYCS, G3BP1, CD320, MYLK, EIF4EBP1, and/or PHGDH.

Additionally or alternatively, in some embodiments, the one or more genes are selected from the genes designated as an “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, or 73 genes selected from the genes designated as an “down” gene in the gene directionality column of Table 1. In some embodiments, one or more genes are selected from JUN, ID2, ZFP36, BAMBI, HERPUD1, NCK1, MYC, SQSTM1, NFKBIA, IER3, TIPARP, HES1, CYB561, HSD17B11, FYN, PLSCR1, CIRBP, FOS, NUCB2, S100A13, RSU1, ASAH1, TIMP2, COL1A1, MYL9, LOXL1, COL4A1, DNM1, MMP2, PTGS2, SSBP2, RTN2, VAT1, GAA, PROS1, B4GAT1, PNP, DRAP1, PRSS23, IGFBP3, TPM1, ILK, SCRN1, FHL2, KDM5B, GADD45B, EGFR, GRN, SERPINE1, TMEM50A, CLTB, GNAI2, FKBP14, CALU, STXBP1, HTRA1, UGDH, PLA2G4A, EXT1, CTSL, NENF, TGFBR2, EGR1, P4HA2, GNAS, APP, HSPA1A, ITGB5, EPB41L2, GSTM2, ICAM1, CEBPD, and/or CHIC2. In embodiments, the at least one perturbagen selected from Table 3, or a variant thereof, comprises at least 2, at least 3, at least 4, or at least 5 perturbagens selected from Table 3, or variants thereof.

In embodiments, altering the gene signature comprises increased expression and/or increased activity in the progenitor cell of one or more genes selected from the genes designated as an “up” gene in the gene directionality column of Table 1. In embodiments, the one or more genes selected from the genes designated as an “up” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, or 55 genes. In some embodiments, the genes selected from an activation of a network module designated in the network module column of Table 1 comprise at least one of MRPL12, MIF, BIRC5, CDK1, UBE2C, CISD1, TOMM70A, RRS1, TRAP1, DLD, GAPDH, CDK4, MRPS16, GALE, CAT, PFKL, CEBPA, SCP2, LPGAT1, NT5DC2, GNB5, LAP3, HSPA4, PPARG, HSPD1, NOLC1, SDHB, PGAM1, TIMM9, CCNB2, IFRD2, MPC2, STMN1, PARP1, UBE2A, GSTZ1, SCARB1, HADH, PEX11A, ETFB, HSD17B10, PXMP2, CIAPIN1, DNAJC15, FKBP4, SMARCA4, BZW2, VDAC1, ISOC1, CYCS, G3BP1, CD320, MYLK, EIF4EBP1, and/or PHGDH.

In embodiments, altering the gene signature comprises decreased expression and/or decreased activity in the progenitor cell of one or more genes selected from the genes designated as an “down” gene in the gene directionality column of Table 1. In embodiments, the one or more genes selected from the genes designated as an “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, or 73genes selected from the genes designated as an “down” gene in the gene directionality column of Table 1. In embodiments, the one or more genes selected from the genes designated as an “down” gene in the gene directionality column of Table 1 comprise at least one of JUN, ID2, ZFP36, BAMBI, HERPUD1, NCK1, MYC, SQSTM1, NFKBIA, IER3, TIPARP, HES1, CYB561, HSD17B11, FYN, PLSCR1, CIRBP, FOS, NUCB2, S100A13, RSU1, ASAH1, TIMP2, COL1A1, MYL9, LOXL1, COL4A1, DNM1, MMP2, PTGS2, SSBP2, RTN2, VAT1, GAA, PROS1, B4GAT1, PNP, DRAP1, PRSS23, IGFBP3, TPM1, ILK, SCRN1, FHL2, KDM5B, GADD45B, EGFR, GRN, SERPINE1, TMEM50A, CLTB, GNAI2, FKBP14, CALU, STXBP1, HTRA1, UGDH, PLA2G4A, EXT1, CTSL, NENF, TGFBR2, EGR1, P4HA2, GNAS, APP, HSPA1A, ITGB5, EPB41L2, GSTM2, ICAM1, CEBPD, and CHIC2.

In embodiments, an increase in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more increase in gene expression relative to a cell that has not been contacted with a perturbagen and/or relative to a cell that has been contacted with a no treatment control (e.g. including an appropriate vehicle only control such as DMSO). Likewise, a decrease in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more decrease in gene expression relative to a cell that has not been contacted with a perturbagen and/or relative to a cell that has been contacted with a no treatment control (e.g. including an appropriate vehicle only control such as DMSO).

In various embodiments, an increase in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater increase in gene expression relative to a cell that has not been contacted with a perturbagen and/or relative to a cell that has been contacted with a no treatment control (e.g. including an appropriate vehicle only control such as DMSO). Likewise, a decrease in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater decrease in gene expression relative to a cell that has not been contacted with a perturbagen and/or relative to a cell that has been contacted with a no treatment control (e.g. including an appropriate vehicle only control such as DMSO).

Additionally or alternatively, in some embodiments, the contacting the population of progenitor cells occurs in vitro or ex vivo. In embodiments, contacting the population of cells comprising a progenitor cell occurs in vitro or ex vivo.

Additionally or alternatively, in some embodiments, the contacting the population of progenitor cells occurs in vivo in a subject. In some embodiments, the subject is a human. In some embodiments, the subject is an adult human. In some embodiments, the subject has an abnormal number of one or more of beige adipocytes, white adipocytes, beige preadipocytes, and/or white preadipocytes, or a disease or disorder characterized thereby. In some embodiments, the subject has a reduced number of beige adipocytes and/or beige preadipocytes, or a disease or disorder characterized thereby. In some embodiments, the subject has an increased number of white adipocytes, or a disease or disorder characterized thereby. In some embodiments, the subject has a reduced number of beige adipocytes, and an increased number of white adipocytes, or a disease or disorder characterized thereby. In some embodiments, the subject has an abnormal bodily distribution of one or more of beige adipocytes, white adipocytes, beige preadipocytes, and/or white preadipocytes, or a disease or disorder characterized thereby. Methods for evaluating abnormal numbers or bodily distribution of one or more of beige adipocytes, white adipocytes, beige preadipocytes, and/or white preadipocytes are well known in the art. Exemplary abnormal bodily distribution of one or more of beige adipocytes, white adipocytes, beige preadipocytes, and/or white preadipocytes includes, but is not limited to, e.g., (1) an increased number of white adipocytes in visceral WAT (such as located inside the peritoneum, and distributed around internal organs (e.g., stomach, liver, intestines, and kidneys)); (2) a decreased number of beige adipocytes and/or beige preadipocytes in visceral WAT (such as located inside the peritoneum, and distributed around internal organs (e.g., stomach, liver, intestines, and kidneys)); (3) an increased number of white adipocytes in subcutaneous WAT; (4) a decreased number of beige adipocytes and/or beige preadipocytes in subcutaneous WAT; or a combination of any two or more thereof. In some embodiments, the subject has an abnormal ratio of the number of beige adipocytes to the number of white adipocytes, the number of beige adipocytes to the number of beige preadipocytes, the number of beige adipocytes to the number of white preadipocytes, and/or the number of beige adipocytes to the number of brown adipocytes; or a disease or disorder characterized thereby. In some embodiments, the subject has one or more WAT (such as visceral WAT and/or subcutaneous WAT) having the subject has an abnormal ratio of the number of beige adipocytes to the number of white adipocytes, the number of beige adipocytes to the number of beige preadipocytes, the number of beige adipocytes to the number of white preadipocytes, and/or the number of beige adipocytes to the number of brown adipocytes; or a disease or disorder characterized thereby.

Additionally or alternatively, in some embodiments, the method provides an increase in expression and/or activity of uncoupling protein 1 (UCP1), optionally as compared to the expression and/or activity the absence of a perturbagen. In some embodiments, the method provides an increase in expression and/or activity of uncoupling protein 1 (UCP1), optionally as compared to the expression and/or activity prior to contacting with the at least one perturbagen. Additionally or alternatively, in some embodiments, the method increases the amount or extent of non-shivering thermogenesis, optionally as compared to the amount or extent of non-shivering thermogenesis to the absence of a perturbagen. In some embodiments, the method increases the amount or extent of non-shivering thermogenesis, optionally as compared to the amount or extent of non-shivering thermogenesis prior to contacting with the at least one perturbagen. Additionally or alternatively, in some embodiments, the method increases or stimulates the beiging and/or browning of white adipose tissue (WAT), optionally as compared to the beiging and/or browning of WAT in the absence of a perturbagen. In some embodiments, the method increases or stimulates the beiging and/or browning of white adipose tissue (WAT), optionally as compared to the beiging and/or browning of WAT prior to contacting with the at least one perturbagen. Additionally or alternatively, in some embodiments, the method enhance energy expenditure by reducing lipids stored within adipose tissue.

Additionally or alternatively, in some embodiments, the method increases the amount and/or activity of one or more of miR-193a/b, miR-365, miR-328, miR-378, miR-30b/c, miR-455, and miR-32, optionally as compared to expression and/or activity as compared to the absence of a perturbagen. In some embodiments, the method decreases the amount and/or activity of one or more of miR-27, miR-34a, miR-133, and miR-155, optionally as compared to the expression and/or activity in the absence of a perturbagen.

In an aspect, the present technology provides a method for promoting the formation of a beige adipocyte or an immediate progenitor thereof. The method includes a step of exposing a starting population of stem/progenitor cells comprising an adipocyte stem cell or a mesenchymal stem cell to a perturbation having a perturbation signature that promotes the transition of the starting population of stem/progenitor cells into adipoblasts, Myf5− progenitor cellsbeige preadipocytes, and/or beige adipocytes. In this aspect, the perturbation signature comprises increased expression and/or activity of one or more of genes selected from the genes designated as an “up” gene in the gene directionality column of Table 1 and/or a decreased expression and/or activity in the progenitor cell selected from an adipocyte stem cell or a mesenchymal stem cell of one or more genes selected from the genes designated as an “down” gene in the gene directionality column of Table 1. Embodiments associated with the above aspects are likewise relevant to the present aspect. In other words, each of the embodiments mentioned above for the above aspects may be revised/adapted to be applicable to the present aspect.

In another aspect, the present technology provides a method of increasing a quantity of a beige adipocyte cell or immediate progenitors thereof. The method includes a step of exposing a starting population of stem/progenitor cells comprising an adipocyte stem cell or a mesenchymal stem cell to a pharmaceutical composition that promotes the formation of lineage specific progenitor population selected from adipoblasts, Myf5− progenitor cells, beige preadipocytes. The pharmaceutical composition promotes the transition of a primitive stem/progenitor population into the lineage specific progenitor population that has the capacity to differentiate into beige adipocytes or immediate progenitors thereof. In this aspect, the pharmaceutical composition comprises at least one perturbagen selected from Table 3, or a variant thereof. Embodiments associated with the above aspects are likewise relevant to the present aspect. In other words, each of the embodiments mentioned above for the above aspects may be revised/adapted to be applicable to the present aspect.

In yet another aspect, the present technology provides a perturbagen for use in any herein disclosed method.

In a further aspect, the present technology provides a pharmaceutical composition comprising perturbagen for use in any herein disclosed method.

Methods and Perturbagens for Treating a Disease or Disorder

The present technology relates to the treatment of diseases or disorders abnormal numbers, ratios or bodily distributions of beige adipocytes, beige preadipocytes, white adipocytes, white preadipocytes, or immediate progenitors thereof with respect to each other. The ability of a perturbagen to specifically promote the formation of beige adipocytes would be valuable in designing a therapeutic composition for such diseases of disorders. As example, for a disease characterized by a reduced number of beige adipocytes, a therapeutic composition comprising a perturbagen that increases the number of beige adipocytes could be beneficial and/or a disease (including the same disease) that would benefit from increased number of beige adipocytes could be treated by a therapeutic composition comprising a perturbagen that increases the number of beige adipocytes. As example, for a disease characterized by an increased number of white adipocytes, a therapeutic composition comprising a perturbagen that transdifferentiates white adipocytes to beige adipocytes and/or promotes the formation of beige adipocytes could be beneficial and/or a disease (including the same disease) that would benefit from increased number of beige adipocytes and/or reduced number of white adipocytes could be treated by a therapeutic composition comprising a perturbagen that transdifferentiates white adipocytes to beige adipocytes and/or promotes the formation of beige adipocytes.

An aspect of the present technology is a method for promoting the formation of a beige adipocyte, or an immediate progenitor thereof. The method comprises exposing a starting population of stem/progenitor cells comprising a non-lineage committed mesenchymal stem cell nor adipocyte stem cell to a perturbation having a perturbation signature that promotes the transition of the starting population of stem/progenitor cells into a beige adipocyte, wherein the perturbation signature comprises increased expression and/or activity of one or more of genes selected from the genes designated as an “up” gene in the gene directionality column of Table 1 and/or a decreased expression and/or activity in the non-lineage committed adipocyte stem cell or mesenchymal stem cell of one or more genes selected from the genes designated as an “down” gene in the gene directionality column of Table 1. In some embodiments, the perturbation signature comprises an increase in expression and/or activity in the progenitor cell of an activation of a network module designated in the network module column of Table 1. In some embodiments, the activation of one or more genes of the network module designated in the network module column of Table 1 comprises modulating expression and/or activity of 2 or more genes within a network module. In some embodiments, the activation of one or more genes of the network module designated in the network module column of Table 1 comprises modulating expression and/or activity of 3 or more genes within a network module. In some embodiments, the activation of one or more genes of the network module designated in the network module column of Table 1 comprises modulating expression and/or activity of 5 or more genes within a network module. In some embodiments, the activation of one or more genes of the network module designated in the network module column of Table 1 comprises modulating expression and/or activity of all genes within a network module. In some embodiments, the perturbation signature comprises an increase in expression and/or activity in the progenitor cell of two or more genes designated as an “up” gene in the gene directionality column of Table 1. In some embodiments, the perturbation signature comprises a decrease in expression and/or activity in the progenitor cell of one or more genes designated as a “down” gene in the gene directionality column of Table 1. In some embodiments, the perturbation signature comprises a decrease in expression and/or activity in the progenitor cell of two or more genes designated as a “down” gene in the gene directionality column of Table 1.

An aspect of the present technology is a method of increasing a quantity of beige adipocytes, or immediate progenitors thereof in a subject in need thereof. The method comprises exposing a starting population of stem/progenitor cells comprising a non-lineage committed adipocyte stem cell or mesenchymal stem cell to a pharmaceutical composition that promotes the formation of lineage specific progenitor population selected from adipoblasts, Myf5− progenitor cells, beige preadipocytes, and/or beige adipocytes, the pharmaceutical composition promoting the transition of a primitive stem/progenitor population into the lineage specific progenitor population that has the capacity to differentiate into beige preadipocytes, and/or beige adipocytes or immediate progenitors thereof, wherein the pharmaceutical composition comprises at least one perturbagen selected from Table 3, or a variant thereof.

An aspect of the present technology is a method of decreasing a quantity of white adipocytes, or immediate progenitors thereof in a subject in need thereof. The method comprises exposing a starting population of stem/progenitor cells comprising a non-lineage committed adipocyte stem cell or mesenchymal stem cell to a pharmaceutical composition that promotes the formation of lineage specific progenitor population selected from adipoblasts, Myf5− progenitor cells, beige preadipocytes, and/or beige adipocytes, the pharmaceutical composition promoting the transition of a primitive stem/progenitor population into the lineage specific progenitor population that has the capacity to differentiate into beige preadipocytes, and/or beige adipocytes or immediate progenitors thereof, wherein the pharmaceutical composition comprises at least one perturbagen selected from Table 3, or a variant thereof.

An aspect of the present technology is a method for treating a disease or disorder characterized by an abnormal number of beige adipocytes, white adipocytes, beige preadipocytes, and/or white preadipocytes in a subject in need thereof. The method comprises (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell. In embodiments, the abnormal number of beige adipocytes, white adipocytes, beige preadipocytes, and/or white preadipocytes is a beige adipocyte and/or beige preadipocyte deficiency. In embodiments, the abnormal number of beige adipocytes, white adipocytes, beige preadipocytes, and/or white preadipocytes is an elevated level of white adipocytes.

An aspect of the present technology is a method for treating a disease or disorder characterized by an increased number of white adipocytes, and/or white preadipocytes in a subject in need thereof. The method comprises (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

An aspect of the present technology is a method for treating a disease or disorder characterized by a decreased number of beige adipocytes, and/or beige preadipocytes in a subject in need thereof. The method comprises (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

An aspect of the present technology is a method for treating a disease or disorder characterized by a decreased capacity to produce beige adipocytes, and/or beige preadipocytes in a subject in need thereof. The method comprises (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

An aspect of the present technology is a method for treating a disease or disorder characterized by an abnormal bodily distribution of beige adipocytes, white adipocytes, beige preadipocytes, and/or white preadipocytes in a subject in need thereof. The method comprises (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

An aspect of the present technology is a method for treating a disease or disorder characterized by decreased amount or extent of non-shivering thermogenesis in a subject in need thereof. The method comprises (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

An aspect of the present technology is a method for treating a disease or disorder characterized by decreased amount or extent of beiging and/or browning of white adipose tissue (WAT) in a subject in need thereof. The method comprises (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

An aspect of the present technology is a method for increasing the amount or extent of non-shivering thermogenesis in a subject in need thereof. The method comprises (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

An aspect of the present technology is a method for increasing the amount or extent of beiging and/or browning of white adipose tissue (WAT) in a subject in need thereof. The method comprises (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

An aspect of the present technology is a method for enhancing energy expenditure by reducing lipids stored within adipose tissue in a subject in need thereof. The method comprises (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

In some embodiments of any of the methods disclosed herein, the subject has a reduced number of beige adipocytes and/or beige preadipocytes, or a disease or disorder characterized thereby. In some embodiments of any of the methods disclosed herein, the subject has an increased number of white adipocytes, or a disease or disorder characterized thereby. In some embodiments of any of the methods disclosed herein, the subject has a reduced number of beige adipocytes, and an increased number of white adipocytes, or a disease or disorder characterized thereby. In some embodiments of any of the methods disclosed herein, the subject has an abnormal bodily distribution of one or more of beige adipocytes, white adipocytes, beige preadipocytes, and/or white preadipocytes, or a disease or disorder characterized thereby. Exemplary abnormal bodily distribution of one or more of beige adipocytes, white adipocytes, beige preadipocytes, and/or white preadipocytes includes, but is not limited to, e.g., (1) an increased number of white adipocytes in visceral WAT (such as located inside the peritoneum, and distributed around internal organs (e.g., stomach, liver, intestines, and kidneys)); (2) a decreased number of beige adipocytes and/or beige preadipocytes in visceral WAT (such as located inside the peritoneum, and distributed around internal organs (e.g., stomach, liver, intestines, and kidneys)); (3) an increased number of white adipocytes in subcutaneous WAT; (4) a decreased number of beige adipocytes and/or beige preadipocytes in subcutaneous WAT; or a combination of any two or more thereof. In some embodiments, the subject has an abnormal ratio of the number of beige adipocytes to the number of white adipocytes, the number of beige adipocytes to the number of beige preadipocytes, the number of beige adipocytes to the number of white preadipocytes, and/or the number of beige adipocytes to the number of brown adipocytes; or a disease or disorder characterized thereby. In some embodiments, the subject has one or more WAT (such as visceral WAT and/or subcutaneous WAT) having the subject has an abnormal ratio of the number of beige adipocytes to the number of white adipocytes, the number of beige adipocytes to the number of beige preadipocytes, the number of beige adipocytes to the number of white preadipocytes, and/or the number of beige adipocytes to the number of brown adipocytes; or a disease or disorder characterized thereby.

In some embodiments of any of the methods disclosed herein, the disease or disorder is obesity, morbid obesity, morbid obesity prior to surgery, obesity linked inflammation, obesity linked gallbladder disease, obesity induced sleep apnea, hyperlipidemia, dyslipidemia, hypercholesterolemia, atherogenic dyslipidemia, fatty liver disease (FLD), including nonalcoholic fatty liver disease (NAFLD), Non-Alcoholic Steatohepatitis (NASH), insulin resistance, glucose intolerance, pre-diabetes, increased fasting glucose, diabetes mellitus, including type 2 diabetes mellitus, inflammation, including vascular inflammation, hypertension, endothelial dysfunction, dyslipidemia (e.g. high triglycerides, low HDL cholesterol and/or high LDL cholesterol), Prader-Willi Syndrome, insulin resistance, glucose intolerance, pre-diabetes, increased fasting glucose, diabetes mellitus, type 2 diabetes mellitus, inflammation, vascular inflammation, hypertension, endothelial dysfunction, atherosclerosis, arteriosclerosis, coronary heart disease, peripheral artery disease, stroke, or microvascular disease, arterial remodelling, cardiovascular disease (CVD), including atherosclerotic cardiovascular disease, metabolic syndrome (insulin resistance, hypertension, hyperlipidemia), a metabolic disorder, excess body weight, or a combination of any two or more thereof. In some embodiments, the method alleviates one or more symptoms selected from the group of abdominal obesity, a body mass index (BMI) of 30 or higher, a blood triglyceride level of 150 or higher, a blood HDL level of less than 40 or higher in men, a blood HDL level of 50 or higher in women, a blood pressure of 130/85 or higher, a fasting blood sugar of 110 or higher, hyperlipidemia, dyslipidemia, hypercholesterolemia, atherogenic dyslipidemia insulin resistance, glucose intolerance, pre-diabetes, increased fasting glucose, diabetes mellitus, or a combination of any two or more thereof.

In embodiments, the administering is systemic or local. In embodiments, the administering is via intraosseous injection or intraosseous infusion. In other embodiments, the administering of the cell is via intravenous injection or intravenous infusion.

In some embodiments, the methods described herein are where at least one perturbagen is administered on the basis of previously determining the patient exhibits an abnormal number of beige adipocytes, or a disease or disorder characterized thereby disclosed herein. In some embodiments, the methods described herein are where at least one perturbagen is administered on the basis of previously determining the patient exhibits an abnormal number of white adipocytes, or a disease or disorder characterized thereby disclosed herein. In some embodiments, the present technology relates to a method of treating a disease or disorder characterized by beige adipocytes and includes administering an effective amount of a perturbagen selected from Table 3 or a variant thereof to a subject in need thereof. In some embodiments, the present technology relates to a method of treating a disease or disorder characterized by elevated levels of white adipocytes and includes administering an effective amount of a perturbagen selected from Table 3 or a variant thereof to a subject in need thereof. In some embodiments, the present technology relates to a method of treating a disease or disorder characterized by an abnormal number of beige adipocytes, e.g., a bleeding disorder and includes administering an effective amount of a perturbagen selected from Table 3 or a variant thereof to a subject in need thereof. In other embodiments, the present technology relates to a method of treating a disease or disorder is selected from obesity, morbid obesity, morbid obesity prior to surgery, obesity linked inflammation, obesity linked gallbladder disease, obesity induced sleep apnea, hyperlipidemia, dyslipidemia, hypercholesterolemia, atherogenic dyslipidemia, fatty liver disease (FLD), including nonalcoholic fatty liver disease (NAFLD), Non-Alcoholic Steatohepatitis (NASH), insulin resistance, glucose intolerance, pre-diabetes, increased fasting glucose, diabetes mellitus, including type 2 diabetes mellitus, inflammation, including vascular inflammation, hypertension, endothelial dysfunction, dyslipidemia(e.g. high triglycerides, low HDL cholesterol and/or high LDL cholesterol), Prader-Willi Syndrome, insulin resistance, glucose intolerance, pre-diabetes, increased fasting glucose, diabetes mellitus, type 2 diabetes mellitus, inflammation, vascular inflammation, hypertension, endothelial dysfunction, atherosclerosis, arteriosclerosis, coronary heart disease, peripheral artery disease, stroke, or microvascular disease, arterial remodelling, cardiovascular disease (CVD), including atherosclerotic cardiovascular disease, metabolic syndrome (insulin resistance, hypertension, hyperlipidemia), a metabolic disorder, excess body weight, and a combination of any two or more thereof, and includes administering an effective amount of a perturbagen selected from Table 3 or a variant thereof to a subject in need thereof.

Another aspect of the present technology is a method for treating obesity. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating morbid obesity. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating morbid obesity prior to surgery. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating obesity linked inflammation. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating obesity linked gallbladder disease. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating obesity induced sleep apnea. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating hyperlipidemia. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating dyslipidemia. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating hypercholesterolemia. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating atherogenic dyslipidemia. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating fatty liver disease (FLD), including nonalcoholic fatty liver disease (NAFLD), and Non-Alcoholic Steatohepatitis (NASH). The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating insulin resistance. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating glucose intolerance. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating pre-diabetes. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating diabetes mellitus, including type 1 diabetes mellitus and type 2 diabetes mellitus. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating inflammation, including vascular inflammation. The method comprising a step of: (a) administering to a patient in need thereof a therapeutically effective amount of at least one perturbagen selected from Table 3, or a variant thereof;

or (b) administering to a patient in need thereof a cell, the cell having been contacted with at least one perturbagen selected from Table 3, or a variant thereof.

Another aspect of the present technology is a method for treating hypertension. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating endothelial dysfunction. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating endothelial dyslipidemia (e.g. high triglycerides, low HDL cholesterol and/or high LDL cholesterol). The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating Prader-Willi Syndrome. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating atherosclerosis. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating coronary heart disease. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating peripheral artery disease. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating stroke. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating microvascular disease. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating arterial remodelling. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating cardiovascular disease (CVD), including atherosclerotic cardiovascular disease. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating metabolic syndrome (insulin resistance, hypertension, hyperlipidemia). The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating a metabolic disorder. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

Another aspect of the present technology is a method for treating excess body weight. The method comprising a step of: (a) administering to a patient in need thereof a therapeutically effective amount of at least one perturbagen selected from Table 3, or a variant thereof; or (b) administering to a patient in need thereof a cell, the cell having been contacted with at least one perturbagen selected from Table 3, or a variant thereof.

In one aspect, the present technology relates to a method for treating a disease or disorder characterized by an abnormal ratio of the number of beige adipocytes to the number of one or more of adipoblasts, Myf5− progenitor cells, white preadipocytes, white adipocytes, beige preadipocytes, and/or cells of myogenic lineage selected from the group of Myf5+ progenitor cells, myoblasts, brown preadipocytes, brown adipocytes and skeletal muscle cells. This method includes (a) administering to a patient in need thereof at least one perturbagen, or a variant thereof, wherein the at least one perturbagen is capable of changing a gene signature in a progenitor cell or (b) administering to a patient in need thereof a cell, the cell having been contacted with at least one perturbagen, or a variant thereof, wherein the at least one perturbagen is capable of changing a gene signature in a progenitor cell.

In one aspect, the present technology relates to a method for treating a disease or disorder characterized by an abnormal ratio of the number of beige adipocytes to the number of white adipocytes. This method includes (a) administering to a patient in need thereof at least one perturbagen, or a variant thereof, wherein the at least one perturbagen is capable of changing a gene signature in a progenitor cell or (b) administering to a patient in need thereof a cell, the cell having been contacted with at least one perturbagen, or a variant thereof, wherein the at least one perturbagen is capable of changing a gene signature in a progenitor cell.

In some embodiments, the abnormal ratio comprises a decreased number of one or more of adipoblasts, Myf5− progenitor cells, beige preadipocytes. In some embodiments, the abnormal ratio comprises a decreased number of beige preadipocytes and/or beige preadipocytes. In some embodiments, the abnormal ratio comprises an increased number of white preadipocytes, and/or white adipocytes.

In embodiments, the administering occurs about once per day for one or more days. In embodiments, the administering occurs more than once per day for one or more days. In embodiments, the administering occurs at most once per day for one or more days. In embodiments, the administering occurs substantially continuously per administration period.

An aspect of the present technology is a method for selecting a subject for method of any of the methods disclosed herein. The method for selecting a subject comprises obtaining from a subject having the disease or disorder a sample of cells comprising a non-lineage committed adipocyte stem cell or mesenchymal stem cell; and contacting the sample of cells with least one perturbagen selected from Table 3 or a variant thereof, wherein the at least one perturbagen alters a gene signature in the sample of cells, the subject is selected as a patient.

An aspect of the present technology is a method for selecting a subject for method of any of the methods disclosed herein. The method for selecting a subject comprises obtaining from a subject having the disease or disorder a sample of cells comprising a non-lineage committed adipocyte stem cell or mesenchymal stem cell; and contacting the sample of cells with at least one perturbagen capable of altering a gene signature in a non-lineage committed adipocyte stem cell or mesenchymal stem cell, wherein the at least one perturbagen increases in the sample of cells the expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1.

An aspect of the present technology is a method for selecting a subject for method of any of the methods disclosed herein. The method for selecting a subject comprises obtaining from a subject having the disease or disorder a sample of cells comprising a non-lineage committed adipocyte stem cell or mesenchymal stem cell; and contacting the sample of cells with least one perturbagen selected from Table 3 or a variant thereof, wherein the at least one perturbagen increases in the sample of cells the expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1.

In some embodiments, the perturbation signature comprises an increase in expression and/or activity in the progenitor cell of an activation of a network module designated in the network module column of Table 1. In some embodiments, the activation of one or more genes of the network module designated in the network module column of Table 1 comprises modulating expression and/or activity of 2 or more genes within a network module. In some embodiments, the activation of one or more genes of the network module designated in the network module column of Table 1 comprises modulating expression and/or activity of 3 or more genes within a network module. In some embodiments, the activation of one or more genes of the network module designated in the network module column of Table 1 comprises modulating expression and/or activity of 5 or more genes within a network module. In some embodiments, the activation of one or more genes of the network module designated in the network module column of Table 1 comprises modulating expression and/or activity of all genes within a network module. In some embodiments, the perturbation signature comprises an increase in expression and/or activity in the progenitor cell of two or more genes designated as an “up” gene in the gene directionality column of Table 1. In some embodiments, the perturbation signature comprises an increase in expression and/or activity in the progenitor cell of one or more genes designated as an “up” gene in the gene directionality column of Table 1. In some embodiments, the one or more genes designated as an “up” gene in the gene directionality column of Table 1 are selected from MRPL12, MIF, BIRC5, CDK1, UBE2C, CISD1, TOMM70A, RRS1, TRAP1, DLD, GAPDH, CDK4, MRPS16, GALE, CAT, PFKL, CEBPA, SCP2, LPGAT1, NT5DC2, GNB5, LAP3, HSPA4, PPARG, HSPD1, NOLC1, SDHB, PGAM1, TIMM9, CCNB2, IFRD2, MPC2, STMN1, PARP1, UBE2A, GSTZ1, SCARB1, HADH, PEX11A, ETFB, HSD17B10, PXMP2, CIAPIN1, DNAJC15, FKBP4, SMARCA4, BZW2, VDAC1, ISOC1, CYCS, G3BP1, CD320, MYLK, EIF4EBP1, and/or PHGDH. In some embodiments, the perturbation signature comprises a decrease in expression and/or activity in the progenitor cell of one or more genes designated as a “down” gene in the gene directionality column of Table 1. In some embodiments, the perturbation signature comprises a decrease in expression and/or activity in the progenitor cell of one or more genes designated as a “down” gene in the gene directionality column of Table 1. In some embodiments, one or more genes designated as a “down” gene in the gene directionality column of Table 1 are selected from JUN, ID2, ZFP36, BAMBI, HERPUD1, NCK1, MYC, SQSTM1, NFKBIA, IER3, TIPARP, HES1, CYB561, HSD17B11, FYN, PLSCR1, CIRBP, FOS, NUCB2, S100A13, RSU1, ASAH1, TIMP2, COL1A1, MYL9, LOXL1, C0L4A1, DNM1, MMP2, PTGS2, SSBP2, RTN2, VAT1, GAA, PROS1, B4GAT1, PNP, DRAP1, PRSS23, IGFBP3, TPM1, ILK, SCRN1, FHL2, KDM5B, GADD45B, EGFR, GRN, SERPINE1, TMEM50A, CLTB, GNAI2, FKBP14, CALU, STXBP1, HTRA1, UGDH, PLA2G4A, EXT1, CTSL, NENF, TGFBR2, EGR1, P4HA2, GNAS, APP, HSPA1A, ITGB5, EPB41L2, GSTM2, ICAM1, CEBPD, and/or CHIC2. In some embodiments, the one or more genes are selected from the genes designated as a “down” gene, or “up” regulated gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, 100 or more, 101 or more, 102 or more, 103 or more, 104 or more, 105 or more, 106 or more, 107 or more, 108 or more, 109 or more, 110 or more, 111 or more, 112 or more, 113 or more, 114 or more, 115 or more, 116 or more, 117 or more, 118 or more, 119 or more, 120 or more, 121 or more, 122 or more, 123 or more, 124 or more, 125 or more, 126 or more, 127 or more, or 128 genes selected from the genes designated as a “down” gene, or “up” regulated gene in the gene directionality column of Table 1. In some embodiments, the at least one perturbagen is selected from Table 3, or a variant thereof, comprises at least 2, at least 3, at least 4, or at least 5 perturbagens selected from Table 3, or variants thereof. In some embodiments, the at least one perturbagen is selected from Table 3, or a variant thereof, comprises at least 2, at least 3, at least 4, or at least 5 perturbagens selected from Table 3, or variants thereof.

Diseases or Disorders

The term “disease or disorder” as used in context of the present technology refers to a disease or condition that is characterized by abnormal numbers, ratios or bodily distributions of beige adipocytes, beige preadipocytes, white adipocytes, white preadipocytes, or immediate progenitors thereof with respect to each other. The disease or disorder is impacted by the presence, level or activity of white adipose tissue, brown adipose tissue, plasma glucose concentration, plasma insulin level and/or body fat content. In some embodiments, the disease or disorder is a metabolic disorder or condition that includes, but is not limited to, Metabolic Syndrome, impaired glucose tolerance, elevated plasma insulin concentrations and insulin resistance, dyslipidemia, hyperglycemia, hyperlipidemia, hypertension, lipodystrophy, cardiovascular disease, respiratory problems or conditions. Diseases or disorders of particular interest are obesity, morbid obesity, morbid obesity prior to surgery, obesity linked inflammation, obesity linked gallbladder disease, obesity induced sleep apnea, hyperlipidemia, dyslipidemia, hypercholesterolemia, atherogenic dyslipidemia, fatty liver disease (FLD), including nonalcoholic fatty liver disease (NAFLD), Non-Alcoholic Steatohepatitis (NASH), insulin resistance, glucose intolerance, pre-diabetes, increased fasting glucose, diabetes mellitus, including type 2 diabetes mellitus, inflammation, including vascular inflammation, hypertension, endothelial dysfunction, dyslipidemia (e.g. high triglycerides, low HDL cholesterol and/or high LDL cholesterol), Prader-Willi Syndrome, insulin resistance, glucose intolerance, pre-diabetes, increased fasting glucose, diabetes mellitus, type 2 diabetes mellitus, inflammation, vascular inflammation, hypertension, endothelial dysfunction, atherosclerosis, arteriosclerosis, coronary heart disease, peripheral artery disease, stroke, or microvascular disease, arterial remodelling, cardiovascular disease (CVD), including atherosclerotic cardiovascular disease, metabolic syndrome (insulin resistance, hypertension, hyperlipidemia), a metabolic disorder, excess body weight, and a combination of any two or more thereof.

In some embodiments, the metabolic disorder is excess body weight (e.g., excess body fat, such as visceral fat, subcutaneous fat, intramuscular fat, and combinations of the foregoing; thus the at least one perturbagen selected from Table 3, or a variant thereof, can be used to promote weight loss, e.g., by reducing excess body fat, such as visceral fat, subcutaneous fat, intramuscular fat, and combinations of the foregoing). In some embodiments, the metabolic disorder is selected from atheromatous disease, atherosclerosis, β-cell dysfunction, cardiovascular disease, coronary heart disease, dyslipidemia, heart disease, hyperglycemia, hypertension, impaired glucose tolerance, an inflammatory disorder, insulin resistance, latent autoimmune diabetes (LADA), metabolic syndrome, nephropathy, neuropathy, nonalcoholic fatty liver disease (NAFLD), nonalcoholic steatohepatitis (NASH), obesity, retinopathy, stroke, Syndrome X, type 1 diabetes and type 2 diabetes. In some embodiments the metabolic disorder is dyslipidemia, atherosclerosis, coronary heart disease, insulin resistance, and type 2 diabetes. In some embodiments the metabolic disorder is dyslipidemia. In some embodiments the metabolic disorder is atherosclerosis. In some embodiments the metabolic disorder is coronary heart disease. In some embodiments the metabolic disorder is insulin resistance. In some embodiments the metabolic disorder is type 2 diabetes. In some embodiments, the disorder is excess body weight (e.g., excess body fat). Excess body fat means excess visceral fat, excess subcutaneous fat, excess intramuscular fat, or combinations of the foregoing. Thus, the at least one perturbagen selected from Table 3, or a variant thereof, can be used to promote weight loss, e.g., by reducing excess body fat, such as visceral fat, subcutaneous fat, intramuscular fat, and combinations of the foregoing). In certain embodiments, the metabolic disorder is not caused (or not driven substantially or not driven principally) by a disorder of protein aggregation, e.g., the dyslipidemia, excess weight, atherosclerosis, coronary heart disease, insulin resistance, obesity, impaired glucose tolerance, atheromatous disease, hypertension, stroke, Syndrome X, heart disease, NASH (non-alcoholic steatohepatitis; as well as related disorders such as NAFLD) (nonalcoholic fatty liver disease), or type 2 diabetes is not driven—solely, principally, or substantially—by a diseased cause by protein aggregates. In other embodiments, the disease may be driven, at least in part, by protein aggregates.

In some embodiments, the metabolic disorder is selected from the group consisting of obesity, diabetes, hypercholesterolemia, hyperlipidemia, nonalcoholic steatohepatitis, and fatty liver.

Glucose Tolerance

In some embodiments, the present disclosure relates to glucose tolerance. The term “glucose tolerance” refers to the ability and time required for the body to respond to the administration of glucose by clearing excess glucose from the circulation. A common test for measuring glucose tolerance is to perform a glucose tolerance test (GTT) on an individual, which typically involves orally administering 75 g of a glucose solution to a fasted individual and measuring blood glucose levels at intervals between 0 and 2 hours after administration. An individual who is “glucose intolerant”, as defined by the World Health Organization, has a 2-hour blood glucose level of between 140 to 199 mg per dL (7.8 to 11.0 mmol/l). An individual who has a 2-hour blood glucose level above 199 mg per dL is also glucose intolerant, but is additionally considered to have type II diabetes. Several different parameters of the GTT may be considered to evaluate the glucose tolerance of an individual, such as the 2-hour blood glucose level, fasting blood glucose level, GTT area under the curve, the max/peak blood glucose concentration obtained by an individual and mean blood glucose level. In some embodiments, improved glucose tolerance is an increase in the amount of glucose tolerance in an individual by at least about 0.5%, and preferably an increase of about 1% or more, e.g. by 5%, 10%, 30%, 50%, 70% or greater of the glucose tolerance of an individual prior to treatment with a compound of the disclosure, or a reduction in the rate that an individual develops glucose intolerance by at least about 1% or more, such as a reduction of glucose tolerance loss by about 5% or more, e.g. by 10%, 30%, 50%, 70%, 90% or greater than the rate of glucose tolerance loss of the individual prior to treatment. Lean mass, blood cholesterol level, blood triglyceride level and glucose tolerance are commonly measured in the fasted state in order to minimize the amount of nutrients which are newly entering circulation from the digestion of food and minimize the amount of nutrients which have yet to be cleared from the blood following ingestion of food.

The term “impaired glucose tolerance” (IGT) or “pre-diabetes” refers to a condition in a person who, when given a glucose tolerance test, has a blood glucose level that falls between normal and hyperglycemic, i.e., has abnormal glucose tolerance, e.g., pathologically abnormal glucose tolerance. Such a person is at a higher risk of developing diabetes although not clinically characterized as having diabetes. In a non-limiting example, impaired glucose tolerance refers to a condition in which a patient has a fasting blood glucose concentration or fasting serum glucose concentration greater than 110 mg/dl and less than 126 mg/dl (7.00 mmol/L), or a 2 hour postprandial blood glucose or serum glucose concentration greater than 140 mg/dl (7.78 mmol/L) and less than 200 mg/dl (11.11 mmol/L). Prediabetes, also referred to as impaired glucose tolerance or impaired fasting glucose is a major risk factor for the development of type 2 diabetes mellitus, cardiovascular disease and mortality.

In some embodiments, the present disclosure provides for methods of treating impaired glucose tolerance in a patient in need thereof. This method includes (a) administering to a patient in need thereof at least one perturbagen selected from Table 3, or a variant thereof, wherein the at least one perturbagen is capable of changing a gene signature in a progenitor cell or (b) administering to a patient in need thereof a cell, the cell having been contacted with at least one perturbagen selected from Table 3, or a variant thereof, wherein the at least one perturbagen is capable of changing a gene signature in a progenitor cell. The at least one perturbagen selected from Table 3, or a variant thereof may be used for manufacture of a medicament for, impaired glucose tolerance, and related conditions.

In some embodiments, the present disclosure provides for methods of promoting improved glucose tolerance in a patient in need thereof. This method includes (a) administering to a patient in need thereof at least one perturbagen selected from Table 3, or a variant thereof, wherein the at least one perturbagen is capable of changing a gene signature in a progenitor cell or (b) administering to a patient in need thereof a cell, the cell having been contacted with at least one perturbagen selected from Table 3, or a variant thereof, wherein the at least one perturbagen is capable of changing a gene signature in a progenitor cell. The at least one perturbagen selected from Table 3, or a variant thereof may be used for manufacture of a medicament for, promoting improved glucose tolerance, and related conditions.

Obesity

In some embodiments, the present disclosure relates to obesity. Obesity is a chronic disease that is highly prevalent in modem society and is associated not only with a social stigma, but also with decreased life span and numerous medical problems, including diabetes mellitus, insulin resistance, hypertension, hypercholesterolemia, cholelithiasis, osteoarthritis, orthopedic injury, thromboembolic disease, cancer, and coronary heart disease. Rissanen et al, British Medical Journal, 301: 835-837 (1990). In some embodiments, Obesity can be calculated using the body mass index (BMI: body weight per height in meters squared). In some embodiments, obesity is defined as an otherwise healthy subject that has a BMI greater than or equal to 30 kg/m², or a condition whereby a subject with at least one co-morbidity has a BMI greater than or equal to 27 kg/m². In some embodiments of the method of the disclosure, the subject is obese and has a body mass index of greater than about 30. In some embodiments, the subject is overweight and has a body mass index of about 25-29.9. In some embodiments, the method induces weight loss. In some embodiments, the method prevents weight gain. In some embodiments, the method prevents the growth of adipose tissue and impair adipocyte differentiation.

As discussed in greater details below, in some embodiments, the present disclosure provides for methods of treatment of obesity in a patient in need thereof. This method includes (a) administering to a patient in need thereof at least one perturbagen selected from Table 3, or a variant thereof, wherein the at least one perturbagen is capable of changing a gene signature in a progenitor cell or (b) administering to a patient in need thereof a cell, the cell having been contacted with at least one perturbagen selected from Table 3, or a variant thereof, wherein the at least one perturbagen is capable of changing a gene signature in a progenitor cell. The at least one perturbagen selected from Table 3, or a variant thereof may be used for manufacture of a medicament for, obesity and overweight, and related conditions. In some embodiments, the present disclosure provides a method for treating or preventing obesity, comprising administering an effective amount of at least one perturbagen selected from Table 3, or a variant thereof to a patient in need thereof. In some aspects, the present disclosure provides a method for weight management, comprising administering an effective amount of at least one perturbagen selected from Table 3, or a variant thereof to induce weight loss and/or to prevent weight gain in.

In some embodiments, the present disclosure relates to a method for inducing weight loss or preventing weight gain (or treating or preventing obesity or inducing weight loss or preventing weight gain in a patient that does not substantially change caloric intake), comprising (a) administering to a patient in need thereof at least one perturbagen selected from Table 3, or a variant thereof, wherein the at least one perturbagen is capable of changing a gene signature in a progenitor cell or (b) administering to a patient in need thereof a cell, the cell having been contacted with at least one perturbagen selected from Table 3, or a variant thereof, wherein the at least one perturbagen is capable of changing a gene signature in a progenitor cell.

In some aspects, the present disclosure provides for uses and methods for inducing weight loss or preventing weight gain, comprising a) administering to a patient in need thereof at least one perturbagen selected from Table 3, or a variant thereof, wherein the at least one perturbagen is capable of changing a gene signature in a progenitor cell or (b) administering to a patient in need thereof a cell, the cell having been contacted with at least one perturbagen selected from Table 3, or a variant thereof, wherein the at least one perturbagen is capable of changing a gene signature in a progenitor cell. In this aspect, the patient may not substantially change caloric intake. In some embodiments, the caloric intake is high, relative to guidelines, such as the USD A tables. In some embodiments, the patient's caloric intake is 2000-10000 calories/day, or greater than about 2000 calories/day, or about 2200 calories/day, or about 2400 calories/day, or about 2600 calories/day, or about 2800 calories/day, or about 3000 calories/day, or about 3200 calories/day, or about 3400 calories/day, or about 3600 calories/day, or about 3800 calories/day, or about 4000 calories/day, or about 5000 calories/day, or about 6000 calories/day. In some embodiments, the patient has a high caloric intake and does not gain weight or even loses weight. Therefore, the present disclosure provides for an effect without life style changes that often reduce patient adherence (e.g., failed dieting). In some embodiments, the patient's caloric intake is not restricted by more than about 20%, or not by more than about 10%, or not by more than about 5% of the patient's caloric intake at the start of treatment. In some embodiments, a high proportion of the patient's caloric intake is “empty calories,” i.e. calories from solid fats and/or added sugars. In some embodiments, greater than about 15%, or 20%, or 25%, or 30%, or 35%, or 50% of the patient's caloric intake is empty calories. Even in these embodiments, a patient may not gain weight or even lose weight.

In some embodiments, the patient of the present disclosure is overweight or obese. In some embodiments, the patient of the present disclosure suffers from central obesity. In some embodiments, the obesity of one of simple obesity (alimentary obesity; usually resulting from consumption of more calories than the body can utilize), secondary obesity (usually resulting from an underlying medical condition, such as, for example, Cushing's syndrome and polycystic ovary syndrome), and childhood obesity. In some embodiments, the obesity is classified as: Class I, which includes a BMI between 30 and 34.99; Class II, which includes BMIs of 35 to 39.99; and Class III, which includes a BMI of over 40. Further, the present disclosure provides for obesity of any of classes I, II, or III that is further classified as severe, morbid, and super obesity. In some embodiments, the patient is at risk of further weight gain, as assessed by, for example, daily caloric intake.

In some embodiments, simple circumferential measurement of the body may be used. In some embodiments, a patient of the present disclosure has a waist circumference exceeding about 35 inches, or about 36 inches, or about 37 inches, or about 38 inches, or about 39 inches, or about 40 inches, or about 41 inches, or about 42 inches, or about 43 inches, or about 44 inches, or about 45 inches, or about 46 inches, or about 47 inches, or about 48 inches, or about 50 inches, or about 55 inches, or about 60 inches. In some embodiments, the patient is male human with a waist circumference exceeding 40 inches. In some embodiments, the patient is a female human with a waist circumference exceeding 35 inches.

The methods of the disclosure may be used to treat humans having a body fat percentage above the recommended body fat percentage, i.e., at least in the “overweight” range, or at least in the “obese” range. The body fat percentage will differ between women and men. Specifically, for women, the methods of the disclosure may be used to treat a female human having a body fat percentage of at least about 25%, above 25%, at least about 32%, or above 32%. For men, the methods of the disclosure may be used to treat a male human having a body fat percentage of at least about 14%, above 14%, at least about 18%, above 18%, at least about 25%, or above 25%. Body fat percentage may be estimated using any method accepted in the art, including, for example, near infrared interactance, dual energy X-ray absorptiometry, body density measurement, bioelectrical impedance analysis, and the like.

The methods of the disclosure may be used to treat a patient who is a man that is greater than 100 pounds' overweight and/or has waist circumference exceeding 40 inches. The methods of the disclosure may be used to treat a patient who is a woman that is greater than 80 pounds' overweight and/or waist circumference exceeding 35 inches.

In some embodiments, the disclosure provides for a at least one perturbagen selected from Table 3, or a variant thereof being used to treat and/or prevent certain disorders associated with being overweight. For example, at least one perturbagen selected from Table 3, or a variant thereof find use in cardiovascular diseases (e.g. high cholesterol, hypercholesterolemia, low HDL, high HDL, hypertension, coronary artery disease, heart failure), sleep apnea (including obstructive sleep apnea), osteoarthritis, thyroid problems, dementia, gout, asthma, gastroesophageal reflux disease, and chronic renal failure.

Diabetes

In some embodiments, the present disclosure relates to diabetes. Diabetes affects many people in the U.S. and many new cases each year. Diabetes is linked to a number of health problems, including microvascular complications, such as retinopathy, neuropathy, and nephropathy. Further, in the United States, diabetes is the leading cause of new blindness in working-age adults, new cases of end-stage renal disease, and non-traumatic lower leg amputations. In addition, cardiovascular complications are now the leading cause of diabetes-related morbidity and mortality, particularly among women and the elderly. In adult patients with diabetes, the risk of cardiovascular disease (CVD) is three-to-five fold greater than in the general population.

As discussed in greater details below, in some embodiments, the present disclosure provides for methods of treatment of diabetes in a patient in need thereof. This method includes (a) administering to a patient in need thereof at least one perturbagen selected from Table 3, or a variant thereof, wherein the at least one perturbagen is capable of changing a gene signature in a progenitor cell or (b) administering to a patient in need thereof a cell, the cell having been contacted with at least one perturbagen selected from Table 3, or a variant thereof, wherein the at least one perturbagen is capable of changing a gene signature in a progenitor cell. The at least one perturbagen selected from Table 3, or a variant thereof may be used for manufacture of a medicament for, diabetes, and related conditions. In some embodiments, the present disclosure provides a method for treating or preventing diabetes, comprising administering an effective amount of at least one perturbagen selected from Table 3, or a variant thereof to a patient in need thereof. In some aspects, the present disclosure provides a method for weight management, comprising administering an effective amount of at least one perturbagen selected from Table 3, or a variant thereof to induce weight loss and/or to prevent weight gain in.

In some aspects, the present technology provides for methods of treatment comprising administering at least one perturbagen selected from Table 3, or a variant thereof in the treatment of or manufacture of a medicament for diabetes and/or glucose intolerance. In some aspects, the present technology provides for a methods treating diabetes, prediabetes, and/or glucose intolerance, comprising administering an effective amount of at least one perturbagen selected from Table 3, or a variant thereof to a patient that suffers from insulin resistance. In some aspects, the present technology provides for methods and uses for preventing diabetes, comprising administering an effective amount of at least one perturbagen selected from Table 3, or a variant thereof to a patient at risk of diabetes, wherein the patient is diagnosed with one or more of insulin resistance, prediabetes, impaired fasting glucose (IFG), impaired glucose tolerance (IGT), and acanthosis nigricans. In some aspects, the present technology provides for a methods treating diabetes, prediabetes, and/or glucose intolerance, comprising administering an effective amount of at least one perturbagen selected from Table 3, or a variant thereof to a patient that suffers from cardiovascular disease or metabolic disease. In some embodiments, the present technology provides for a methods treating diabetes, prediabetes, and/or glucose intolerance, comprising administering an effective amount of at least one perturbagen selected from Table 3, or a variant thereof to a patient that has one or more of an average hemoglobin A1c value of more than about 10% and an average glucose of more than about 200 mg/dl (11 mmol/l) at the start of treatment with conventional diabetic therapy (e.g. insulin therapy and/or non-insulin diabetes agent therapy). In some embodiments, the patient has type 1 diabetes. In some embodiments, the patient has type 2 diabetes. In some embodiments, the patient has gestational diabetes or steroid-induced diabetes. In some embodiments, the at least one perturbagen selected from Table 3, or a variant thereof is administered to a patient that has an average glucose of more than about 200 mg/dl, or more than about 210 mg/dl, or more than about 220 mg/dl, or more than about 230 mg/dl, or more than about 240 mg/dl, or more than about 250 mg/dl at the start of treatment with conventional diabetic therapy. In various embodiments, the conventional diabetic therapy is any one of those described herein, including, for example, insulin therapy and non-insulin diabetes agent therapy.

In some embodiments, at least one perturbagen selected from Table 3, or a variant thereof administration is effective for providing glycemic control. Glycemic control refers to the typical levels of blood sugar (glucose) in a person with diabetes mellitus. Many of the long-term complications of diabetes, including microvascular complications, result from many years of hyperglycemia. Good glycemic control is an important goal of diabetes care. Because blood sugar levels fluctuate throughout the day and glucose records are imperfect indicators of these changes, the percentage of hemoglobin which is glycosylated is used as a proxy measure of long-term glycemic control in research trials and clinical care of people with diabetes. In this test, the hemoglobin Al c or glycosylated hemoglobin reflects average glucose values over the preceding 2-3 months. In non-diabetic persons with normal glucose metabolism glycosylated hemoglobin levels are usually about 4-6% by the most common methods (normal ranges may vary by method). “Perfect glycemic control” indicates that glucose levels are always normal (e.g. about 70-130 mg/dl, or about 3.9-7.2 mmol/L) and indistinguishable from a person without diabetes. In reality, because of the imperfections of treatment measures, even “good glycemic control” describes blood glucose levels that average somewhat higher than normal much of the time. It is noted that what is considered “good glycemic control” varies by age and susceptibility of the patient to hypoglycemia. The

American Diabetes Association has advocated for patients and physicians to strive for average glucose and hemoglobin Al c values below 200 mg/dl (11 mmol/l) and 8%. “Poor glycemic control” refers to persistently elevated blood glucose and glycosylated hemoglobin levels, which may range from, e.g., about 200-500 mg/dl (about 11-28 mmol/L, e.g. about 200 mg/dl, or about 250 mg/dl, or about 300 mg/dl, or about 350 mg/dl, or about 400 mg/dl, or about 450 mg/dl, or about 500 mg/dl) and about 9-15% (e.g.

about 9%, or about 10%, or about 11%, or about 12%, or about 13%, or about 14%, or about 15%) or higher over months and years before severe complications occur. In some aspects, the present technology provides for methods of treatment comprising administering at least one perturbagen selected from Table 3, or a variant thereof and/or uses of at least one perturbagen selected from Table 3, or a variant thereof in the treatment of or manufacture of a medicament for diabetes and/or glucose intolerance. In some embodiments, the present technology provides for a methods treating diabetes, prediabetes, and/or glucose intolerance, comprising administering an effective amount of at least one perturbagen selected from Table 3, or a variant thereof to a patient that suffers from poor glycemic control. In some embodiments, at least one perturbagen selected from Table 3, or a variant thereof administration is effective for providing an average glucose of below about 200 mg/dl (11 mmol/l). In some embodiments, administration of at least one perturbagen selected from Table 3, or a variant thereof is effective for providing an average glucose of below about 190 mg/dl, or about 180 mg/dl, or about 170 mg/dl, or about 160 mg/dl, or about 150 mg/dl, or about 140 mg/dl, or about 130 mg/dl, or about 120 mg/dl, or about 120 mg/dl, or about 110 mg/dl, or about 100 mg/dl, or about 90 mg/dl, or about 80 mg/dl, or about 70 mg/dl. In some embodiments, administration of at least one perturbagen selected from Table 3, or a variant thereof is effective for providing an average glycosylated hemoglobin levels (hemoglobin A1c) values of about 8%, or about 7%, or about 6%, or about 5%, or about 4%. In some embodiments, administration of at least one perturbagen selected from Table 3, or a variant thereof is effective for providing average glycosylated hemoglobin levels (hemoglobin A1c) values of less than about 8%, or less than about 7%, or less than about 6%, or less than about 5%, or less than about 4%.

Administration, Dosing, and Treatment Regimens

As examples, administration results in the delivery of one or more perturbagens disclosed herein into the bloodstream (via enteral or parenteral administration), or alternatively, the one or more perturbagens is administered directly to the site of beige adipocyte proliferation and/or maturation, i.e., in the WAT (e.g.

visceral WAT located inside the peritoneum, and distributed around internal organs, e.g., stomach, liver, intestines, and kidneys). Devices and apparatuses for performing these delivery methods are well known in the art.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any perturbagen disclosed herein as well as the dosing schedule can depend on various parameters and factors, including, but not limited to, the specific perturbagen, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A perturbagen disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, e.g., the white adipose tissue, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

The dosage regimen utilizing any perturbagen disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed. Any perturbagen disclosed herein can be administered in a single daily dose, or the total daily dosage can be administered in divided doses of two, three or four times daily. Furthermore, any perturbagen disclosed herein can be administered continuously rather than intermittently throughout the dosage regimen.

In some embodiments, any perturbagen disclosed herein can be administered in multiple doses. Dosing may be once, twice, three times, four times, five times, six times, or more than six times per day. Dosing may be once a month, once every two weeks, once a week, or once every other day. In some embodiments, a perturbagen disclosed herein is administered about once per day to about 6 times per day. In some embodiments, a perturbagen disclosed herein is administered once daily. In some embodiments, a perturbagen disclosed herein is administered twice daily. In some embodiments, a perturbagen disclosed herein is administered three times daily.

Pharmaceutical Compositions and Formulations

According to another embodiment, the disclosure provides a composition comprising one or more perturbagens, and a pharmaceutically acceptable carrier, adjuvant, or vehicle.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “subject,” as used herein, is used interchangeably with the term “patient” and means an animal, preferably a mammal. In some embodiments, a subject or patient is a human. In other embodiments, a subject (or patient) is a veterinary subject (or patient). In some embodiments, a veterinary subject (or patient) is a canine, a feline, or an equine subject.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride.

Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

Pharmaceutically acceptable compositions of this disclosure can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of one or more perturbagens, it may be desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

One or more perturbagens can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Other Aspects of the Present Technology

Embodiments associated with any of the above-disclosed aspects are likewise relevant to the below-mentioned aspects. In other words, each of the embodiments mentioned above for the above aspects may be revised/adapted to be applicable to the below aspects.

Yet another aspect of the present technology is a use of the perturbagen of Table 3, or a variant thereof in the manufacture of a medicament for treating a disease or disorder characterized by an abnormal ratio of the number of beige adipocytes to the number of one or more of adipoblasts, Myf5− progenitor cells, white preadipocytes, white adipocytes, beige preadipocytes, and/or cells of myogenic lineage selected from the group of Myf5+ progenitor cells, myoblasts, brown preadipocytes, brown adipocytes and skeletal muscle cells.

In an aspect, the present technology provides a use of the perturbagen of Table 3, or a variant thereof in the manufacture of a medicament for treating a disease or disorder characterized by the number of beige adipocytes to the number of white adipocytes.

An aspect of the present technology is a method of identifying a candidate perturbation for promoting the transition of a starting population of progenitor cells comprising an adipocyte stem cell or mesenchymal stem cell into beige adipocytes or immediate progenitors thereof. The method comprises exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into beige adipocytes or immediate progenitors thereof following exposure of the population of cells to the perturbation; and identifying the perturbation as a candidate perturbation for promoting the transition of a population of progenitor cells into beige adipocytes or immediate progenitors thereof based on the perturbation signature, wherein the perturbation signature is an increase in expression and/or activity in the progenitor cell of one or more genes the genes designated as an “up” gene in the gene directionality column of Table 1 and/or a decrease in expression and/or activity in the progenitor cell of one or more genes selected from the genes designated as an “down” gene in the gene directionality column of Table 1.

In some embodiments, the perturbation signature is an increase in expression and/or activity in the progenitor cell of one or more genes selected from in an activation of a network module designated in the network module column of Table 1. In some embodiments, the activation of one or more genes of the network module designated in the network module column of Table 1 comprises modulating expression and/or activity of 2, 3, 4, 5, 6 7, 8, 9, 10 or more genes within a network module. In some embodiments, altering the gene signature comprises a decrease in expression and/or activity in the progenitor cell of one or more genes designated as a “down” gene in the gene directionality column of Table 1. In some embodiments, altering the gene signature comprises a decrease in expression and/or activity in the progenitor cell of two or more genes designated as a “down” gene in the gene directionality column of Table 1.

An aspect of the present technology is a method for making a therapeutic agent for a disease or disorder selected from obesity, morbid obesity, morbid obesity prior to surgery, obesity linked inflammation, obesity linked gallbladder disease, obesity induced sleep apnea, hyperlipidemia, dyslipidemia, hypercholesterolemia, atherogenic dyslipidemia, fatty liver disease (FLD), including nonalcoholic fatty liver disease (NAFLD), Non-Alcoholic Steatohepatitis (NASH), insulin resistance, glucose intolerance, pre-diabetes, increased fasting glucose, diabetes mellitus, including type 2 diabetes mellitus, inflammation, including vascular inflammation, hypertension, endothelial dysfunction, dyslipidemia(e.g. high triglycerides, low HDL cholesterol and/or high LDL cholesterol), Prader-Willi Syndrome, insulin resistance, glucose intolerance, pre-diabetes, increased fasting glucose, diabetes mellitus, type 2 diabetes mellitus, inflammation, vascular inflammation, hypertension, endothelial dysfunction, atherosclerosis, arteriosclerosis, coronary heart disease, peripheral artery disease, stroke, or microvascular disease, arterial remodelling, cardiovascular disease (CVD), including atherosclerotic cardiovascular disease, metabolic syndrome (insulin resistance, hypertension, hyperlipidemia), a metabolic disorder, excess body weight, and a combination of any two or more thereof. The method comprises (a) identifying a therapeutic agent for therapy according to any of the embodiments disclosed herein, and (b) formulating the candidate perturbation as a therapeutic agent for the treatment of the disease or disorder. In this aspect, identifying a therapeutic agent for therapy comprises steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell fate of the population of the population of progenitor cells into beige adipocytes or immediate progenitors thereof following exposure of the population of cells to the perturbation; and identifying the perturbation as a candidate perturbation for promoting the transition of a population of progenitor cells into beige adipocytes or immediate progenitors thereof based on the perturbation signature. Further, in this aspect, the perturbation signature is an increase in expression and/or activity in the progenitor cell of one or more genes selected from in an activation of a network module designated in the network module column of Table 1. In some embodiments, the activation of one or more genes of the network module designated in the network module column of Table 1 comprises modulating expression and/or activity of 2, 3, 4, 5, 6 7, 8, 9, 10 or more genes within a network module. In some embodiments, altering the gene signature comprises a decrease in expression and/or activity in the progenitor cell of one or more genes designated as a “down” gene in the gene directionality column of

Table 1. In some embodiments, altering the gene signature comprises a decrease in expression and/or activity in the progenitor cell of two or more genes designated as a “down” gene in the gene directionality column of Table 1.

Yet another aspect of the present technology is a perturbagen capable of causing a change in a gene signature.

In an aspect, the present technology provides a perturbagen capable of causing a change in cell fate.

In another aspect, the present technology provides a perturbagen capable of causing a change in a gene signature and a change in cell fate.

In yet another aspect, the present technology provides a pharmaceutical composition comprising any herein disclosed perturbagen.

In a further aspect, the present technology provides a unit dosage form comprising an effective amount of the pharmaceutical composition comprising any herein disclosed perturbagen.

The instant disclosure also provides certain embodiments as follows:

Embodiment 1: A method for directing a change in cell state of a progenitor cell comprising: contacting a population of cells comprising a progenitor cell with at least one perturbagen selected from

Table 3, or a variant thereof, wherein the at least one perturbagen is capable of altering a gene signature in the progenitor cell; and wherein the progenitor cell is an adipocyte stem cell or a mesenchymal stem cell.

Embodiment 2: A method for directing a change in cell state of a progenitor cell, comprising: contacting a population of cells comprising a progenitor cell with at least one perturbagen capable of altering a gene signature in the progenitor cell, wherein altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1 and wherein the progenitor cell is an adipocyte stem cell or a mesenchymal stem cell.

Embodiment 3: A method for directing a change in cell state of a progenitor cell, comprising: contacting a population of cells comprising a progenitor cell with at least one perturbagen selected from Table 3, or a variant thereof, and capable of altering a gene signature in the progenitor cell; wherein altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1; and wherein the progenitor cell is an adipocyte stem cell or a mesenchymal stem cell.

Embodiment 4: The method of any one of Embodiments 1-3, wherein altering the gene signature comprises a change in expression and/or activity of one or more genes in the progenitor cell of a network module designated in the network module column of Table 1.

Embodiment 5: The method of any one of Embodiments 1-4, wherein the activation of one or more genes of the network module designated in the network module column of Table 1 comprises modulating expression and/or activity of 2 or more genes within a network module.

Embodiment 6: The method of any one of Embodiments 1-5, wherein altering the gene signature comprises an increase in expression and/or activity in the progenitor cell of two or more genes designated as an “up” gene in the gene directionality column of Table 1.

Embodiment 7: The method of any one of Embodiments 1-6, wherein altering the gene signature comprises a decrease in expression and/or activity in the progenitor cell of two or more genes designated as a “down” gene in the gene directionality column of Table 1.

Embodiment 8: The method of any one of Embodiments 1-7, wherein the change in cell state provides an increase in the number of one or more of adipoblasts, Myf5− progenitor cells, beige preadipocytes, and/or beige adipocytes.

Embodiment 9: The method of Embodiment 8, wherein the increase in the number of adipoblasts, Myf5− progenitor cells, beige preadipocytes, and/or beige adipocytes is relative to the number of adipoblasts, Myf5− progenitor cells, beige preadipocytes, and/or beige adipocytes obtained from a population of progenitor cells that are not contacted with the at least one perturbagen.

Embodiment 10: The method of Embodiment 8 or Embodiment 9, wherein the increase in the number of adipoblasts, Myf5− progenitor cells, beige preadipocytes, and/or beige adipocytes is relative to the number of adipoblasts, Myf5− progenitor cells, beige preadipocytes, and/or beige adipocytes obtained from a population of progenitor cells prior to contacting with the at least one perturbagen.

Embodiment 11: The method of any one of Embodiments 1-10, wherein the change in cell state provides an increase in the number of beige adipocytes.

Embodiment 12: The method of Embodiment 11, wherein the increase in the number of beige adipocytes is relative to the number of beige adipocytes obtained from a population of progenitor cells that are not contacted with the at least one perturbagen.

Embodiment 13: The method of Embodiment 11 or Embodiment 12, wherein the increase in the number of beige adipocytes is relative to the number of beige adipocytes obtained from a population of progenitor cells prior to contacting with the at least one perturbagen.

Embodiment 14: The method of any one of Embodiments 1-13, wherein the change in cell state provides a substantial increase in the number of beige preadipocytes and/or beige adipocytes.

Embodiment 15: The method of Embodiment 14, wherein the substantial increase in the number of beige preadipocytes and/or beige adipocytes is relative to the number of beige preadipocytes and/or beige adipocytes obtained from a population of progenitor cells that are not contacted with the at least one perturbagen.

Embodiment 16: The method of Embodiment 14 or Embodiment 15, wherein the substantial increase in the number of beige preadipocytes and/or beige adipocytes is relative to the number of beige preadipocytes and/or beige adipocytes obtained from a population of progenitor cells prior to contacting with the at least one perturbagen.

Embodiment 17: The method of any one of Embodiments 1-16, wherein the change in cell state does not provide a substantial increase in the number of white preadipocytes and/or white adipocytes.

Embodiment 18: The method of any one of Embodiments 1-13, wherein the change in cell state provides a substantial decrease in the number of cells of white preadipocytes and/or white adipocytes.

Embodiment 19: The method of Embodiment 18, wherein the substantial decrease in the number of preadipocytes and/or white adipocytes is relative to the number of preadipocytes and/or white adipocytes obtained from a population of progenitor cells that are not contacted with the at least one perturbagen.

Embodiment 20: The method of Embodiment 18, wherein the substantial decrease in the number of preadipocytes and/or white adipocytes is relative to the number of preadipocytes and/or white adipocytes obtained from a population of progenitor cells prior to contacting with the at least one perturbagen.

Embodiment 21: The method of any one of Embodiments 1-20, wherein the change in cell state provides a substantial increase in the number of other committed cells of myogenic lineage selected from the group of Myf5+ progenitor cells, myoblasts, brown preadipocytes, brown adipocytes and skeletal muscle cells.

Embodiment 22: The method of any one of Embodiments 1-20, wherein the change in cell state does not provide a substantial increase in the number of other committed cells of myogenic lineage selected from the group of Myf5+ progenitor cells, myoblasts, brown preadipocytes, brown adipocytes and skeletal muscle cells.

Embodiment 23: The method of Embodiment 21 or Embodiment 22, wherein the substantial increase in the number of other committed cells of myogenic lineage selected from the group of Myf5+ progenitor cells, myoblasts, brown preadipocytes, brown adipocytes and skeletal muscle cells is relative to the number of other committed cells of myogenic lineage selected from the group of Myf5+ progenitor cells, myoblasts, brown preadipocytes, brown adipocytes and skeletal muscle cells obtained from a population of progenitor cells that are not contacted with the at least one perturbagen.

Embodiment 24: The method of Embodiment 21 or Embodiment 22, wherein the substantial increase in the number of other committed cells of myogenic lineage selected from the group of Myf5+ progenitor cells, myoblasts, brown preadipocytes, brown adipocytes and skeletal muscle cells is relative to the number of other committed cells of myogenic lineage selected from the group of Myf5+ progenitor cells, myoblasts, brown preadipocytes, brown adipocytes and skeletal muscle cells obtained from a population of progenitor cells prior to contacting with the at least one perturbagen.

Embodiment 25: The method of any one of Embodiments 1-20, wherein the change in cell state provides a substantial decrease in the number of other committed cells of myogenic lineage selected from the group of Myf5+ progenitor cells, myoblasts, brown preadipocytes, brown adipocytes and skeletal muscle cells.

Embodiment 26: The method of Embodiment 25, wherein the substantial decrease in the number of other committed cells of myogenic lineage selected from the group of Myf5+ progenitor cells, myoblasts, brown preadipocytes, brown adipocytes and skeletal muscle cells is relative to the number of other committed cells of myogenic lineage selected from the group of Myf5+ progenitor cells, myoblasts, brown preadipocytes, brown adipocytes and skeletal muscle cells obtained from a population of progenitor cells that are not contacted with the at least one perturbagen.

Embodiment 27: The method of Embodiment 25 or Embodiment 26, wherein the substantial decrease in the number of other committed cells of myogenic lineage selected from the group of Myf5+ progenitor cells, myoblasts, brown preadipocytes, brown adipocytes and skeletal muscle cells is relative to the number of other committed cells of myogenic lineage selected from the group of Myf5+ progenitor cells, myoblasts, brown preadipocytes, brown adipocytes and skeletal muscle cells obtained from a population of progenitor cells prior to contacting with the at least one perturbagen.

Embodiment 28: The method of any one of Embodiments 1-27, wherein the increase in the number of adipoblasts, Myf5− progenitor cells, beige preadipocytes, and/or beige adipocytes is due in part to decreased cell proliferation of other committed cells of myogenic lineage selected from the group of Myf5+ progenitor cells, myoblasts, brown preadipocytes, brown adipocytes and skeletal muscle cells.

Embodiment 29: The method of any one of Embodiments 1-27, wherein the increase in the number of adipoblasts, Myf5− progenitor cells, beige preadipocytes, and/or beige adipocytes is due in part to increased survival of other committed cells of myogenic lineage selected from the group of Myf5+ progenitor cells, myoblasts, brown preadipocytes, brown adipocytes and skeletal muscle cells.

Embodiment 30: The method of any one of Embodiments 1-29, wherein the increase in the number of adipoblasts, Myf5− progenitor cells, beige preadipocytes, and/or beige adipocytes is due in part to an increased lifespan of the adipoblasts, Myf5− progenitor cells, beige preadipocytes, and/or beige adipocytes.

Embodiment 31: The method of any one of Embodiments 1-30, wherein the increase in the number of adipoblasts, Myf5− progenitor cells, beige preadipocytes, and/or beige adipocytes is due in part to a decreased cell death of the adipoblasts, Myf5− progenitor cells, beige preadipocytes, and/or beige adipocytes.

Embodiment 32: The method of any one of Embodiments 1-31, wherein the increase in the number of beige adipocytes is due in part to an increased lifespan of the beige adipocytes.

Embodiment 33: The method of any one of Embodiments 1-32, wherein the increase in the number of beige adipocytes is due in part to a decreased cell death of the beige adipocytes.

Embodiment 34: The method of any one of Embodiments 1-33, wherein the increase in the number of beige adipocytes is due in part to a decreased apoptosis of the beige adipocytes.

Embodiment 35: The method of any one of Embodiments 1-34, wherein the increase in the number of beige adipocytes is due in part to decreased lifespan of white preadipocytes and/or white adipocytes.

Embodiment 36: The method of any one of Embodiments 1-35, wherein the increase in the number of beige adipocytes is due in part to increased cell death of white preadipocytes and/or white adipocytes.

Embodiment 37: The method of any one of Embodiments 1-36, wherein the increase in the number of beige adipocytes is due in part to increased apoptosis of white preadipocytes and/or white adipocytes.

Embodiment 38: The method of any one of Embodiments 1-34, wherein the increase in the number of beige adipocytes is due in part to decreased lifespan of white adipocytes.

Embodiment 39: The method of any one of Embodiments 1-34 or 38, wherein the increase in the number of beige adipocytes is due in part to increased cell death of white adipocytes.

Embodiment 40: The method of any one of Embodiments 1-34 or 38-39, wherein the increase in the number of beige adipocytes is due in part to increased apoptosis of white adipocytes.

Embodiment 41:The method of any one of Embodiments 1-40, wherein the increase in the number of beige adipocytes is due in part to increased transdifferentiation of white adipocytes to beige preadipocytes.

Embodiment 42: The method of any one of Embodiments 1-41, wherein the increase in the number of beige adipocytes is due in part to increased lifespan of beige preadipocytes.

Embodiment 43: The method of any one of Embodiments 1-42, wherein the increase in the number of beige adipocytes is due in part to decreased cell death of beige preadipocytes.

Embodiment 44: The method of any one of Embodiments 1-43, wherein the increase in the number of beige adipocytes is due in part to decreased apoptosis of beige preadipocytes.

Embodiment 45: The method of any one of Embodiments 1-44, wherein the increase in the number of beige adipocytes is due in part to increased differentiation of beige preadipocytes to beige adipocytes.

Embodiment 46: The method of any one of Embodiments 1-45, wherein the increase in the number of beige adipocytes is due in part to increased lifespan of Myf5− progenitor cells.

Embodiment 47: The method of any one of Embodiments 1-46, wherein the increase in the number of beige adipocytes is due in part to decreased cell death of Myf5− progenitor cells.

Embodiment 48: The method of any one of Embodiments 1-47, wherein the increase in the number of beige adipocytes is due in part to decreased apoptosis of Myf5− progenitor cells.

Embodiment 49: The method of any one of Embodiments 1-48, wherein the increase in the number of beige adipocytes is due in part to increased differentiation of Myf5− progenitor cells.

Embodiment 50:The method of any one of Embodiments 1-49, wherein the increase in the number of beige adipocytes is due in part to increased lifespan of adipoblasts.

Embodiment 51: The method of any one of Embodiments 1-50, wherein the increase in the number of beige adipocytes is due in part to decreased cell death of adipoblasts.

Embodiment 52: The method of any one of Embodiments 1-51, wherein the increase in the number of beige adipocytes is due in part to decreased apoptosis of adipoblasts.

Embodiment 53: The method of any one of Embodiments 1-52, wherein the increase in the number of beige adipocytes is due in part to increased differentiation of adipoblasts.

Embodiment 54: The method of any one of Embodiments 1-53, wherein the increase in the number of beige adipocytes is due in part to increased lifespan of mesenchymal stem cells.

Embodiment 55: The method of any one of Embodiments 1-54, wherein the increase in the number of beige adipocytes is due in part to decreased cell death of mesenchymal stem cells.

Embodiment 56: The method of any one of Embodiments 1-55, wherein the increase in the number of beige adipocytes is due in part to decreased apoptosis of mesenchymal stem cells.

Embodiment 57 : The method of any one of Embodiments 1-56, wherein the increase in the number of beige adipocytes is due in part to increased differentiation of mesenchymal stem cells.

Embodiment 58: The method of any one of Embodiments 1-57, wherein the increase in the number of beige adipocytes is due in part to decreased cell death of other committed cells of myogenic lineage selected from the group of Myf5+ progenitor cells, myoblasts, brown preadipocytes, brown adipocytes and skeletal muscle cells.

Embodiment 59: The method of any one of Embodiments 1-58, wherein the increase in the number of beige adipocytes is due in part to decreased apoptosis of other committed cells of myogenic lineage selected from the group of Myf5+ progenitor cells, myoblasts, brown preadipocytes, brown adipocytes and skeletal muscle cells.

Embodiment 60: The method of any one of Embodiments 1-57, wherein the increase in the number of beige adipocytes is due in part to increased cell death of other committed cells of myogenic lineage selected from the group of Myf5+ progenitor cells, myoblasts, brown preadipocytes, brown adipocytes and skeletal muscle cells.

Embodiment 61: The method of any one of Embodiments 1-57 or 60, wherein the increase in the number of beige adipocytes is due in part to increased apoptosis of other committed cells of myogenic lineage selected from the group of Myf5+ progenitor cells, myoblasts, brown preadipocytes, brown adipocytes and skeletal muscle cells.

Embodiment 62: The method of any one of Embodiments 1-61, wherein the increase in the number of beige adipocytes is due in part to a change of cell state from mesenchymal stem cells into committed cells of myogenic lineage selected from the group of Myf5+ progenitor cells, myoblasts, brown preadipocytes, brown adipocytes and skeletal muscle cells; and/or adipoblasts, Myf5− progenitor cells, beige preadipocytes, and/or beige adipocytes.

Embodiment 63: The method of any one of Embodiments 1-62, wherein the increase in the number of beige adipocytes is due in part to a change of cell state from mesenchymal stem cells into adipoblasts, Myf5− progenitor cells, beige preadipocytes, and/or beige adipocytes.

Embodiment 64: The method of any one of Embodiments 1-63, wherein the number of mesenchymal stem cells is decreased.

Embodiment 65: The method of Embodiment 64, wherein the decrease in the number of mesenchymal stem cells is due in part to decreased cell proliferation of the mesenchymal stem cells.

Embodiment 66: The method of Embodiment 64 or Embodiment 65, wherein the decrease in the number of mesenchymal stem cells is due in part to a decreased lifespan of the mesenchymal stem cells.

Embodiment 67: The method of any one of Embodiments 64-66, wherein the decrease in the number of mesenchymal stem cells is due in part to an increased cell death of the mesenchymal stem cells.

Embodiment 68: The method of any one of Embodiments 64-67, wherein the decrease in the number of mesenchymal stem cells is due in part to an increased apoptosis of the mesenchymal stem cells.

Embodiment 69: The method of any one of Embodiments 64-68, wherein the decrease in the number of mesenchymal stem cells is relative to the number of mesenchymal stem cells in a population of progenitor cells that are not contacted with the at least one perturbagen.

Embodiment 70: The method of any one of Embodiments 64-69, wherein the decrease in the number of mesenchymal stem cells is relative to the number of mesenchymal stem cells in a population of progenitor cells prior to contacting with the at least one perturbagen.

Embodiment 71: The method of any one of Embodiments 64-70, wherein the decrease in the number of mesenchymal stem cells is due in part to an increased differentiation of the mesenchymal stem cells.

Embodiment 72: The method of any one of Embodiments 64-71, wherein the decrease in the number of mesenchymal stem cells is due to a change of cell state from a progenitor cell into the committed cells of myogenic lineage selected from the group of Myf5+ progenitor cells, myoblasts, brown preadipocytes, brown adipocytes and skeletal muscle cells; and adipoblasts, Myf5− progenitor cells, beige preadipocytes, and/or beige adipocytes.

Embodiment 73: The method of any one of Embodiments 1-63, wherein the number of mesenchymal stem cells is increased.

Embodiment 74: The method of Embodiment 73, wherein the increase in the number of mesenchymal stem cells is due in part to increased cell proliferation of the mesenchymal stem cells.

Embodiment 75: The method of Embodiment 73 or Embodiment 74, wherein the increase in the number of mesenchymal stem cells is due in part to an increased lifespan of the mesenchymal stem cells.

Embodiment 76: The method of any one of Embodiments 73-75, wherein the increase in the number of mesenchymal stem cells is due in part to decreased cell death among the mesenchymal stem cells.

Embodiment 77: The method of any one of Embodiments 73-76, wherein the increase in the number of mesenchymal stem cells is relative to the number of mesenchymal stem cells in a population of progenitor cells that are not contacted with the at least one perturbagen.

Embodiment 78: The method of any one of Embodiments 73-77, wherein the increase in the number of mesenchymal stem cells is relative to the number of mesenchymal stem cells in a population of progenitor cells prior to contacting with the at least one perturbagen.

Embodiment 79: The method of any one of Embodiments 1-78, wherein the number of beige adipocytes is increased after contacting the population of progenitor cells comprising an adipocyte stem cell or mesenchymal stem cell with the at least one perturbagen.

Embodiment 80: The method of any one of Embodiments 1-79, wherein the number of beige preadipocytes, and/or beige adipocytes is increased after contacting the population of progenitor cells comprising an adipocyte stem cell or mesenchymal stem cell with the at least one perturbagen.

Embodiment 81: The method of any one of Embodiments 1-80, wherein the number of beige preadipocytes, and/or beige adipocytes is increased after contacting the population of progenitor cells comprising an adipocyte stem cell or mesenchymal stem cell with the at least one perturbagen.

Embodiment 82: The method of any one of Embodiments 1-81, wherein the number of Myf5− progenitors cells, beige preadipocytes, and/or beige adipocytes is increased after contacting the population of progenitor cells comprising an adipocyte stem cell or mesenchymal stem cell with the at least one perturbagen.

Embodiment 83: The method of any one of Embodiments 1-82, wherein the number of adipoblasts, Myf5− progenitor cells, beige preadipocytes, and/or beige adipocytes is increased after contacting the population of progenitor cells comprising an adipocyte stem cell or mesenchymal stem cell with the at least one perturbagen.

Embodiment 84: The method of any one of Embodiments 1-83, wherein the number of committed cells of myogenic lineage selected from the group of Myf5+ progenitor cells, myoblasts, brown preadipocytes, brown adipocytes and/or skeletal muscle cells is increased after contacting the population of cells comprising a adipocyte stem cell or mesenchymal stem cell with the at least one perturbagen.

Embodiment 85: The method of any one of Embodiments 1-83, wherein the number of committed cells of myogenic lineage selected from the group of Myf5+ progenitor cells, myoblasts, brown preadipocytes, brown adipocytes and/or skeletal muscle cells is decreased after contacting the population of cells comprising a adipocyte stem cell or mesenchymal stem cell with the at least one perturbagen.

Embodiment 86: The method of any one of Embodiments 1-85, wherein the ratio of the number of beige adipocytes to the number of white adipocytes is increased relative to the ratio in the population of progenitor cells prior to contacting with the at least one perturbagen.

Embodiment 87: The method of any one of Embodiments 1-86, wherein the ratio of the number of beige adipocytes to the number of white preadipocytes is increased relative to the ratio in the population of progenitor cells prior to contacting with the at least one perturbagen.

Embodiment 88: The method of any one of Embodiments 1-87, wherein the ratio of the number of beige adipocytes to the number of Myf5− progenitors is increased relative to the ratio in the population of progenitor cells that are not contacted with the at least one perturbagen.

Embodiment 89: The method of any one of Embodiments 1-88, wherein the ratio of the number of beige adipocytes to the number of adipoblasts is increased relative to the ratio in the population of progenitor cells that are not contacted with the at least one perturbagen.

Embodiment 90: The method of any one of Embodiments 1-89, wherein the ratio of the number of the beige adipocytes to the number of the committed cells of myogenic lineage selected from the group of Myf5+ progenitor cells, myoblasts, brown preadipocytes, brown adipocytes and/or skeletal muscle cells is increased relative to the ratio in the population of progenitor cells that are not contacted with the at least one perturbagen.

Embodiment 91: The method of any one of Embodiments 1-90, wherein the ratio of the number of white adipocytes to the number of white preadipocytes is decreased relative to the ratio in the population of progenitor cells prior to contacting with the at least one perturbagen.

Embodiment 92: The method of any one of Embodiments 1-91, wherein the ratio of the number of white adipocytes to the number of Myf5− progenitors is decreased relative to the ratio in the population of progenitor cells prior to contacting with the at least one perturbagen.

Embodiment 93: The method of any one of Embodiments 1-92, wherein the ratio of the number of white adipocytes to the number of adipoblasts is decreased relative to the ratio in the population of progenitor cells prior to contacting with the at least one perturbagen.

Embodiment 94: The method of any one of Embodiments 1-93, wherein the ratio of the number of white adipocytes to the number of mesenchymal stem cells is decreased relative to the ratio in the population of progenitor cells prior to contacting with the at least one perturbagen.

Embodiment 95: The method of any one of Embodiments 1-94, wherein the ratio of the number of beige preadipocytes to the number of Myf5− progenitors is decreased relative to the ratio in the population of progenitor cells prior to contacting with the at least one perturbagen.

Embodiment 96: The method of any one of Embodiments 1-95, wherein the ratio of the number of beige preadipocytes to the number of adipoblasts is increased relative to the ratio in the population of progenitor cells prior to contacting with the at least one perturbagen.

Embodiment 97: The method of any one of Embodiments 1-96, wherein the ratio of the number of beige preadipocytes to the number of mesenchymal stem cells is increased relative to the ratio in the population of progenitor cells prior to contacting with the at least one perturbagen.

Embodiment 98: The method of any one of Embodiments 1-97, wherein the ratio of the number of Myf5− progenitors to the number of adipoblasts is increased relative to the ratio in the population of progenitor cells prior to contacting with the at least one perturbagen.

Embodiment 99: The method of any one of Embodiments 1-98, wherein the ratio of the number of Myf5− progenitors to the number of mesenchymal stem cells is increased relative to the ratio in the population of progenitor cells prior to contacting with the at least one perturbagen.

Embodiment 100: The method of any one of Embodiments 1-99, wherein the ratio of the number of adipoblasts to the number of mesenchymal stem cells is increased relative to the ratio in the population of progenitor cells prior to contacting with the at least one perturbagen.

Embodiment 101: The method of any one of Embodiments 1-100, wherein the ratio of the number of beige adipocytes to the number of white adipocytes is increased relative to the ratio in the population of progenitor cells that is not contacted with the at least one perturbagen.

Embodiment 102: The method of any one of Embodiments 1-101, wherein the ratio of the number of beige adipocytes to the number of white preadipocytes is increased relative to the ratio in the population of progenitor cells that is not contacted with the at least one perturbagen.

Embodiment 103: The method of any one of Embodiments 1-102, wherein the ratio of the number of beige adipocytes to the number of Myf5− progenitors is increased relative to the ratio in the population of progenitor cells that are not contacted with the at least one perturbagen.

Embodiment 104: The method of any one of Embodiments 1-103, wherein the ratio of the number of beige adipocytes to the number of adipoblasts is increased relative to the ratio in the population of progenitor cells that are not contacted with the at least one perturbagen.

Embodiment 105: The method of any one of Embodiments 1-104, wherein the ratio of the number of the beige adipocytes to the number of the committed cells of myogenic lineage selected from the group of Myf5+ progenitor cells, myoblasts, brown preadipocytes, brown adipocytes and/or skeletal muscle cells is increased relative to the ratio in the population of progenitor cells that are not contacted with the at least one perturbagen.

Embodiment 106: The method of any one of Embodiments 1-105, wherein the ratio of the number of white adipocytes to the number of white preadipocytes is decreased relative to the ratio in the population of progenitor cells that is not contacted with the at least one perturbagen.

Embodiment 107: The method of any one of Embodiments 1-106, wherein the ratio of the number of white adipocytes to the number of Myf5− progenitors is decreased relative to the ratio in the population of progenitor cells that is not contacted with the at least one perturbagen.

Embodiment 108: The method of any one of Embodiments 1-107, wherein the ratio of the number of white adipocytes to the number of adipoblasts is decreased relative to the ratio in the population of progenitor cells that is not contacted with the at least one perturbagen.

Embodiment 109: The method of any one of Embodiments 1-108, wherein the ratio of the number of white adipocytes to the number of mesenchymal stem cells is decreased relative to the ratio in the population of progenitor cells that is not contacted with the at least one perturbagen.

Embodiment 110: The method of any one of Embodiments 1-109, wherein the ratio of the number of beige preadipocytes to the number of Myf5− progenitors is increased relative to the ratio in the population of progenitor cells that is not contacted with the at least one perturbagen.

Embodiment 111: The method of any one of Embodiments 1-110, wherein the ratio of the number of beige preadipocytes to the number of adipoblasts is increased relative to the ratio in the population of progenitor cells that is not contacted with the at least one perturbagen.

Embodiment 112: The method of any one of Embodiments 1-111, wherein the ratio of the number of beige preadipocytes to the number of mesenchymal stem cells is increased relative to the ratio in the population of progenitor cells that is not contacted with the at least one perturbagen.

Embodiment 113: The method of any one of Embodiments 1-112, wherein the ratio of the number of Myf5− progenitors to the number of adipoblasts is increased relative to the ratio in the population of progenitor cells that is not contacted with the at least one perturbagen.

Embodiment 114: The method of any one of Embodiments 1-113, wherein the ratio of the number of Myf5− progenitors to the number of mesenchymal stem cells is increased relative to the ratio in the population of progenitor cells that is not contacted with the at least one perturbagen.

Embodiment 115: The method of any one of Embodiments 1-114, wherein the ratio of the number of adipoblasts to the number of mesenchymal stem cells is increased relative to the ratio in the population of progenitor cells that is not contacted with the at least one perturbagen.

Embodiment 116: The method of any one of Embodiments 1-115, wherein the number of adipoblasts is increased relative to the number of adipoblasts in the population of progenitor cells prior to contacting with the at least one perturbagen.

Embodiment 117: The method of any one of Embodiments 1-116, wherein the number of adipoblasts is increased relative to the number of adipoblasts in the population of progenitor cells that is not contacted with the at least one perturbagen.

Embodiment 118: The method of any one of Embodiments 1-117, wherein the number of Myf5− progenitors is increased relative to the number of Myf5− progenitors in the population of progenitor cells prior to contacting with the at least one perturbagen.

Embodiment 119: The method of any one of Embodiments 1-118, wherein the number of Myf5− progenitors is increased relative to the number of Myf5− progenitors in the population of progenitor cells that is not contacted with the at least one perturbagen.

Embodiment 120: The method of any one of Embodiments 1-119, wherein the number of white preadipocytes is increased relative to the number of white preadipocytes in the population of progenitor cells prior to contacting with the at least one perturbagen.

Embodiment 121: The method of any one of Embodiments 1-120, wherein the number of white preadipocytes is decreased relative to the number of white preadipocytes in the population of progenitor cells that is not contacted with the at least one perturbagen.

Embodiment 122: The method of any one of Embodiments 1-121, wherein the number of white adipocytes is decreased relative to the number of white adipocytes in the population of progenitor cells prior to contacting with the at least one perturbagen.

Embodiment 123: The method of any one of Embodiments 1-122, wherein the number of white adipocytes is increased relative to the number of white adipocytes in the population of progenitor cells that is not contacted with the at least one perturbagen.

Embodiment 124: The method of any one of Embodiments 1-123, wherein the number of beige preadipocytes is increased relative to the number of beige preadipocytes in the population of progenitor cells prior to contacting with the at least one perturbagen.

Embodiment 125: The method of any one of Embodiments 1-124, wherein the number of beige preadipocytes is increased relative to the number of beige preadipocytes in the population of progenitor cells that is not contacted with the at least one perturbagen.

Embodiment 126: The method of any one of Embodiments 1-125, wherein the at least one perturbagen is selected from Table 3, or a variant thereof, comprises at least 2, at least 3, at least 4, or at least 5 perturbagens selected from Table 3, or variants thereof.

Embodiment 127: The method of any one of Embodiments 2-126, wherein the one or more genes are selected from the genes designated as an “up” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, or 55 genes selected from the genes designated as an “up” gene in the gene directionality column of Table 1.

Embodiment 128: The method of any one of Embodiments 2-127, wherein the one or more genes are selected from MRPL12, MIF, BIRC5, CDK1, UBE2C, CISD1, TOMM70A, RRS1, TRAP1, DLD, GAPDH, CDK4, MRPS16, GALE, CAT, PFKL, CEBPA, SCP2, LPGAT1, NT5DC2, GNB5, LAP3, HSPA4, PPARG, HSPD1, NOLC1, SDHB, PGAM1, TIMM9, CCNB2, IFRD2, MPC2, STMN1, PARP1, UBE2A, GSTZ1, SCARB1, HADH, PEX11A, ETFB, HSD17B10, PXMP2, CIAPIN1, DNAJC15, FKBP4, SMARCA4, BZW2, VDAC1, ISOC1, CYCS, G3BP1, CD320, MYLK, EIF4EBP1, and/or PHGDH.

Embodiment 129: The method of any one of Embodiments 2-128, wherein the one or more genes are selected from the genes designated as an “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, or 73 genes selected from the genes designated as an “down” gene in the gene directionality column of Table 1.

Embodiment 130: The method of any one of Embodiments 2-129, wherein the one or more genes are selected from JUN, 1D2, ZFP36, BAMBI, HERPUD1, NCK1, MYC, SQSTM1, NFKBIA, IER3, TIPARP, HES1, CYB561, HSD17B11, FYN, PLSCR1, CIRBP, FOS, NUCB2, S100A13, RSU1, ASAH1, TIMP2, COL1A1, MYL9, LOXL1, COL4A1, DNM1, MMP2, PTGS2, SSBP2, RTN2, VAT1, GAA, PROS1, B4GAT1, PNP, DRAP1, PRSS23, IGFBP3, TPM1, ILK, SCRN1, FHL2, KDM5B, GADD45B, EGFR, GRN, SERPINE1, TMEM50A, CLTB, GNA12, FKBP14, CALU, STXBP1, HTRA1, UGDH, PLA2G4A, EXT1, CTSL, NENF, TGFBR2, EGR1, P4HA2, GNAS, APP, HSPA1A, ITGB5, EPB41L2, GSTM2, ICAM1, CEBPD, and/or CHIC2.

Embodiment 131: The method of any one of Embodiments 1-130, wherein the contacting the population of progenitor cells occurs in vitro or ex vivo.

Embodiment 132: The method of any one of Embodiments 1-131, wherein the contacting the population of progenitor cells occurs in vivo in a subject.

Embodiment 133: The method of Embodiment 132, wherein the subject is a human.

Embodiment 134: The method of Embodiment 132 or Embodiment 133, wherein the subject is an adult human.

Embodiment 135: The method of any one of Embodiments 1-134, wherein the method provides an increase in expression and/or activity of uncoupling protein 1 (UCP1), optionally as compared to the expression and/or activity the absence of a perturbagen.

Embodiment 136: The method of any one of Embodiments 1-135, wherein the method provides an increase in expression and/or activity of uncoupling protein 1 (UCP1), optionally as compared to the expression and/or activity prior to contacting with the at least one perturbagen.

Embodiment 137: The method of any one of Embodiments 1-136, wherein the method increases the amount or extent of non-shivering thermogenesis, optionally as compared to the amount or extent of non-shivering thermogenesis to the absence of a perturbagen.

Embodiment 138: The method of any one of Embodiments 1-137, wherein the method increases the amount or extent of non-shivering thermogenesis, optionally as compared to the amount or extent of non-shivering thermogenesis prior to contacting with the at least one perturbagen.

Embodiment 139: The method of any one of Embodiments 1-138, wherein the method increases or stimulates the beiging and/or browning of white adipose tissue (WAT), optionally as compared to the beiging and/or browning of WAT in the absence of a perturbagen.

Embodiment 140: The method of any one of Embodiments 1-139, wherein the method increases or stimulates the beiging and/or browning of white adipose tissue (WAT), optionally as compared to the beiging and/or browning of WAT prior to contacting with the at least one perturbagen.

Embodiment 141: The method of any one of Embodiments 1-140, wherein the method enhances energy expenditure by reducing lipids stored within adipose tissue.

Embodiment 142: The method of any one of Embodiments 1-141, wherein the method increases the amount and/or activity of one or more of miR-193a/b, miR-365, miR-328, miR-378, miR-30b/c, miR-455, and miR-32, optionally as compared to expression and/or activity as compared to the absence of a perturbagen.

Embodiment 143: The method of any one of Embodiments 1-142, wherein the method decreases the amount and/or activity of one or more of miR-27, miR-34a, miR-133, and miR-155, optionally as compared to the expression and/or activity in the absence of a perturbagen.

Embodiment 144: A perturbagen for use in the method of any one of Embodiments 1-143.

Embodiment 145: A method for promoting the formation of a beige adipocyte, or an immediate progenitor thereof, comprising: exposing a starting population of stem/progenitor cells comprising a non-lineage committed mesenchymal stem cell nor adipocyte stem cell to a perturbation having a perturbation signature that promotes the transition of the starting population of stem/progenitor cells into a beige adipocyte, wherein the perturbation signature comprises increased expression and/or activity of one or more of genes selected from the genes designated as an “up” gene in the gene directionality column of Table 1 and/or a decreased expression and/or activity in the non-lineage committed adipocyte stem cell or mesenchymal stem cell of one or more genes selected from the genes designated as an “down” gene in the gene directionality column of Table 1.

Embodiment 146: The method of Embodiment 145, wherein the perturbation signature comprises an increase in expression and/or activity of one or more genes in the progenitor cell of an activation of a network module designated in the network module column of Table 1.

Embodiment 147: The method of Embodiment 145 or Embodiment 146, wherein the activation of one or more genes of the network module designated in the network module column of Table 1 comprises modulating expression and/or activity of 2 or more genes within a network module.

Embodiment 148: The method of any one of Embodiments 145-147, wherein the perturbation signature comprises an increase in expression and/or activity in the progenitor cell of two or more genes designated as an “up” gene in the gene directionality column of Table 1.

Embodiment 149: The method of any one of Embodiments 145-148, wherein the perturbation signature comprises a decrease in expression and/or activity in the progenitor cell of two or more genes designated as a “down” gene in the gene directionality column of Table 1.

Embodiment 150: A method of increasing a quantity of beige adipocytes, or immediate progenitors thereof, comprising: exposing a starting population of stem/progenitor cells comprising a non-lineage committed adipocyte stem cell or mesenchymal stem cell to a pharmaceutical composition that promotes the formation of lineage specific progenitor population selected from adipoblasts, Myf5− progenitor cells, beige preadipocytes, and/or beige adipocytes, the pharmaceutical composition promoting the transition of a primitive stem/progenitor population into the lineage specific progenitor population that has the capacity to differentiate into beige preadipocytes, and/or beige adipocytes or immediate progenitors thereof, wherein the pharmaceutical composition comprises at least one perturbagen selected from Table 3, or a variant thereof.

Embodiment 151: A method for treating a disease or disorder characterized by an abnormal number of beige adipocytes, white adipocytes, beige preadipocytes, and/or white preadipocytes, comprising: (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

Embodiment 152: A method for treating a disease or disorder characterized by an increased number of white adipocytes, and/or white preadipocytes, comprising: (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

Embodiment 153: A method for treating a disease or disorder characterized by a decreased number of beige adipocytes, and/or beige preadipocytes, comprising: (a) administering to a patient in need thereof a therapeutically effective amount of at least one perturbagen selected from Table 3, or a variant thereof, wherein the at least one perturbagen is capable of changing a gene signature in a progenitor cell; or (b) administering to a patient in need thereof a cell, the cell having been contacted with at least one perturbagen selected from Table 3, or a variant thereof, wherein the at least one perturbagen is capable of changing a gene signature in a progenitor cell.

Embodiment 154: The method of any one of Embodiments 150-153, wherein the disease or disorder is obesity, morbid obesity, morbid obesity prior to surgery, obesity linked inflammation, obesity linked gallbladder disease, obesity induced sleep apnea, hyperlipidemia, dyslipidemia, hypercholesterolemia, atherogenic dyslipidemia, fatty liver disease (FLD), nonalcoholic fatty liver disease (NAFLD), Non-Alcoholic Steatohepatitis (NASH), Prader-Willi Syndrome, insulin resistance, glucose intolerance, pre-diabetes, increased fasting glucose, diabetes mellitus, type 2 diabetes mellitus, inflammation, vascular inflammation, hypertension, endothelial dysfunction, atherosclerosis, arteriosclerosis, coronary heart disease, peripheral artery disease, stroke, or microvascular disease, arterial remodelling, cardiovascular disease (CVD), atherosclerotic cardiovascular disease, metabolic syndrome or a combination of any two or more thereof. obesity, hyperlipidemia, NAFLD, Type II Diabetes, inflammation, hypertension, cardiovascular disease, a metabolic disorder, excess body weight, atheromatous disease, β-cell dysfunction, heart disease, hyperglycemia, impaired glucose tolerance, an inflammatory disorder, latent autoimmune diabetes (LADA), nephropathy, neuropathy, retinopathy, Syndrome X, and/or type 1 diabetes.

Embodiment 155: The method of Embodiment 154, wherein the metabolic disorder is not caused, not driven substantially by, or not driven principally by a disorder of protein aggregation.

Embodiment 156: The method of Embodiment 154, wherein the metabolic disorder is driven, at least in part, by protein aggregates.

Embodiment 157: The method of any one of Embodiments 150-156, wherein the method alleviates one or more symptoms selected from the group of abdominal obesity, a body mass index (BMI) of 30 or higher, a blood triglyceride level of 150 or higher, a blood HDL level of less than 40 or higher in men, a blood HDL level of 50 or higher in women, a blood pressure of 130/85 or higher, a fasting blood sugar of 110 or higher, hyperlipidemia, dyslipidemia, hypercholesterolemia, atherogenic dyslipidemia insulin resistance, glucose intolerance, pre-diabetes, increased fasting glucose, diabetes mellitus, and a combination of any two or more thereof.

Embodiment 158: A method for selecting a subject for method of any one of Embodiments 150-157, comprising: obtaining from a subject having the disease or disorder a sample of cells comprising a non-lineage committed adipocyte stem cell or mesenchymal stem cell; and contacting the sample of cells with least one perturbagen selected from Table 3 or a variant thereof, wherein the at least one perturbagen alters a gene signature in the sample of cells, the subject is selected as a patient.

Embodiment 159: A method for selecting a subject for method of any one of Embodiments 150-158, comprising: obtaining from a subject having the disease or disorder a sample of cells comprising a non-lineage committed adipocyte stem cell or mesenchymal stem cell; and contacting the sample of cells with at least one perturbagen capable of altering a gene signature in a non-lineage committed adipocyte stem cell or mesenchymal stem cell, wherein the at least one perturbagen increases in the sample of cells the expression and/or activity of one or more genes selected from the genes designated as an “up” gene in the gene directionality column of Table 1, and/or decreases in the sample of cells the expression and/or activity of one or more genes selected from the genes designated as an “down” gene in the gene directionality column of Table 1, the subject is selected as a patient.

Embodiment 160: A method for selecting a subject for method of any one of Embodiments 150-159, comprising: obtaining from a subject having the disease or disorder a sample of cells comprising a non-lineage committed adipocyte stem cell or mesenchymal stem cell; and contacting the sample of cells with least one perturbagen selected from Table 3 or a variant thereof, wherein the at least one perturbagen increases in the sample of cells the expression and/or activity of one or more genes selected from the genes designated as an “up” gene in the gene directionality column of Table 1, and/or decreases in the sample of cells the expression and/or activity of one or more genes selected from the genes designated as an “down” gene in the gene directionality column of Table 1, the subject is selected as a patient.

Embodiment 161: The method of any one of Embodiments 153-160, wherein the perturbation signature comprises an increase in expression and/or activity of one or more genes in the progenitor cell of an activation of a network module designated in the network module column of Table 1.

Embodiment 162: The method of any one of Embodiments 153-161, wherein the activation of one or more genes of the network module designated in the network module column of Table 1 comprises modulating expression and/or activity of 2 or more genes within a network module.

Embodiment 163: The method of Embodiment 161 or Embodiment 162, wherein the one or more genes are selected from MRPL12, MIF, BIRC5, CDK1, UBE2C, CISD1, TOMM70A, RRS1, TRAP1, DLD, GAPDH, CDK4, MRPS16, GALE, CAT, PFKL, CEBPA, SCP2, LPGAT1, NT5DC2, GNB5, LAP3, HSPA4, PPARG, HSPD1, NOLC1, SDHB, PGAM1, TIMM9, CCNB2, IFRD2, MPC2, STMN1, PARP1, UBE2A, GSTZ1, SCARB1, HADH, PEX11A, ETFB, HSD17B10, PXMP2, CIAPIN1, DNAJC15, FKBP4, SMARCA4, BZW2, VDAC1, ISOC1, CYCS, G3BP1, CD320, MYLK, EIF4EBP1, and/or PHGDH.

Embodiment 164: The method of any one of Embodiments 153-163, wherein the perturbation signature comprises an increase in expression and/or activity in the progenitor cell of two or more genes designated as an “up” gene in the gene directionality column of Table 1.

Embodiment 165: The method of any one of Embodiments 153-164, wherein the perturbation signature comprises a decrease in expression and/or activity in the progenitor cell of two or more genes designated as a “down” gene in the gene directionality column of Table 1.

Embodiment 166: The method of Embodiment 164 or Embodiment 165, wherein the one or more genes are selected from the genes designated as an “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, or 55 genes selected from the genes designated as an “down” gene in the gene directionality column of Table 1.

Embodiment 167: The method of Embodiment 166, wherein the one or more genes are selected from JUN, ID2, ZFP36, BAMBI, HERPUD1, NCK1, MYC, SQSTM1, NFKBIA, IER3, TIPARP, HES1, CYB561, HSD17B11, FYN, PLSCR1, CIRBP, FOS, NUCB2, S100A13, RSU1, ASAH1, TIMP2, COL1A1, MYL9, LOXL1, COL4A1, DNM1, MMP2, PTGS2, SSBP2, RTN2, VAT1, GAA, PROS1, B4GAT1, PNP, DRAP1, PRSS23, IGFBP3, TPM1, ILK, SCRN1, FHL2, KDM5B, GADD45B, EGFR, GRN, SERPINE1, TMEM50A, CLTB, GNAI2, FKBP14, CALU, STXBP1, HTRA1, UGDH, PLA2G4A, EXT1, CTSL, NENF, TGFBR2, EGR1, P4HA2, GNAS, APP, HSPA1A, ITGB5, EPB41L2, GSTM2, ICAM1, CEBPD, and/or CHIC2.

Embodiment 168: The method of any one of Embodiments 153-167, wherein the at least one perturbagen is selected from Table 3, or a variant thereof, comprises at least 2, at least 3, at least 4, or at least 5 perturbagens selected from Table 3, or variants thereof.

Embodiment 169: The method of any one of Embodiments 153-168, wherein the at least one perturbagen is selected from Table 3, or a variant thereof, comprises at least 2, at least 3, at least 4, or at least 5 perturbagens selected from Table 3, or variants thereof.

Embodiment 170: A method of identifying a candidate perturbation for promoting the transition of a starting population of progenitor cells comprising an adipocyte stem cell or a mesenchymal stem cell into beige adipocytes or immediate progenitors thereof, the method comprising: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into beige adipocytes or immediate progenitors thereof following exposure of the population of cells to the perturbation; and identifying the perturbation as a candidate perturbation for promoting the transition of a population of progenitor cells into beige adipocytes or immediate progenitors thereof based on the perturbation signature, wherein the perturbation signature is an increase in expression and/or activity in the progenitor cell of one or more genes selected from the genes designated as an “up” gene in the gene directionality column of Table 1, and/or a decrease in expression and/or activity in the progenitor cell of one or more genes selected from the genes designated as an “down” gene in the gene directionality column of Table 1.

Embodiment 171: The method of Embodiment 170, wherein the perturbation signature is an increase in expression and/or activity in the progenitor cell of an activation of a network module designated in the network module column of Table 1.

Embodiment 172: The method of Embodiment 170 or Embodiment 171, wherein the activation of one or more genes of the network module designated in the network module column of Table 1 comprises modulating expression and/or activity of 2 or more genes within a network module.

Embodiment 173: The method of any one of Embodiments 170-172, wherein the perturbation signature is an increase in expression and/or activity in the progenitor cell of two or more genes designated as an “up” gene in the gene directionality column of Table 1.

Embodiment 174: The method of any one of Embodiments 170-173, wherein the perturbation signature is a decrease in expression and/or activity in the progenitor cell of two or more genes designated as a “down” gene in the gene directionality column of Table 1.

Embodiment 175: A method for making a therapeutic agent for a disease or disorder selected from obesity, hyperlipidemia, NAFLD, Type II Diabetes, inflammation, hypertension, and/or cardiovascular disease, comprising: (a) identifying a candidate perturbation according to the method of Embodiment 170 and (b) formulating the candidate perturbation as a therapeutic agent for the treatment of the disease or disorder.

Embodiment 176: A method for directing a change in cell state of a progenitor cell, comprising: contacting a population of cells comprising a progenitor cell with at least one perturbagen capable of altering a gene signature in the progenitor cell, wherein altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1, wherein the progenitor cell is an adipocyte stem cell or a mesenchymal stem cell; and the change in cell state provides an increase in the number of one or more of adipoblasts, Myf5− progenitor cells, beige preadipocytes, and/or beige adipocytes.

Embodiment 177: The method of Embodiment 176, wherein the increase in the number of adipoblasts, Myf5− progenitor cells, beige preadipocytes, and/or beige adipocytes is relative to the number of adipoblasts, Myf5− progenitor cells, beige preadipocytes, and/or beige adipocytes obtained from a population of progenitor cells that are not contacted with the at least one perturbagen

Embodiment 178: The method of Embodiment 176, wherein the increase in the number of adipoblasts, Myf5− progenitor cells, beige preadipocytes, and/or beige adipocytes is relative to the number of adipoblasts, Myf5− progenitor cells, beige preadipocytes, and/or beige adipocytes obtained from a population of progenitor cells prior to contacting with the at least one perturbagen

Embodiment 179: The method of Embodiment 176, wherein the at least one perturbagen is selected from Table 3, or a variant thereof.

Embodiment 180: The method of Embodiment 179, wherein the at least one perturbagen comprises at least 2, at least 3, at least 4, or at least 5 perturbagens selected from Table 3, or variants thereof.

Embodiment 181: The method of Embodiment 176, the method provides an increase in expression and/or activity of uncoupling protein 1 (UCP1).

Embodiment 182: The method of Embodiment 176, wherein the change in cell state provides an increase in the number of beige adipocytes.

Embodiment 183: The method of Embodiment 176, wherein the one or more genes are selected from MRPL12, MIF, BIRC5, CDK1, UBE2C, CISD1, TOMM70A, RRS1, TRAP1, DLD, GAPDH, CDK4, MRPS16, GALE, CAT, PFKL, CEBPA, SCP2, LPGAT1, NT5DC2, GNB5, LAP3, HSPA4, PPARG, HSPD1, NOLC1, SDHB, PGAM1, TIMM9, CCNB2, IFRD2, MPC2, STMN1, PARP1, UBE2A, GSTZ1, SCARB1, HADH, PEX11A, ETFB, HSD17B10, PXMP2, CIAPIN1, DNAJC15, FKBP4, SMARCA4, BZW2, VDAC1, ISOC1, CYCS, G3BP1, CD320, MYLK, EIF4EBP1, and/or PHGDH.

Embodiment 184: The method of Embodiment 176, wherein the one or more genes are selected from JUN, ID2, ZFP36, BAMBI, HERPUD1, NCK1, MYC, SQSTM1, NFKBIA, IER3, TIPARP, HES1, CYB561, HSD17B11, FYN, PLSCR1, CIRBP, FOS, NUCB2, S100A13, RSU1, ASAH1, TIMP2, COL1A1, MYL9, LOXL1, COL4A1, DNM1, MMP2, PTGS2, SSBP2, RTN2, VAT1, GM, PROS1, B4GAT1, PNP, DRAP1, PRSS23, IGFBP3, TPM1, ILK, SCRN1, FHL2, KDM5B, GADD45B, EGFR, GRN, SERPINE1, TMEM50A, CLTB, GNAI2, FKBP14, CALU, STXBP1, HTRA1, UGDH, PLA2G4A, EXT1, CTSL, NENF, TGFBR2, EGR1, P4HA2, GNAS, APP, HSPA1A, ITGB5, EPB41L2, GSTM2, ICAM1, CEBPD, and/or CHIC2.

Methods of Culturing Cells In Vitro to Perform Single-Cell Analyses

In carrying out the techniques described herein for identifying the causes of cell fate, it is useful to generate datasets regarding cellular-component measurements obtained from single-cells. To generate these datasets, a population of cells of interest may be cultured in vitro. Alternately, these datasets may be generated, from single cells that have not been previously cultured; for example, cells used in single cell analyses may be obtained from dissociated primary tissue. A non-limiting example of such methods is provided in Example 1. This latter method of generating datasets is often desirable if one wants to capture information of the primary cell/organ as close to the in vivo setting as possible. However, for cells undergoing culturing, single-cell measurements of one or more cellular-components of interest may be performed at one or more time periods during the culturing to generate datasets.

In some embodiments, cellular-components of interest include nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc. proteins), lipids, carbohydrates, nucleotides (e.g., adenosine triphosphate (ATP), adenosine diphosphate (ADP) and adenosine monophosphate (AMP)) including cyclic nucleotides such as cyclic adenosine monophosphate (cAMP) and cyclic guanosine monophosphate (cGMP), other small molecule cellular-components such as oxidized and reduced forms of nicotinamide adenine dinucleotide (NADP/NADPH), and any combinations thereof. In some embodiments, the cellular-component measurements comprise gene expression measurements, such as RNA levels.

Any one of a number of single-cell cellular-component expression measurement techniques may be used to collect the datasets. Examples include, but are not limited to single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, and so on. The cellular-component expression measurement can be selected based on the desired cellular-component to be measured. For instance, scRNA-seq, scTag-seq, and miRNA-seq measure RNA expression. Specifically, scRNA-seq measures expression of RNA transcripts, scTag-seq allows detection of rare mRNA species, and miRNA-seq measures expression of micro-RNAs. CyTOF/SCoP and E-MS/Abseq measure protein expression in the cell. CITE-seq simultaneously measures both gene expression and protein expression in the cell. And scATAC-seq measures chromatin conformation in the cell. Table 2 below provides links to example protocols for performing each of the single-cell cellular-component expression measurement techniques described herein.

TABLE 2
Example Measurement Protocols

| Technique | Protocol |
|---|---|
| RNA-seq | Olsen and Baryawno “Introduction to Single-Cell RNA Sequencing” |
| | Current Protocols in Molecular Biology. Volume122, Issue1, April 2018, |
| | e57 |
| Tag-seq | Rozenberg et al., “Digital gene expression analysis with sample |
| | multiplexing and PCR duplicate detection: A straightforward protocol”, |
| | BioTechniques, vol. 61, No. 1, March 2018 |
| ATAC-seq | Buenrostro et al., “ATAC-seq: A Method for Assaying Chromatin |
| | Accessibility Genome-Wide”, Curr Protoc Mol Biol. 2015; 109: 21.29.1- |
| | 21.29.9 |
| miRNA-seq | Faridani et al., “Single-cell sequencing of the small-RNA transcriptome” |
| | Nature Biotechnology volume 34, pages1264-1266 (2016) |
| CyTOF/SCoPE- | Bandura et al., “Mass Cytometry: Technique for Real Time Single Cell |
| MS/Abseq | Multitarget Immunoassay Based on Inductively Coupled Plasma Time-of- |
| | Flight Mass Spectrometry”, Anal Chem. 2009 Aug. 15; 81(16): 6813-22 |
| | Shahi et al., “Abseq: Ultrahigh-throughput single cell protein profiling with |
| | droplet microfluidic barcoding”, Scientific Reports volume 7, |
| | Article number: 44447 (2017) |
| | Budnik et al., “SCoPE-MS: mass spectrometry of single mammalian cells |
| | quantifies proteome heterogeneity during cell differentiation”, Genome |
| | Biology 2018 19: 161 |
| CITE-seq | Stoeckius et al., “Simultaneous epitope and transcriptome measurement |
| | in single cells”, Nature Methods, vol 14, pages 865-868 (2017) |

The cellular-component expression measurement technique used may result in cell death. Alternatively, cellular-components may be measured by extracting out of the live cell, for example by extracting cell cytoplasm without killing the cell. Techniques of this variety allow the same cell to be measured at multiple different points in time.

If the cell population is heterogeneous such that multiple different cell types that originate from a same “progenitor” cell are present in the population, then single-cell cellular-component expression measurements can be performed at a single time point or at relatively few time points as the cells grow in culture. As a result of the heterogeneity of the cell population, the collected datasets will represent cells of various types along a trajectory of transition.

If the cell population is substantially homogeneous such that only a single or relatively few cell types, mostly the “progenitor” cell of interest, are present in the population, then single-cell cellular-component expression measurements can be performed multiple times over a period of time as the cells transition.

A separate single-cell cellular-component expression dataset is generated for each cell, and where applicable at each of the time periods. The collection of single-cell cellular-component expression measurements from a population of cells at multiple different points in time can collectively be interpreted as a “pseudo-time” representation of cell expression over time for the cell types originating from the same “progenitor” cell. The term pseudo-time is used in two respects, first, in that cell state transition is not necessarily the same from cell to cell, and thus the population of cell provides a distribution of what transition processes a cell of that “progenitor” type is likely to go through over time, and second, that the cellular-component expression measurements of those multiple cell's expressions at multiple time points simulates the possible transition behavior over time, even if cellular-component expression measurements of distinct cells give rise to the datasets. As a deliberately simple example, even if cell X gave a dataset for time point A and cell Y gave a dataset for time point B, together these two datasets represent the pseudo-time of transition between time point A and time point B.

For convenience of description, two such datasets captured for a “same” cell at two different time periods (assuming a technique is used that does not kill the cell) are herein referred to as different “cells” (and corresponding different datasets) because in practice such cells will often be slightly or significantly transitioned from each other, in some cases having an entirely distinct cell type as determined from the relative quantities of various cellular-components. Viewed from this context, these two measurements of a single-cell at different time points can be interpreted as different cells for the purpose of analysis because the cell itself has changed.

Note that the separation of datasets by cell/time period described herein is for clarity of description, in practice, these datasets may be stored in computer memory and logically operated on as one or more aggregate dataset/s (e.g., by cell for all time periods, for all cells and time periods at once).

In some instances, it is useful to collect datasets where a “progenitor” cell of interest has been perturbed from its base line state. There are a number of possible reasons to do this, for example, to knock out one or more cellular-components, to evaluate the difference between healthy and diseased cell states. In these instances, a process may also include steps for introducing the desired modifications to the cells. For example, one or more perturbations may be introduced to the cells, tailored viruses designed to knock out one or more cellular-components may be introduced, clustered regularly interspaced short palindromic repeats (CRISPR) may be used to edit cellular-components, and so on. Examples of techniques that could be used include, but are not limited to, RNA interference (RNAi), Transcription activator-like effector nuclease (TALEN) or Zinc Finger Nuclease (ZFN).

Depending upon how the perturbation is applied, not all cells will be perturbed in the same way. For example, if a virus is introduced to knockout a particular gene, that virus may not affect all cells in the population. More generally, this property can be used advantageously to evaluate the effect of many different perturbations with respect to a single population. For example, a large number of tailored viruses may be introduced, each of which performs a different perturbation such as causing a different gene to be knocked out. The viruses will variously infect some subset of the various cells, knocking out the gene of interest. Single-cell sequencing or another technique can then be used to identify which viruses affected which cells. The resulting differing single-cell sequencing datasets can then be evaluated to identify the effect of gene knockout on gene expression in accordance with the methods described elsewhere in this description.

Other types of multi-perturbation cell modifications can be performed similarly, such as the introduction of multiple different perturbations, barcoding CRISPR, etc. Further, more than one type perturbation may be introduced into a population of cells to be analyzed. For example, cells may be affected differently (e.g., different viruses introduced), and different perturbations may be introduced into different sub-populations of cells.

Additionally, different subsets of the population of cells may be perturbed in different ways beyond simply mixing many perturbations and post-hoc evaluating which cells were affected by which perturbations. For example, if the population of cells is physically divided into different wells of a multi-well plate, then different perturbations may be applied to each well. Other ways of accomplishing different perturbations for different cells are also possible.

Below, methods are exemplified using single-cell gene expression measurements. It is to be understood that this is by way of illustration and not limitation, as the present disclosure encompasses analogous methods using measurements of other cellular-components obtained from single-cells. It is to be further understood that the present disclosure encompasses methods using measurements obtained directly from experimental work carried out by an individual or organization practicing the methods described in this disclosure, as well as methods using measurements obtained indirectly, e.g., from reports of results of experimental work carried out by others and made available through any means or mechanism, including data reported in third-party publications, databases, assays carried out by contractors, or other sources of suitable input data useful for practicing the disclosed methods.

As discussed herein, gene expression in a cell can be measured by sequencing the cell and then counting the quantity of each gene transcript identified during the sequencing. In some embodiments, the gene transcripts sequenced and quantified may comprise RNA, for example mRNA. In alternative embodiments, the gene transcripts sequenced and quantified may comprise a downstream product of mRNA, for example a protein such as a transcription factor. In general, as used herein, the term “gene transcript” may be used to denote any downstream product of gene transcription or translation, including post-translational modification, and “gene expression” may be used to refer generally to any measure of gene transcripts.

Although the remainder of this description focuses on the analysis of gene transcripts and gene expression, all of the techniques described herein are equally applicable to any technique that obtains data on a single-cell basis regarding those cells. Examples include single-cell proteomics (protein expression), chromatin conformation (chromatin status), methylation, or other quantifiable epigenetic effects.

The following description provides an example general description for culturing a population of cells in vitro in order to carry out single-cell cellular-component expression measurement multiple time periods. Methods for culturing cells in vitro are known in the art. Those of skill in the art will also appreciate how this process could be modified for longer/shorter periods, for additional/fewer single-cell measurement steps, and so on.

In one embodiment, the process for culturing cells in a first cell state into cells in a second cell state includes one or more of the following steps:

Day 0: Thaw cells in the first cell state into a plate in a media suitable for growth of the cells.

Day 1: Seed cells in the first cell state into a multi-well plate. If applicable, perform additional steps to affect gene expression by cells. For example, simultaneously infect with one or more viruses to activate or knock out genes of interest.

Perform gene expression measurement iteration ti for cells in the wells.

Day 1+l: Change media as needed if any additional processes are performed.

If applicable, perform gene expression measurement iteration tₗ for cells in the wells.

Day 1+m: Change media to media appropriate to support growth of cells in the second cell state.

If applicable, perform gene expression measurement iteration tₘ for cells in the wells.

Days 1+n, o, p, etc.: Media change as needed to support further cell state transition from the first cell state to the second cell state. If applicable, perform additional steps to affect further transition from the first cell state to the second cell state. For example, add perturbations of interest to push cells towards the second cell state.

If applicable, perform gene expression measurement iterations tₙ, tₒ, tₚ, etc., for cells in the wells.

Day q: Perform gene expression measurement iteration t_{q} for cells in the wells and in the second state.

Collect cells into a tube and stain in suspension with antibodies matched to genes/proteins of interest, thereby sorting/identifying cells without having to lyse/destroy them. This step also can identify surface proteins that might not be seen with as much resolution in the setting of the cytoplasm. Image with a cell imaging system such as the BD Celestra flow cytometer or similar instrument by acquiring the cells from each well or tube. Quantify of number of cells per well that are in the first cell state and the number of cells per well that are in the second cell state. These steps can be used with unfixed cells.

Any aspect or embodiment disclosed herein can be combined with any other aspect or embodiment as disclosed herein.

Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the disclosure. Certain terms are discussed herein to provide additional guidance to the practitioner in describing the compositions, the devices, the methods and the like of aspects of the disclosure and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

The term “perturbation” in reference to a cell (e.g., a perturbation of a cell or a cellular perturbation) refers to any treatment of the cell with one or more active agents capable of causing a change in the cell's lineage or cell state (or in the lineage or cell state of the cell's progeny). These active agents can be referred to as “perturbagens.” In embodiments, the perturbagen can comprise, e.g., a small molecule, a biologic, a protein, a protein combined with a small molecule, an antibody-drug conjugate (ADC), a nucleic acid, such as an siRNA or interfering RNA, a cDNA over-expressing wild-type and/or mutant shRNA, a cDNA over-expressing wild-type and/or mutant guide RNA (e.g., Cas9 system, Cas9-gRNA complex, or other gene editing system), or any combination of any of the foregoing. As used herein, a perturbagen classified as a “compound” may be a small molecule or a biologic. Also, a perturbagen classified as “overexpression of gene” may be cDNA over-expressing a wild-type gene or an mRNA encoding a wild-type gene. In embodiments, an mRNA may comprise a modified nucleotide that promotes stability of the mRNA and/or reduces toxicity to a subject. Examples of modified nucleotides useful in the present technology include pseudouridine and 5-methylcytidine. Where a perturbagen is (or includes) a nucleic acid or protein described by reference to a particular sequence, it should be understood that variants with similar function and nucleic acid or amino acid identity are encompassed as well, e.g., variants with about: 1%, 2%, 3%, 4%, 5%, 6%, 7%, 8%, 9%, 10%, 11%, 12%, 13%, 14%, 15%, or more, variation, i.e., having about: 99%, 98%, 97%, 96%, 95%, 94%, 93%, 92%, 91%, 90%, 89%, 88%, 87%, 86%, or 85% identity to the reference sequence; e.g., in some embodiments, having, for example, at least: 1, 2, 3, 4, 5, 6, 7, 8, 9, 10, 11, 12, 13, 14, 15, 20, 25, 30, 35, 40, 45, 50, or more, substitutions.

The term “progenitor” in reference to a cell (e.g., a progenitor cell) refers to any cell that is capable of transitioning from one cell state to at least one other cell state. Thus, a progenitor can differentiate into one or more cell types and/or can expand into one or more types of cell populations. In some embodiments, the progenitor cell is an adipocyte stem cell or a mesenchymal stem cell. In some embodiments, the progenitor cell is a cell selected from the group of mesenchymal stem cell, adipoblast, Myf5− progenitor cell, white preadipocyte, white preadipocyte, and beige preadipocyte.

As used herein, the terms “cell fate” and “cell state” are interchangeable and synonymous.

As used herein, the “administration” of an agent to a subject includes any route of introducing or delivering to a subject a compound to perform its intended function. Administration can be carried out by any suitable route, including orally, or parenterally (e.g. intravenously, intramuscularly, intraperitoneally, or subcutaneously). Administration includes self-administration and the administration by another.

The term “subject,” refers to an individual organism such as a human or an animal. In embodiments, the subject is a mammal (e.g., a human, a non-human primate, or a non-human mammal), a vertebrate, a laboratory animal, a domesticated animal, an agricultural animal, or a companion animal. In embodiments, the subject is a human (e.g., a human patient). In embodiments, the subject is a rodent, a mouse, a rat, a hamster, a rabbit, a dog, a cat, a cow, a goat, a sheep, or a pig.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”. Likewise, the term “and/or” covers both “or” and “and”.

Unless specifically stated or obvious from context, as used herein, the term “about” is understood as within a range of normal tolerance in the art, for example within 2 standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The disclosure will be further described in the following examples, which do not limit the scope of the disclosure described in the claims.

### EXAMPLES

The present technology is further illustrated by the following examples, which should not be construed as limiting in any way. For each of the examples below, any perturbagen described herein could be used. By way of example, but not by limitation, the perturbagen used in the examples below could be any one or more of the perturbagens listed in Table 3.

### Example 1: Single Cell Manifold Labelled with Cell States in Mice

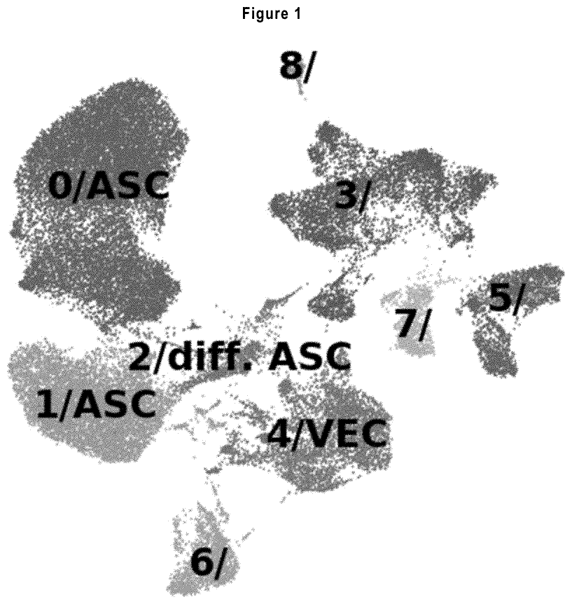

Mice were challenged with CL316243, a 33-receptor agonist, and the stromal vascular fraction (SVF) was purified from visceral (epididymal) and subcutaneous (inguinal) fat, sequenced at single cell level and compared to the gene expression of cells isolated from untreated mice. Based on this single-cell RNA sequencing (scRNA-seq) analysis, a subpopulation of cells, which were present only in SVF of visceral fat of mice treated with CL316243 and expressed 33-receptor and low levels of UCP1, a marker of beige fat, was identified. These analyses led to generation of predictions that drive the transition of cells from adipocyte stem cells to beige pre-adipocytes (FIG. 1). In this and the following examples, antibody directed against UCP1, a beige adipocyte marker, is listed below in Table 4.

TABLE 4
Antibodies for staining.

| Antibody | Fluorophore | Vendor | Clone | Cat # |
|---|---|---|---|---|
| Anti-UCP1 | N/A | Abcam | Rabbit Polyclonal | ab10983 |

These results indicate that increased expression of UCP1 is a marker of beige fat and that differentiate into beige-like adipocytes. Accordingly, detection of UCP1 using methods disclosed herein is useful for detecting the level of beige adipocytes in subjects suffering from obesity, hyperlipidemia, NAFLD, Type II Diabetes, inflammation, hypertension, and/or cardiovascular disease.

### Example 2: Test Compounds Predicted to Induce Beige State In Vitro Using 3T3L1 Cells

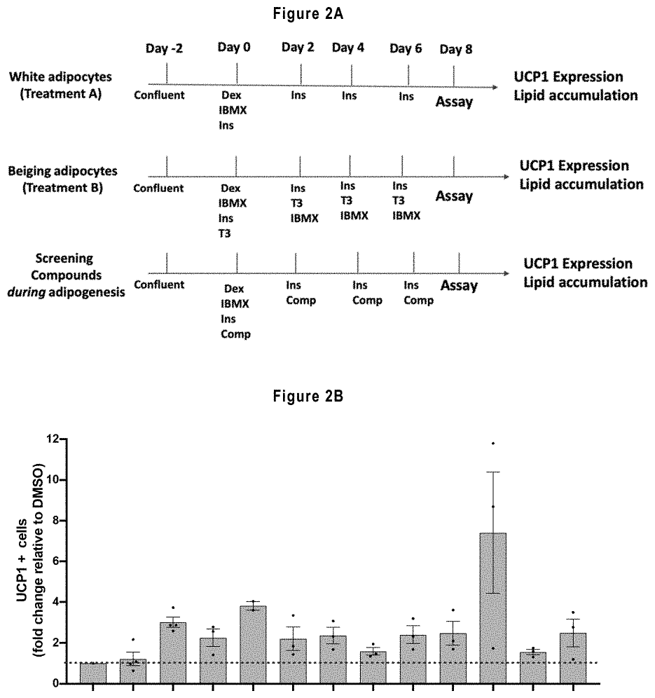

The pre-adipocytes 3T3L1 are fibroblast-like cells that derive from the mouse 3T3 fibroblasts. They have the ability to differentiate into white adipocytes, when they are stimulated with insulin, dexamethasone and IBMX (3-isobutyl-1-methylxanthine) for 2 days, followed by insulin for additional of 6-10 days. Following a modified protocol, the 3T3L1 pre-adipocytes can differentiate into beige-like adipocytes, as evidenced by the expression of UCP1. Upon confluency, they were treated with insulin, dexamethasone, IBMX and Triiodothyronine (T₃) for 2 days, followed by insulin, IBMX, and T₃ for 6-10 additional days. T3/IBMX was used as positive control and 37 compounds in this assay were tested (FIG. 2A). The readout was UCP1 staining by immunofluorescence, and the percentage of positively stained over total cells was assessed by high-content imaging. Thirty-seven compounds were tested along with the two concentrations tested. Table 5 shows the concentrations used for Perturbagens 1-12.

TABLE 5
Compounds tested in 3T3L1 cells

| Compound | Concentrations used |
|---|---|
| Perturbagen 12 | 1 μM and 10 μM |
| Perturbagen 1 | 0.1 μM and 1 μM |
| Perturbagen 10 | 0.1 μM and 1 μM |
| Perturbagen 2 | 1 μM and 10 μM |
| Perturbagen 3 | 0.1 μM and 1 μM |
| Perturbagen 9 | 0.1 μM and 1 μM |
| Perturbagen 4 | 0.1 μM and 1 μM |
| Perturbagen 11 | 1 μM and 10 μM |
| Perturbagen 8 | 0.1 μM and 1 μM |
| Perturbagen 5 | 1 μM and 10 μM |
| Perturbagen 7 | 0.1 μM and 1 μM |
| Perturbagen 6 | 1 μM and 10 μM |

As shown in FIG. 2B, of the thirty-seven compounds that were tested in two concentrations, five increased the number of UCP1 positive cells compared to DMSO-treated cells in three independent experiments. These compounds were Perturbagen 1, Perturbagen 2, Perturbagen 3, Perturbagen 4 and Perturbagen 5.

These results indicate that Perturbagen 1, Perturbagen 2, Perturbagen 3, Perturbagen 4 and Perturbagen 5 are useful to differentiate pre-adipocytes can differentiate into beige-like adipocytes. Accordingly, Perturbagen 1, Perturbagen 2, Perturbagen 3, Perturbagen 4 and Perturbagen 5 and/or variants thereof are useful in the methods of treatment of obesity, hyperlipidemia, NAFLD, Type II Diabetes, inflammation, hypertension, and/or cardiovascular disease in subjects in need thereof.

### Example 3: Testing Compounds In Vivo

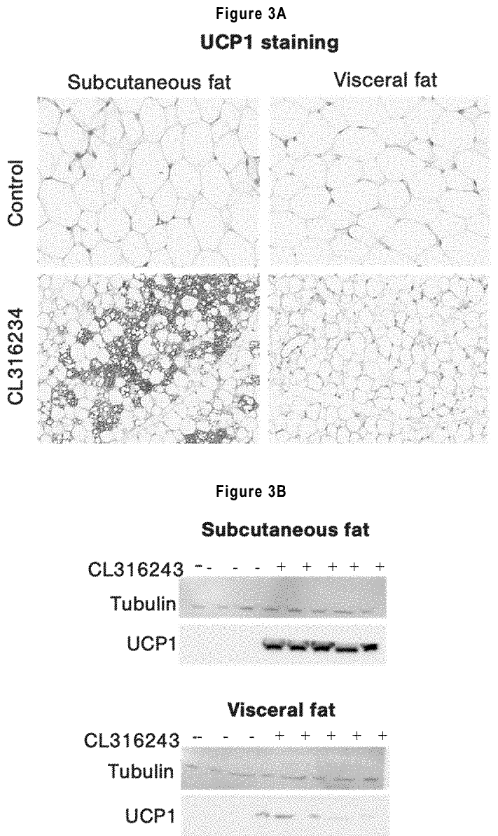

An in vivo assay of induction of beige adipocytes in visceral and subcutaneous fat depots was established using the β3-receptor agonist CL316243. 8-week old male C57Bl6/J mice were dosed with 1 mg/kg CL316243 with intraperitoneal injections, either every day for 1 week or every other day for two weeks. The vehicle was used as a negative control. Following the dosage administration, the mice were sacrificed, and epididymal (visceral) and inguinal (subcutaneous) fat was excised. The expression of UCP1 was assessed in the excised epididymal (visceral) and inguinal (subcutaneous) fat, by western blot and immunohistochemistry. As shown in FIG. 3A, CL 316234 treated mice showed increased expression of UCP1, as indicated by immunohistochemistry, in epididymal (visceral) as well as inguinal (subcutaneous) fat compared to control mice. The adipose tissue cells in epididymal (visceral) and inguinal (subcutaneous) fat from CL 316234 treated mice were also smaller compared to the cells from control mice (FIG. 3A). As shown in FIG. 3B, CL 316234 treated mice showed increased expression of UCP1, as shown by western blot analysis, in inguinal (subcutaneous, FIG. 3B (top panel)) epididymal (visceral, FIG. 3B (bottom panel)) as well as fat compared to the corresponding fat in control mice.

### Example 4: Single Cell Manifold Labelled with Cell States in Human Cells

Single cell analyses using human white or beige adipocytes will be performed to generate species-specific predictions. For these analyses, commercially available human visceral or subcutaneous pre-adipocytes isolated from lean, obese, or diabetic donors will be used and differentiated into white or beige adipocytes in the presence or absence of compounds known to induce beiging, including Triiodothyronine (T₃), Rosiglitazone, or BMP4 and BMP7. The compounds used in these studies will include CL316243, a β3-receptor agonist, as well as Perturbagen 1, Perturbagen 2, Perturbagen 3, Perturbagen 4 and Perturbagen 5 and/or variants thereof. Single cell analyses will be performed before, during and after differentiation into white or beige adipocytes, and single cell manifolds will be generated. The manifolds will be compared between the different adipose depots and treatments to generate hypothesis and predictions that will allow directing the differentiation of white pre-adipocytes into beige state efficiently.

### Example 5: Analysis of Mitochondrial and Beige Markers by Western Blot and Gene Expression Analysis

The pre-adipocytes 3T3L1 cells will be treated with Perturbagen 1, Perturbagen 2, Perturbagen 3, Perturbagen 4 and Perturbagen 5 and analogs thereof. DMSO will be used as a negative control as vehicle only control. Treated and untreated cells will be subjected to western blotting using antibodies against UCP1 and other mitochondrial and beige markers. The treated and untreated cells will also be subjected to gene expression analysis of UCP1 and other mitochondrial and beige markers.

These studies are expected to show that Perturbagen 1, Perturbagen 2, Perturbagen 3, Perturbagen 4 and Perturbagen 5 and/or variants thereof induce the UCP1 and other mitochondrial and beige markers compared to DMSO-treated cells, indicating that these compounds induce the differentiation of pre-adipocytes into beige-like adipocytes.

### Example 6: Test Compounds Predicted to Induce Beige State Using Human Preadipocytes

Commercially available human pre-adipocytes isolated from subcutaneous or omental adipose tissue from lean, obese, and diabetic donors will be obtained. The human pre-adipocytes will be differentiated into white adipocytes using commercially available media. To direct differentiation or induce trans-differentiation into beige adipocytes the same media supplemented with known inducers of beige cell state, including Triiodothyronine (T₃), Rosiglitazone, BMP4 and BMP7 as positive controls, or compounds predicted to induce the beige phenotypes (Examples 1-2) will be used. DMSO or vehicle only will be used as negative control. The expression of beige markers will be analyzed by Q-PCR, Western blot, and immunofluorescence. Functional analyses will be performed to determine if increased levels of UCP1 and other mitochondrial markers correlate with increased metabolic activity.

The results of these experiments are expected to show that Perturbagen 1, Perturbagen 2, Perturbagen 3, Perturbagen 4 and Perturbagen 5 and/or variants thereof induce the expression of expression of beige markers compared to control cells, indicating that these compounds induce the differentiation of pre-adipocytes into beige adipocytes. The results of these experiments are also expected to show that expression of UCP1 and other mitochondrial markers correlate with increased metabolic activity. The results of these experiments are also expected to show that the Perturbagen 1, Perturbagen 2, Perturbagen 3, Perturbagen 4 and Perturbagen 5 and/or variants thereof induce increased metabolic activity, increased levels of beige adipocytes in fat tissue. Accordingly, Perturbagen 1, Perturbagen 2, Perturbagen 3, Perturbagen 4 and Perturbagen 5 and/or variants thereof are useful in the methods of treatment of obesity, hyperlipidemia, NAFLD, Type II Diabetes, inflammation, hypertension, and/or cardiovascular disease in subjects in need thereof.

### Example 7: Testing Compounds In Vivo

Selected compounds predicted to induce the beige state (e.g. Perturbagen 1, Perturbagen 2, Perturbagen 3, Perturbagen 4 and Perturbagen 5 and/or variants thereof, See Example 1) will be tested in C57Bl6/J male mice fed chow or high fat diet (60% Kcal from fat). The treatments will be given either during high fat feeding intending to induce prophylactic effects or after the obese and diabetic disease states are established with the goal to reverse the disease. To test if the compounds can prevent obesity and diabetes, 6-8 week old mice (n=10-15) will be placed on high fat diet and dosed chronically with predicted compounds shown effective induction of beige state in vitro (Examples 2 and 3). The route of administration, the dose concentration and the frequency of doses will be determined by data available in the literature or by PK studies that will be performed. Body weights and food consumption will be monitored on a weekly basis. Insulin, leptin, glucose, fatty acids in the blood will be measured after O/N fasting and assess glucose handling by glucose tolerance and insulin tolerance tests after 8, 12 and 16 weeks on the regimen. When significant differences in metabolic parameters are observed, the animals will be sacrificed, the various fat depots will be weighed, their gene expression will be analyzed, inflammation will be assessed, and presence of beige adipocytes, and size of white adipocytes will be determined by histology.

After two weeks of the beginning of treatment a small number of animals (n=2-3) will be sacrificed to determine if beiging state in visceral and subcutaneous fat is induced before any physiological effects are noticeable. The level of induction of UCP1, which is a marker of beige adipocytes, will be assessed by western blotting, Q-PCR and immunohistochemistry.

Following a similar treatment scheme, animals that are obese and diabetic will be dosed, disease states induced by feeding high fat diet (60% kcal from fat) for 16 weeks. The animals will be dosed with compounds predicted or shown to induce beige states in vitro, at two different doses. The route of administration and the doses will be determined either by information available in the literature or by internally generated PK data. Body weight, food consumption will be assessed on a weekly basis, and metabolic parameters after 4, 8, and 12 weeks of compound treatment. If we significant differences are observed in any of these parameters, the animals will be sacrificed and their fat depots will be assessed by histology and gene expression analysis.

The results of these experiments are expected to show that Perturbagen 1, Perturbagen 2, Perturbagen 3, Perturbagen 4 and Perturbagen 5 and/or variants thereof induce the expression of expression of beige markers compared to control cells, indicating that these compounds induce the differentiation of pre-adipocytes into beige adipocytes in vivo. The results of these experiments are also expected to show that the Perturbagen 1, Perturbagen 2, Perturbagen 3, Perturbagen 4 and Perturbagen 5 and/or variants thereof reduce the severity of obesity, hyperlipidemia, NAFLD, Type II Diabetes, inflammation, hypertension, and/or cardiovascular disease in mouse models in vivo. Accordingly, Perturbagen 1, Perturbagen 2, Perturbagen 3, Perturbagen 4 and Perturbagen 5 and/or variants thereof are useful in the methods of treatment of obesity, hyperlipidemia, NAFLD, Type II Diabetes, inflammation, hypertension, and/or cardiovascular disease in subjects in need thereof.

### Example 8

### Pharmacokinetic Profile of Perturbagen 6

To determine the pharmacokinetic profile of compound Perturbagen 6 following single intravenous, intraperitoneal, subcutaneous, and oral gavage administration in male C57BL/6 mice.

### Rationale

Pharmacokinetics defines what the body does to Perturbagen 6 administered via various routes. Pharmacokinetics determines absorption, distribution, metabolism and elimination of a new chemical entity from the body. These pharmacokinetic properties determine the onset, intensity, and the duration of Perturbagen 6 in the body. First of all, the Perturbagen 6 absorption from the site of administration permits the entry of Perturbagen 6 to the plasma. Secondly, Perturbagen 6 then leaves plasma and distribute to the interstitial and intracellular fluids. Third, the drug is metabolized by the liver and other tissues, and finally, drug and its metabolites are eliminated from the body in urine, bile and feces. Thus, pharmacokinetics provides information of Perturbagen 6's absorption, distribution, metabolism and elimination from the body. In general terms, it can be explained as an effect of drug or chemical entity on body upon administration. In order to carryout in vivo studies for efficacy and proof of concept, it is important to determine the PK profile of Perturbagen 6 and its distribution. Therefore, exposure of Perturbagen 6 in the target tissues and overall pk parameters are important in choosing doses and designing the animal studies. In this study the pharmacokinetic profile of test compound Perturbagen 6 was done to select doses for in vivo efficacy studies.

Materials and Methods

Table 6 shows the details of the pharmacokinetic profile study of Perturbagen 6.

TABLE 6
Details of Pharmacokinetic Profile study of Perturbagen 6
Test Compound Details

| | |
|---|---|
| Compound | Test compounds |
| | VS-X4 |

Test System Details

| | |
|---|---|
| Gender/Strain/Species | Male C57BL/6 mice |
| Body weight range | 25-35 g |
| Age | 8-12 weeks |

Study Design

| | | | Dosing | | | |
|---|---|---|---|---|---|---|
| Test | | Dose | Volume | Strength | | Feeding |
| Compounds | Route | (mg/kg) | (mL/kg) | (mg/mL) | Vehicle | Regimen |
| Perturbagen 6 | IV | 0.25 | 5 | 0.25 | | Fed |
| | SC | 20 | 10 | 2 | | Fed |
| | PO | 20 | 10 | 2 | | Overnight |
| | | | | | | Fasted* |
| | IP | 10 | 10 | 2 | | Fed |

*All animals will be fasted over-night and feed will be provided 4 h post dose of test compound
Perturbagen 6 was formulated as follows:
PO: 1% Tween 80 + 0.5% Methyl cellulose q.s.
IV: 5% NMP, 5% Cremophor EL, 30% PG, 60% saline
SC: 10% NMP, 10% Cremophor EL, 20% PEG400, 10% PG, 50% saline
IP: 10% NMP, 10% Cremophor EL, 20% PEG400, 10% PG, 50% saline
pH of solution should be within 4-9 acceptable
Each group had 3 mice and the total number of groups was 4 as follows: PO, IV, SC, IP

| | |
|---|---|
| Sample collection and | 0.083 (IV only), 0.25, 0.5, 1, 2, 4, 6, 8, 24 h, post dose. |
| Storage | Approximately 25 μL of blood were collected at each time point through |
| | saphenous vein. All blood samples were centrifuged at 13000 rpm for 10 |
| | minutes at set temperature of 4° C. to collect plasma. The plasma samples |
| | were stored below −80° C. until shipment to the sponsor for bioanalysis. |
| Anticoagulant | 0.2% K₂ EDTA |
| Bioanalysis | All plasma samples were analyzed by discovery grade bioanalytical |
| | method developed for estimation of each test compound in plasma |
| | samples using LC-MS/MS systems. |
| Pharmacokinetic | Pharmacokinetic parameters of test compounds were calculated using |
| Analysis | the non-compartmental analysis tool of the WinNonlin 6.4 or higher |
| | version. Pharmacokinetic parameter viz. Cₒ, CL, terminal elimination half- |
| | life (t_{1/2}), Vₛₛ, Cₘₐₓ, Tₘₐₓ, AUCₗₐₛₜ, AUC_{inf}, and F (%) were estimated and |
| | reported. |

Annexure 1: Test Compound Details

| Compound ID. | Compound Name | Batch No. | Purity (%) | MW of Base | MW of Salt |
|---|---|---|---|---|---|
| Perturbagen 6 | Perturbagen 6 | NA | 98 | 523.97 | 523.97 |

1. Results

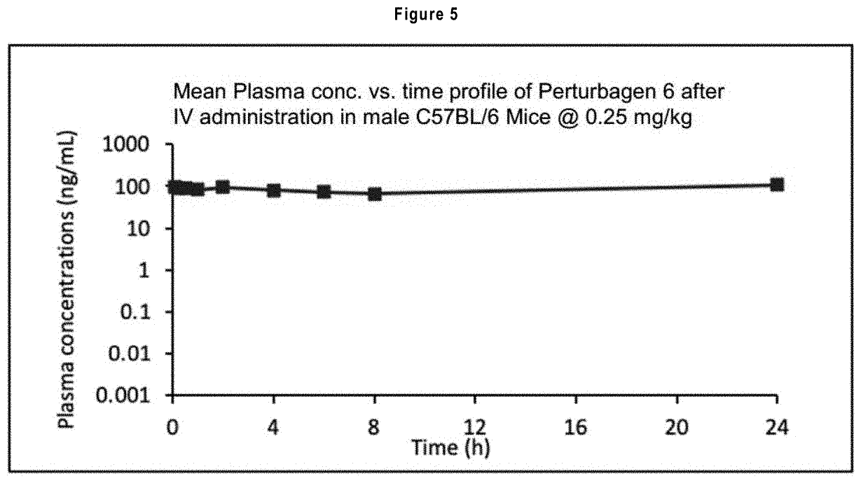

Plasma concentrations were measured at intervals shown in FIG. 5 following intravenous administration of the test agent Perturbagen 6 at a dose of 0.25 mg/kg body weight. Minimal changes were noticed in all 3 mice between 8 and 24 h post-dose. Several pk parameters were calculated from the data obtained from FIG. 5 and are shown in Table 7.

From the pharmacokinetic data in Table 7, it is not possible to calculate T_{1/2} and Cₘₐₓ because the curve remained flat and during the 24 h time following IV administration, an elimination phase was not evident, which made it difficult to calculate extrapolated AUC. However, the mean residence time could be calculated, which was around 13 h.

TABLE 7
Pharmacokinetic properties calculated from data obtained following
IV administration of Perturbagen 6 in male C57BL/6 mice.

| | Dose | | C₀ | T_{1/2} | Vss | Cl | AUCₗₐₛₜ | AUC_{inf} | AUCₑₓₜᵣₐ | MRTₗₐₛₜ |
|---|---|---|---|---|---|---|---|---|---|---|
| Compound ID | mg/kg | Mouse | (ng/mL) | (h) | (L/kg) | (ml/min/kg) | (h*ng/mL) | (h*ng/mL) | % | (h) |
| Perturbagen 6 | 0.25 | M0214 | 93.81 | NR¹ | NR | NR | 1451.14 | NR | NR | 12.35 |
| | | M0215 | 91.69 | NR | NR | NR | 1880.86 | NR | NR | 11.41 |
| | | M0216 | 110.79 | NR | NR | NR | 2825.23 | NR | NR | 15.07 |
| | | N | 3.00 | 0.00 | 0.00 | 0.00 | 3.00 | 0.00 | 0.00 | 3.00 |
| | | Mean | 98.76 | NA | NA | NA | 2052.41 | NA | NA | 12.94 |
| | | SD | 10.47 | NA | NA | NA | 702.92 | NA | NA | 1.90 |

¹NR, not reportable;
NA, not applicable

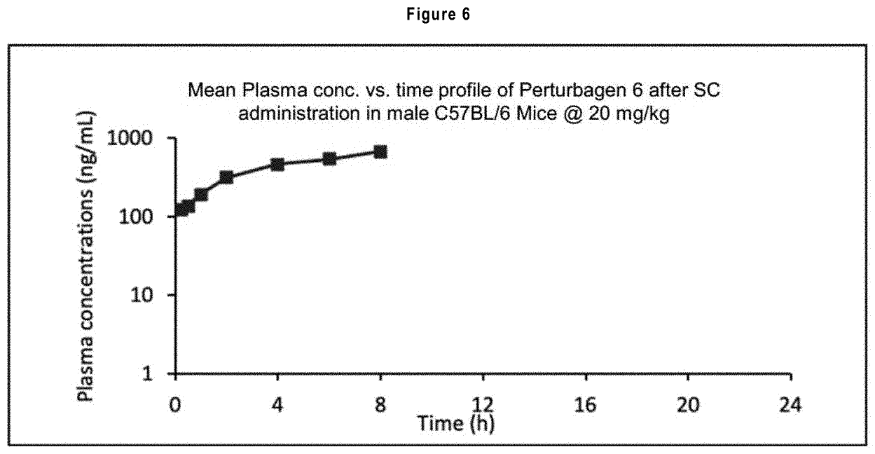

Next, the test agent, Perturbagen 6, was administered subcutaneously. Unlike IV administratation, absorption of the compound is not instantaneous, but gradual as evident from the shape of the curve (FIG. 6). The Cₘₐₓ in this study reached at Tₘₐₓ of 8 h, the last timepoint. The mean plasma AUC calculated at 8 h timepoint was 3357 h*ng/mL (Table 8). Mean bioavailability was found to be 2.04. It should be noted out of 3 animals in 24 timepoint one animal was found dead, Therefore, 24 h time point was not analyzed.

TABLE 8
Pharmacokinetic properties calculated from data obtained following
subcutaneous administration of Perturbagen 6 in male C57BL/6 mice.

| | Dose | | Cₘₐₓ | Tₘₐₓ | Tₗₐₛₜ | T_{1/2} | AUCₗₐₛₜ | AUC_{inf} | AUCₑₓₜᵣₐ | |
|---|---|---|---|---|---|---|---|---|---|---|
| Compound ID | mg/kg | Animal | (ng/mL) | (h) | (h) | (h) | (h*ng/mL) | (h*ng/mL) | % | % F |
| Perturbagen 6 | 20 | M0202 | 890.09 | 8.00 | 8.00 | ¹NR | 4236.69 | NR | NR | 2.58 |
| | | M0203 | 585.75 | 8.00 | 8.00 | NR | 2972.32 | NR | NR | 1.81 |
| | | M0204 | 525.36 | 8.00 | 8.00 | NR | 2863.90 | NR | NR | 1.74 |
| | | N | 3.00 | 3.00 | 3.00 | 0.00 | 3.00 | 0.00 | 0.00 | 3.00 |
| | | Mean | 667.07 | 8.00 | 8.00 | NA | 3357.64 | NA | NA | 2.04 |
| | | SD | 195.49 | 0.00 | 0.00 | NA | 763.21 | NA | NA | 0.46 |

¹NR, not reportable;
NA, not applicable

Administration of compounds by po route is the most common route. The plasma exposure curve suggested that absorption of the test agent took 2-3 h Perturbagen 6 (FIG. 7) since the mean Tₘₐₓ obtained from the 3 individual animals was 3 h (Table 4). While T_{1/2} could not be calculated from the data obtained, po administration of Perturbagen 6 showed good Cₘₐₓ (mean 258 ng·mL) and mean AUC at 24 h timepoint of 4111 h*ng/mL (Table 9). In po administration study too the plasma concentration remains pretty steady from 8 h timepoint to 24 h timepoint post-dosing. Better estimated AUC observed in po administration compared to SC administration. The po bioavailability was 2.50 (Table 9), somewhat better than the SC bioavailability (Table 8).

TABLE 9
Pharmacokinetic properties calculated from data obtained following
oral administration of Perturbagen 6 in male C57BL/6 mice.

| | Dose | | Cₘₐₓ | Tₘₐₓ | Tₗₐₛₜ | T_{1/2} | AUCₗₐₛₜ | AUC_{inf} | AUCₑₓₜᵣₐ | |
|---|---|---|---|---|---|---|---|---|---|---|
| Compound ID | mg/kg | Animal | (ng/mL) | (h) | (h) | (h) | (h*ng/mL) | (h*ng/mL) | % | % F |
| Perturbagen 6 | 20 | M0205 | 332.15 | 2.00 | 24.00 | NR | 5417.33 | NR | NR | 3.30 |
| | | M0206 | 268.21 | 1.00 | 24.00 | NR | 3607.01 | 9929.56 | 63.67 | 2.20 |
| | | M0207 | 174.26 | 6.00 | 24.00 | NR | 3308.97 | NR | NR | 2.02 |
| | | N | 3.00 | 3.00 | 3.00 | 0.00 | 3.00 | 1.00 | 1.00 | 3.00 |
| | | Mean | 258.21 | 3.00 | 24.00 | NA | 4111.10 | 9929.56 | 63.67 | 2.50 |
| | | SD | 79.42 | 2.65 | 0.00 | NA | 1141.00 | NA | NA | 0.69 |

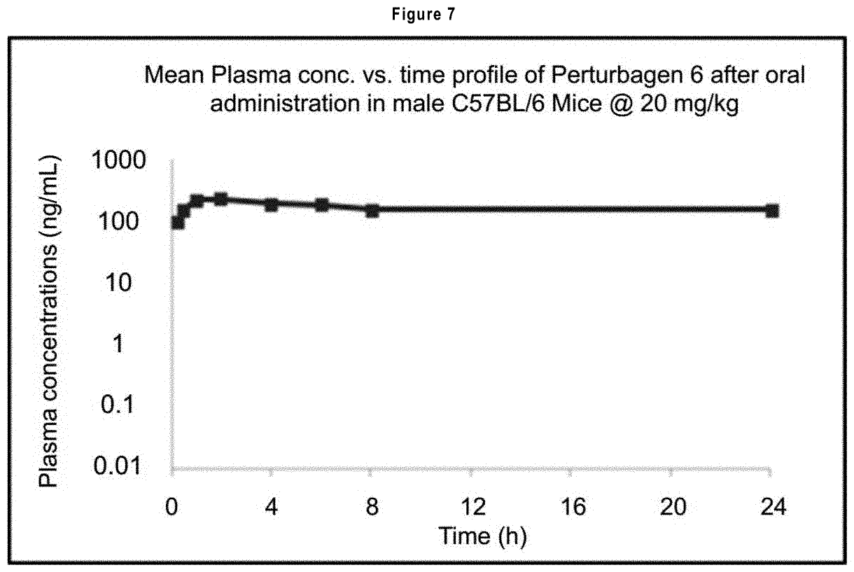

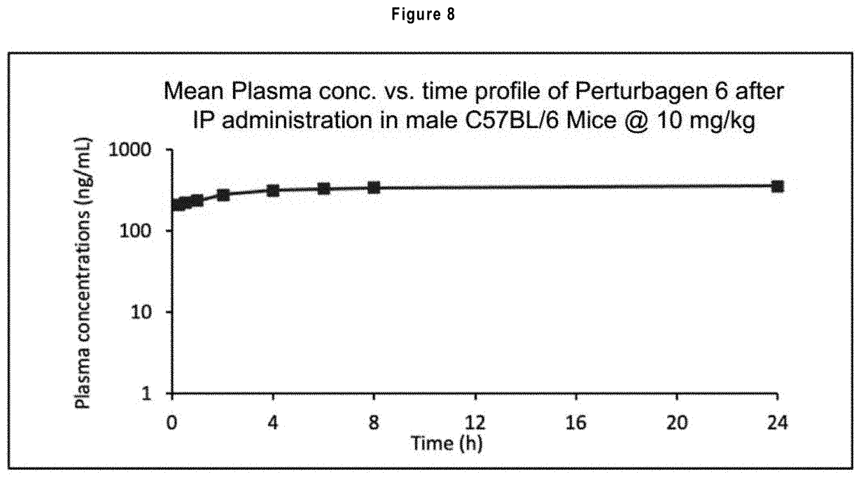

Pharmacokinetic properties of Perturbagen 6 was also evaluated in mice administered by the intraperitoneal route. The plasma exposure curve of test compound given by ip route appears very similar to the oral and SC administration, especially between 8 and 24 h timepoint post administration (FIG. 8). While T^{1/2} could not be estimated, ip route provided better exposure of the test agent compared to compound given by other routes. The estimated mean Cₘₐₓ and Tₘₐₓ were 363.74 ng/mL and 18.67 h, respectively. AUC at the 24 h timepoint was 7936 h*ng/mL and bioavailability 9.67. Overall pharmacokinetic properties were better when test compound was administered by ip route

TABLE 10
Pharmacokinetic properties calculated from data obtained following intraperitoneal
administration of Perturbagen 6 in male C57BL/6 mice.

| | Dose | | Cₘₐₓ | Tₘₐₓ | Tₗₐₛₜ | T_{1/2} | AUCₗₐₛₜ | AUC_{inf} | AUCₑₓₜᵣₐ | |
|---|---|---|---|---|---|---|---|---|---|---|
| Compound ID | mg/kg | Animal | (ng/mL) | (h) | (h) | (h) | (h*ng/mL) | (h*ng/mL) | % | % F |
| Perturbagen 6 | 10 | M0208 | 360.5 | 24 | 24 | NR | 7620.18 | NR | NR | 9.28 |
| | | M0209 | 393.86 | 8 | 24 | NR | 8636.46 | NR | NR | 10.52 |
| | | M0210 | 336.87 | 24 | 24 | NR | 7553.39 | NR | NR | 9.2 |
| | | N | 3 | 3 | 3 | 0 | 3 | 0 | 0 | 3 |
| | | Mean | 363.74 | 18.67 | 24 | NA | 7936.68 | NA | NA | 9.67 |
| | | SD | 28.63 | 9.24 | 0 | NA | 606.95 | NA | NA | 0.74 |

2. Non-Limiting Conclusions

From the pharmacokinetic analysis of the single dose administration of the test agent, following non-limiting conclusions were made:
The test compound, Perturbagen 6, showed useful bioavailability up to the last timepoint studied.
SC route showed best Cₘₐₓ.
IP route showed the best Tₘₐₓ and AUCₗₐₛₜ despite the dose being half (10 mg/kg) compared to sc and po routes (20 mg/kg).
The estimated AUCₗₐₛₜ was highest in the ip administered mice.
Because of the lack of test agent elimination phase, the T_{1/2} could not be estimated.

3. Summary

Perturbagen 6 was tested in a single dose study for its pharmacokinetic properties using four different routes of administration. Following single administration of Perturbagen 6, plasma samples were taken at various time points up to 24 time to measure compound concentration and plotted as concentration vs time points. Since an elimination phase was not evident from the plotted curves, the T_{1/2} could not be estimated. Mean estimated Tₘₐₓ for sc, po and ip routes administration were 8 h, 3 h, and 18.7 h, respectively. The mean estimated Cₘₐₓ for sc, po, ip, and iv (Co) routes were 667 ng/mL, 258.2 ng/mL, 363.7 ng/mL, and 98.7 ng/mL, respectively. Mean AUCₗₐₛₜ for iv, sc, po, and ip routes administration were 2052 h*ng/ml, 3357 h*ng/ml, 4111 h*ng/ml, and 7936 h*ng/ml, respectively. Mean bioavailability for sc, po, and ip routes administration were 2.04%, 2.50, and 9.67, respectively. These results suggest that the test agent Perturbagen 6 showed adequate exposure by all routes tested, however, ip route appears to be better than other routes.

### Example 9

### In-Vivo Study Efficacy for Perturbagen 6 in Diet-Induced Obese Mice (DIO-1) Study Objective

Using a prophylactic model of obesity, this study assessed if dosing of Perturbagen 6 in mice affected body weight gain, fat accumulation, and metabolic health.

The aim of the study was to assess if Perturbagen 6 improved diet-induced obesity and glucose homeostasis in mice fed a high fat diet. Fasting plasma glucose, insulin and leptin were measured. Adipose tissue depots (iWAT & eWAT) were collected and adipocyte size was analyzed by histology.

Materials and Methods

Experimental Design

Stratification/Randomization

200 male C57BL/6J mice were ordered from Jackson Laboratories (Jax #000664) at 4 weeks of age. The mice were group housed and were ˜8 weeks of age at study initiation. Prior to study initiation, mice were randomly assigned into 14 groups.

Study Details

Mice were housed on alpha-dri bedding (Innovive) in the Innovive caging system on a 12-hour light-dark cycle (0600-1800) at 68-79° F. and 30-70% humidity. Mice were allowed continued access to water and either D12492i (60% kcal fat) or D12450Bi (10% kcal fat) ad libitum. Animal handling and procedures were conducted under approved protocols and/or guidelines.

Mice were dosed by IP injection every other day (EOD) in the morning for 9 weeks. Body weights were recorded weekly. Food consumption was recorded weekly over three days (M-W). Dose volumes were adjusted by weight approximately every two weeks. The morning of day 15, 2 mice per group were euthanized via CO₂. On day 55, all groups were fasted overnight. On day 56, Groups were bled via the submandibular vein. Blood was collected on ice into EDTA-treated tubes. Blood glucose was measured using OneTouch Ultra 2 glucometers. Mice were euthanized via CO₂ on days 65 (Vehicle) and 66 (Perturbagen 6). Prior to euthanasia, all groups were fasted overnight.

Organ/Tissue/Blood Collection

Blood was collected on ice via cardiac stick into EDTA-treated tubes for ELISA analysis. iWAT & eWAT adipose depots as well as gastrocnemius skeletal muscle were dissected from each mouse. The left iWAT & eWAT adipose depots were fixed in 4% paraformaldehyde overnight at 4° C. The right depots were frozen for snap-frozen for follow-up studies. The following morning fixed tissues were washed 3× with PBS and stored in 70% EtOH at room temperature until histological analysis.

Treatment Groups

Table 11 shows the details of the treatment groups, including compound administered, dose, formulation, volume of administration, group size, and route of administration (ROA).

TABLE 11
Treatment groups.

| | | | | Volume | | |
|---|---|---|---|---|---|---|
| | Group | | | admin. | Group | |
| Group | treatment | Dose (mg/kg) | Formulation(mg/ml) | (ml/kg) | size | ROA |
| 1 | Vehicle | n/a | n/a | 10 | 14 | IP |
| 2 | Perturbagen 6 | 10 | 1 | 10 | 14 | IP |

Formulation

The vehicle formulation used in the present example was:

Vehicle: 80:20 5% dextrose:cremophor/kolliphor

Samples Analysis

Plasma Samples

Blood glucose was measured using OneTouch Ultra 2 glucometers.

Mouse Leptin ELISA kit (EMD Millipore, EZML-82K) and Rat/Mouse Insulin ELISA Kit (EMD Millipore, EZRMI-13K) were used to assay plasma samples. For each analyte, all the plasma samples obtained from the study were assayed on the same day. Prior to starting the experiment, the ELISA kits stored at 4 C were pre-warmed to room temperature (RT) and the plasma samples stored at −80° C. were thawed at 4° C. All incubations were performed at RT on a plate shaker rotating at 450 rpm, unless stated otherwise.

For the leptin ELISA, the microtiter plates were washed three times with 300 μL of 1× HRP Wash Buffer (made by diluting 10× HRP Wash Buffer Concentrate (EMD Millipore, EWB-HRP) 1:10 with distilled water) per well. 30 μL of Assay Buffer was added to background, standard, and Mouse Leptin Quality Control 1 and 2 (QC1 and QC2) wells, and 40 μL of Assay Buffer to sample wells. 10 μL of Matrix Solution was then added to the background, standard, and QC1 and QC2 wells. 10 μL of Assay Buffer was then added to background wells and 10 μL of Mouse Leptin Standards [range: 0.23 to 30 ng/mL] (EMD Millipore, E8082-K) was added to the standards wells in the order of ascending concentration. 10 μL of QC1 and QC2 (EMD Millipore, E6082-K) was added to the respective wells and 10 μL of samples to be assayed to the respective wells. 50 μL of Antiserum Solution (EMD Millipore, EAS83) was then added to each well and the plates were incubated for two hours.

The solutions were then decanted, and the wells washed three times with 300 μL of 1× HRP Wash Buffer. 100 μL of Detection Antibody (EMD Millipore, E1083) was added to each well and the plates were incubated for one hour. The solutions were then decanted, and the wells washed three times with 300 μL of 1× HRP Wash Buffer. 100 μL of Enzyme Solution was added to each well and the plates were incubated for 30 minutes. The solutions were then decanted, and the wells washed six times with 300 μL of 1× HRP Wash Buffer. 100 μL of Substrate Solution was added to each well and the plates were incubated until blue color was observed in the wells containing leptin standards, proportional to increasing leptin concentrations. It took 22 minutes for this color to be observed. 100 μL of Stop Solution (EMD Millipore, ET-TMB) was added to each well to stop the reaction. Absorbance values from the plates were read at 450 nm and 590 nm using a plate reader. Calculations and statistical analysis are described below.

For the insulin ELISA, the microtiter plates were washed three times with 300 μL of 1× HRP Wash Buffer (made by diluting 10× HRP Wash Buffer Concentrate 1:10 with distilled water) per well. 10 μL of Assay Buffer was added to blank and sample wells and 10 μL of Matrix Solution added to the blank, standard, and Quality Control 1 and 2 (QC1 and QC2) wells. 10 μL of Rat/Mouse Insulin Standards [range: 0.2 to 10 ng/mL] was added to the standards wells in the order of ascending concentration. 10 μL of QC1 and QC2, and 10 μL of samples to be assayed was added to the respective wells. 80 μL of Detection Antibody was then added to each well and the plates were incubated for two hours. The solutions were then decanted, and the wells washed three times with 300 μL of 1× HRP Wash Buffer. 100 μL of Enzyme Solution was added to each well and the plates were incubated for 30 minutes. The solutions were then decanted, and the wells washed six times with 300 μL of 1× HRP Wash Buffer. 100 μL of Substrate Solution was added to each well and the plates were incubated until blue color was observed in the wells containing insulin standards, proportional to increasing insulin concentrations. It took 20 minutes for this color to be observed. 100 μL of Stop Solution was added to each well to stop the reaction. Absorbance values from the plates were read at 450 nm and 590 nm using a plate reader. Calculations and statistical analysis are described below.

Tissues

Histology: Formalin-fixed soft tissue samples were dehydrated through graded alcohols, cleared with xylene, and infiltrated with paraffin wax. The tissues were then embedded in paraffin molds for sectioning on a microtome. Four-micron tissue sections were transferred to a water bath and picked up on charged glass microscope slides. Slides then had wax removed, rehydrated, and stained with general Hematoxylin & Eosin protocol. Finally, slides were coverslipped using permanent, toluene-based mounting media.

For immunohistochemical (IHC) staining in FFPE mouse tissue, staining was conducted on a Leica Bond RXm using standard chromogenic methods. For antigen retrieval, slides were heated in a pH9 EDTA based buffer for 2 hours at 70° C. followed by a 30 minute antibody incubation. The antibody used to detect UCP1 is the ERP20381 clone, ab209483 (abcam) at 1:200 dilution. Antibody binding was detected using an HRP-conjugated secondary polymer, followed by chromogenic visualization with diaminobenzidine (DAB). A Hematoxylin counterstain was used to visualize nuclei.

Image Analysis: Images captured on the Aperio Imaging System (HistTox Labs) were viewed using ObjectiveView™ (Objective Pathology) at a magnification of 0.8×. Two non-overlapping images were cropped for each section and exported as jpeg images (50% compression, no color adjustments) for further analysis in MetaXpress software (Molecular Devices).

To sample as many adipocytes as possible, multiple, non-overlapping regions of interest (ROI) were applied to each image. The default size for each ROI was 2500×2500 pixels but ROIs were modified in shape as necessary to maximally sample the adipocyte area without including blood vessels or damaged tissue; ROI sizes and locations were saved for each image of a section for future reference. Custom-written journals were used in MetaXpress to quantify adipocyte area and diameter sequentially within all ROI logging all metrics to a text file for further analysis.

Briefly, for each ROI, the image was duplicated at 100× magnification and then color thresholded using an emporically determined threshold to capture the internal area of each adipocyte. An inclusive color threshold was established for each slide and was set between 230-240 and 255 for each color channel. The thresholded image was binarised and an erode function for 4 neighboring pixels was iteratively run 10× to remove noise and more clearly define cell shapes. Morphometric analysis was then performed on the eroded image to detect objects with a size greater than 300 pixels and a shape factor greater than 0.2. For each object in the image, the calibrated area and diameter were logged. The log of the objects for the 2 images for each section were combined and a normalized frequency histogram generated in Microsoft Excel. To determine the fraction of the population that was small adipocytes, the area under the normalized frequency histogram below 1000pm² was determined.

Animal Information

Table 12 shows information of the animals used in the study.

TABLE 12
Animal Information.

| | Number of days | | | | |
|---|---|---|---|---|---|
| | from delivery | | | | |
| Strain | date to study date | Age | Vendor | Sex | Diet |
| C57BL/6J | 22 days | 4 | Jax | Male | D12492i (60% |
| | | weeks | | | kcal fat) |

Statistical Analysis

GraphPad Prism software, Version 8.4.2 was used for all graphing and statistical analysis. Normality will be tested via a D'Agostino-Pearson omnibus normality test or a Shapiro-Wilk normality test, and visual inspection of Log transformed data and residuals. If the samples are normally distributed, statistical significance will be determined using an unpaired two-tailed t-test or ordinary One-way ANOVA and Dunnett's ad hoc testing versus vehicle treated group will be performed. Data without a normal distribution were Log transformed and then analyzed using the above tests.

Results

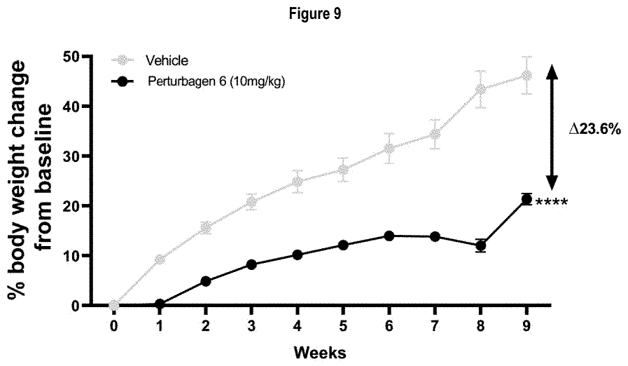

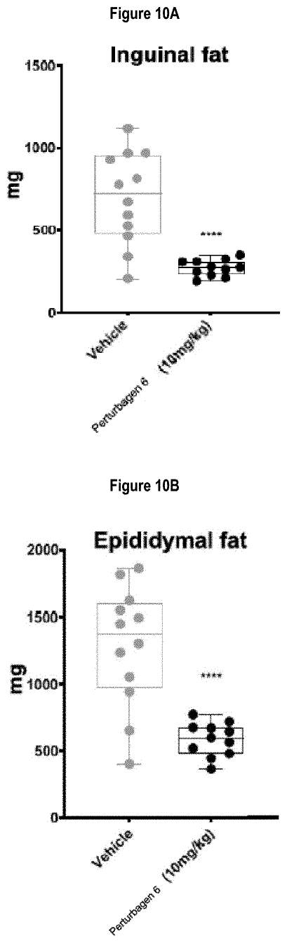

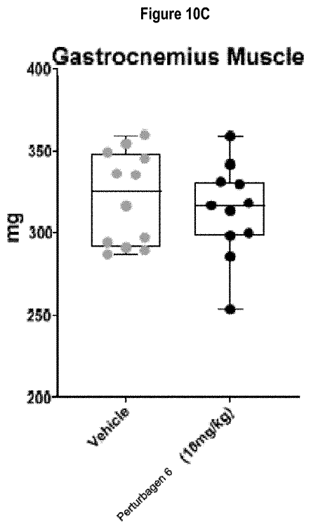

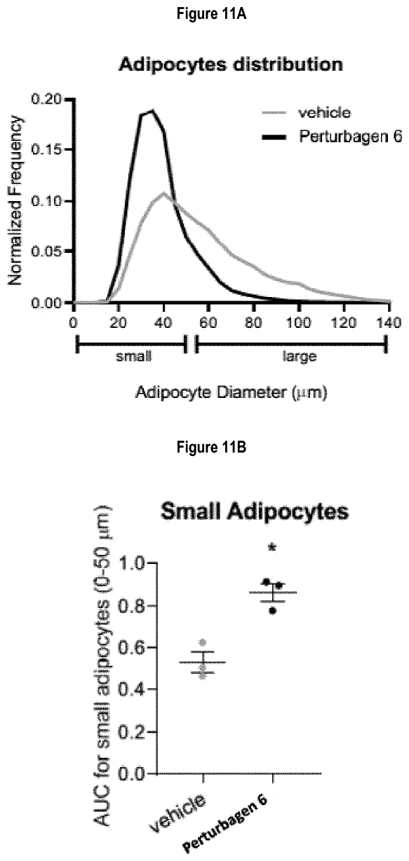

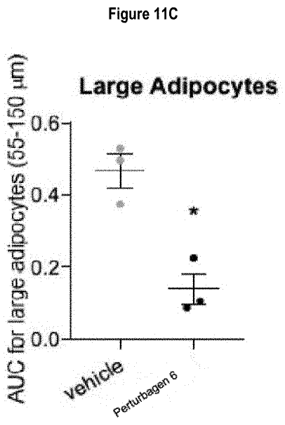

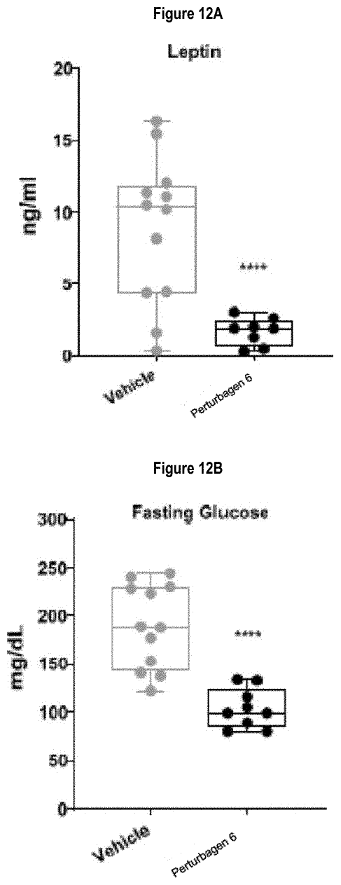

Perturbagen 6 treatment reduced weight gain by 23.6% compared to vehicle by week 9 of high fat diet feeding (FIG. 9). The reduction in bodyweight was concomitant with a decrease in visceral (epididymal) and subcutaneous (inguinal) fat mass but no reduction in gastrocnemius skeletal muscle (FIGS. 10A-10C). Histological analysis of the inguinal fat depot revealed that Perturbagen 6 altered the adipocyte size distribution by increasing the number of small adipocytes and reducing the number of large adipocytes (FIGS. 11A-11C). In agreement with the lower weight gain, mice treated with Perturbagen 6 also had reduced levels of plasma leptin, an adipokine correlating with fat mass (FIGS. 12A-12D). Additionally, mice treated with Perturbagen 6 had reduced fasting glucose, fasting insulin and reduced HOMA-IR, an index of insulin resistance (FIGS. 12A-12D). Without limitation, altogether, data suggest that treatment with Perturbagen 6 reduced weight gain and improved glycemic control in a mice model of diet induced obesity.

### Example 10

### In Vivo Study—Efficacy of Perturbagen 6 on DIO mouse model (DIO-2)

Study Objective

Previous studies demonstrated that Perturbagen 6 reduces weight gain when dosed to mice fed with high fat diet (60%), a phenotype that correlates with less fat accumulation and smaller adipocytes. This Example describes a follow-up efficacy study to assess the dose-dependent effect of Perturbagen 6 on body weight and body fat as well as metabolic health at lower orally administered daily doses in a mouse model of established obesity.

As described in this Example, this study tests the efficacy of four oral doses of Perturbagen 6. Study readouts include bodyweight, food consumption, glucose tolerance tests, insulin measurements, fat tissue weights and compound exposure in plasma and various tissues. Liraglutide was included as a standard of care control for weight loss.

Materials and Methods

Experimental Design

Stratification/Randomization

Sixty male C57BL/6J DIO mice were ordered from JAX (Jax #380050) at 24 weeks old, after being fed for 18 weeks on 60% kcal HFD (Research Diets #D12492). Upon arrival to the facility, mice were singly housed and allowed to acclimate for 5-7 days. Two extra mice were included to allow for better randomization and any transport issues. Prior to the start of the study, mice were randomized into 6 groups according to body weight.

Study Details

Weeks 1-6:

Mice were housed on corncob bedding (ScottPharma) in Innovive caging system on a 12-hour light-dark cycle (0700-1900) at 68-74° F. and 30-70% humidity. They were allowed continual to access water and high-fat diet (Research Diet D 12942) ad libitum. Animal handling and procedures were conducted under approved protocols and/or guidelines.

Mice were dosed by oral gavage or SC injection once daily, every day in the morning. Body weights were recorded daily and dosing solution was adjusted according to weight. Food consumption was measured overnight, 3 times per week from 7 p.m. to 8 a.m. (13 hours).

If bodyweight loss was observed in treated mice at the end of week 4, an oral Glucose Tolerance Test (GTT) in groups 1, (potentially 2, 3, 4) & 5 was performed on week 5.

Week 5: oGTT/Blood Collection for Glucose and Insulin

On the late afternoon on the day before the oGTT, the mice were placed in new cages at 7 p.m. One g of food was added inside the cage with full access to water. At 9 a.m. the following day, all mice were bled (by tail vein nick) for basal fasting conditions (t=0) for both glucose and insulin, followed immediately by oral gavage of 2 g/kg of a 400 mg/ml glucose solution diluted from D-(+)-Glucose solution 45% in H₂O, sterile-filtered, (Sigma, #G8769) with sterile water.

Blood glucose levels were measured in duplicate using a calibrated CareTouch glucometer at 0 (basal), 15, 30, 60 & 120 minutes after glucose challenge. In addition, collected ˜30 μl blood by tail vein into EDTA tubes was placed on ice following each glucose measurement. Blood was spun for plasma collection as soon as possible and plasma was stored at −80° C.

The daily test article dosing was given immediately after the oGTT is completed. Mice were returned to home cage with full access to feed and water.

Week 6: Euthanasia/Sample Collection

On the day before sacrifice, the mice were placed in new cages at 7 p.m. One g of food was added inside the cage with full access to water.

At 9 a.m. the following day, all mice were bled (by tail vein nick) for basal fasting conditions for both glucose and insulin.

Blood glucose levels (CareTouch glucometer) were measured in duplicate. ˜30 μl blood by tail vein was collected into EDTA tubes placed on ice following glucose measurement. Blood was spun for plasma collection as soon as possible and plasma was stored at −80° C.

Mice were euthanized via CO2 and bled via cardiac puncture into EDTA tubes on ice. Blood was spun (˜200 ml) for plasma collection (2 aliquots) and plasma stored at −80° C.

Treatment Groups

Table 13 shows the details of the treatment groups, including compound administered, dose, formulation, volume of administration, group size, and route of administration (ROA).

TABLE 13
Treatment groups.

| | | | | | Volume | | |
|---|---|---|---|---|---|---|---|
| | Group | | | | admin. | Group | |
| Group | treatment | Dose (mg/kg) | Formulation(mg/ml) | | (ml/kg) | size | ROA |
| 1 | Vehicle | n/a | N/A | | 5 | 10 | PO |
| 2 | Perturbagen 6 | Day 1 loading dose: 0.1, | 0.02 | mg/mL | 5 | 10 | PO |
| | | remaining days: 0.05 | 0.01 | mg/mL | | | |
| 3 | Perturbagen 6 | Day 1 loading dose: 0.5, | 0.1 | mg/mL | 5 | 10 | PO |
| | | remaining days: 0.25 | 0.05 | mg/mL | | | |
| 4 | Perturbagen 6 | Day 1 loading dose: 1, | 0.2 | mg/mL | 5 | 10 | PO |
| | | remaining days: 0.5 | 0.1 | mg/mL | | | |
| 5 | Perturbagen 6 | Day 1 loading dose: 10, | 2 | mg/mL | 5 | 10 | PO |
| | | remaining days: 5 | 1 | mg/mL | | | |
| 6 | Liraglutide | 0.4 | 0.08 | mg/mL | 5 | 8 | SC |

Formulation

The vehicle formulations used in the present example are as follows:

Vehicle for Perturbagen 6 (Groups 1-5): 1% Tween 80 & Methyl Cellulose

Vehicle for Liraglutide (Group 6): 50 mM phosphate buffer, propylene glycol (1.85% wt/vol), 0.04% Tween 80 (pH 7.5)

Animal Information

Table 14 shows information of the animals used in the study.

TABLE 14
Animal Information.

| | Number of days | | | | |
|---|---|---|---|---|---|
| | from delivery | | | | |
| Strain | date to study date | Age | Vendor | Sex | Diet |
| C57BL/ | 7 | 24 | Jax | Males | D12492i (60% |
| 6J DIO | | weeks | | | kcal fat) |

Statistical Analysis

GraphPad Prism software, Version 8.4.2 was used for all graphing and statistical analysis. Normality will be tested via a D'Agostino-Pearson omnibus normality test or a Shapiro-Wilk normality test, and visual inspection of Log transformed data and residuals. If the samples are normally distributed, statistical significance will be determined using an unpaired two-tailed t-test or ordinary One-way ANOVA and Dunnett's ad hoc testing versus vehicle treated group will be performed. Data without a normal distribution will be Log transformed and then analyzed using the above tests. Data without a normal distribution after log transformation will be analyzed using a non-parametric tests such as Mann-Whitney test (2 group comparisons) and Kruskal-Wallis test and Dunn's multiple comparison test.

Results

Body Weight

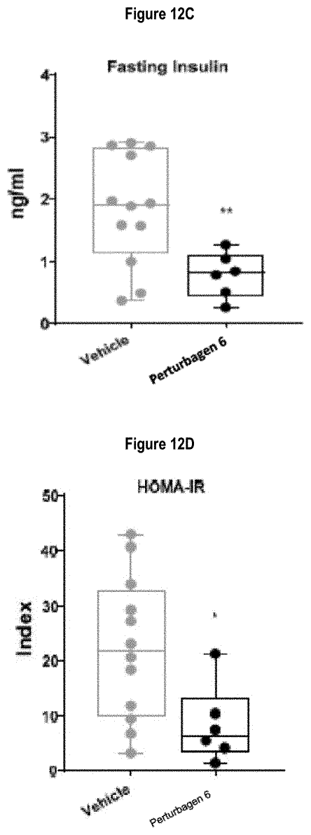

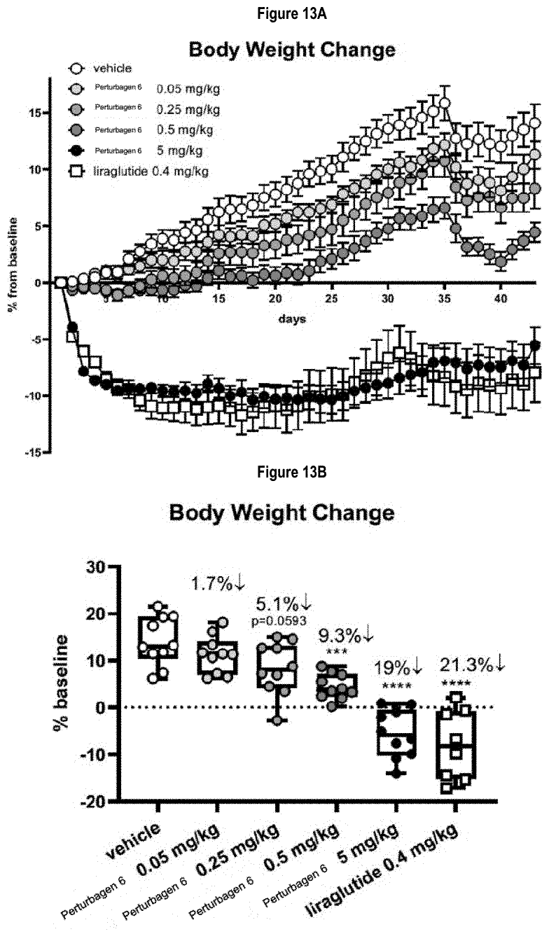

As shown in FIG. 13A, Perturbagen 6 doses ranging from 0.05 mg/kg to 0.5 mg/kg induced a dose-dependent reduction in body weight gain which was maintained throughout the whole experiment when compared to Vehicle group. Treatment with Perturbagen 6 at 5 mg/kg induced a weight loss comparable to Liraglutide throughout the experiment (FIG. 13A). Terminal body weight analysis shows that Perturbagen 6 doses ranging from 0.05 mg/kg to 0.5 mg/kg dose dependently reduced weight gain when compared to vehicle. When dosed at 5 mg/kg, Perturbagen 6 induced weight loss comparable to Liraglutide treated group (FIG. 13B). These data demonstrate that Perturbagen 6 dose dependently reduced weight gain in response to a high fat diet.

Food Intake

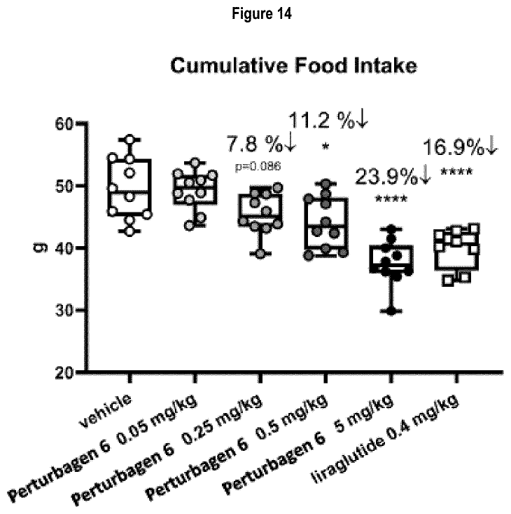

To assess if Perturbagen 6 had an effect on appetite, daily food intake was measured throughout the experiment and cumulative food intake was calculated for all groups. Analysis of cumulative food intake during the whole experiment showed that Perturbagen 6 treatment induced dose dependent reduction in food intake (FIG. 14). Liraglutide, a known appetite suppresion agent, also led to a significant reduction of food intake. These data demonstrate that Perturbagen 6 reduced food intake during a high-fat-diet treatment.

Oral Glucose Tolerance Test

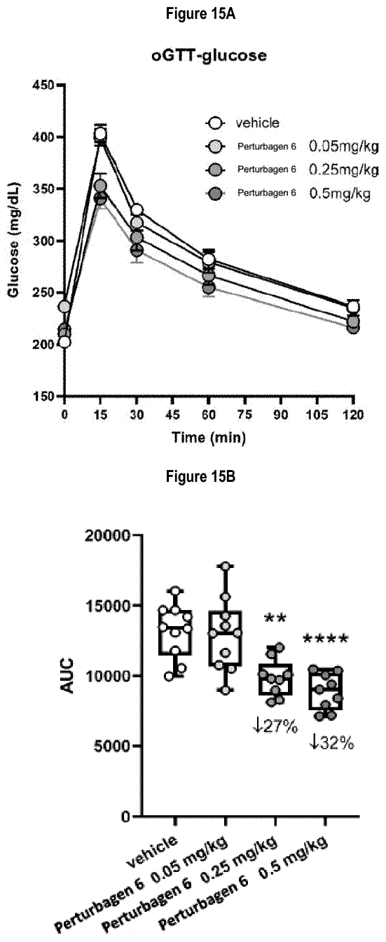

An oral glucose tolerance test was performed after 4 weeks of treatment for vehicle and Perturbagen 6 0.05, 0.25 and 0.5 mg/kg groups. As shown in FIG. 15A-15B, Perturbagen 6 treatment led to a dose dependent reduction in plasma glucose throughout the glucose tolerance test when compared to Vehicle. Perturbagen 6 0.25 and 0.5 mg/kg doses significantly reduced the area under the curve by 27% and 32% respectively. These results demonstrate that Perturbagen 6 dose-dependently improved glycemic control in diet-induced obese mice.

Non-Limiting Conclusions and Interpretations

Perturbagen 6 at doses equivalent (0.5 mg/kg) or lower (0.25 and 0.05 mg/kg) to the lowest clinical dose for psychosis resulted in a significant prevention of weight gain with reduction in percent of weight gain of 9.6%, 5% and 3.3%, respectively, by day 35 of treatment. The highest dose (5 mg/kg) exceeds the clinical dose for psychosis, resulting in possible adverse effects in behavior and reduced food intake. Perturbagen 6 significantly improves the glycemic control at doses that could be safely used for a metabolic-related indication. Obese mice dosed for 5 weeks with 0.5, 0.25 and 0.05 mg/kg of Perturbagen 6 were challenged with an oral GTT. The response to glucose challenge was assessed by measuring the blood glucose levels before and after 15, 30, 60, and 120 min of the glucose administration and calculating the area under the curve (AUC). Mice treated with 0.25 and 0.5 mg/kg Perturbagen 6 showed a significant improvement in glucose handling evidenced by reduction of the AUC by 27% and 32% respectively.

### Example 11

### In Vivo Efficacy: Studies in Obese Pre-Diabetic Non-Human Primates

Study Objective

The objective of these studies is to understand translation of cellular and in vivo rodent efficacy data of Perturbagen 6 and analogues thereof in a higher animal species. The protocol covers procedures required to administer the test article daily (oral) for up to three months, conduct intravenous glucose tolerance tests (ivGTT) and to collect blood samples and subcutaneous fat biopsies in six spontaneously obese, pre-diabetic live animals (Cynomolgus monkeys). The blood samples and subcutaneous fat biopsies will be used to assess clinical chemistry (glucose, insulin resistance levels and lipid profile) and adipose gene signature by single nuclei sequencing of the test article. The expectation will be to observe a significant lowering of hyperinsulinemia and improvement in homeostatic model assessment of insulin resistance (HOMA-IR).

### Example 12

### Induction of White-to Beige Transition of Adipocytes In Vitro and In Vivo Using Small Molecules as a Therapeutic Approach to Obesity

Pharmacological recruitment of thermogenic adipocytes is a long-sought therapeutic intervention for obesity. This Example was aimed at identifying new pharmaceutical beigeing agents by leveraging a machine learning platform. To dissect the white-to-beige transition of adipocytes, single nuclei RNA (snRNA) sequencing data was generated from human white adipocytes transdifferentiated into beige using pioglitazone. In parallel, the snRNA data was analyzed from subcutaneous adipose tissue of mice exposed to cold or treated with beta-3 adrenergic receptor agonist CL-316,243 (see Rajbhandari et al., Elife. 8:e49501 (2019), which is incorporated by reference herein in its entirety). The machine learning platform identified 115 small molecules for their potential to induce several components of the transition of white-to-beige adipocytes. The identified molecules were tested in vitro in human adipocytes for increase of oxygen consumption rate and induction of beigeing transcriptional signature. An in vitro assay validated 22 compounds which could induce the beige cell state in vitro both phenotypically and transcriptionally. Amongst these hits, Perturbagen-1317 (C₂₁H₁₉ClFNO₄S; 435.9 g/mol) was further identified as a potent stimulant of beige state in the diet-induced mouse model of obesity, demonstrating successful in vivo translation of in vitro derived gene signatures. In summary, the machine learning, drug discovery platform identified small molecules that induce the beige state in human white adipocytes in vitro and in mouse adipose tissue in vivo.

The machine learning platform can provide a novel drug discovery paradigm to create medicines designed to change cell behaviors and reverse the course of disease. The machine learning platform and process can be applied to identify small molecules capable of inducing the transdifferentiation of white adipocytes into beige adipocytes.

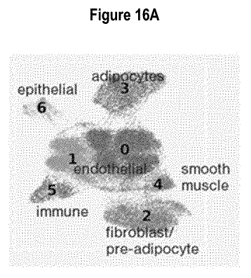

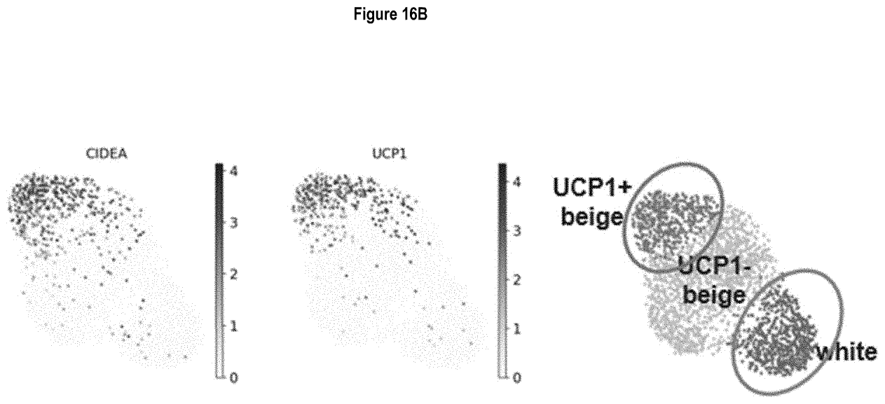

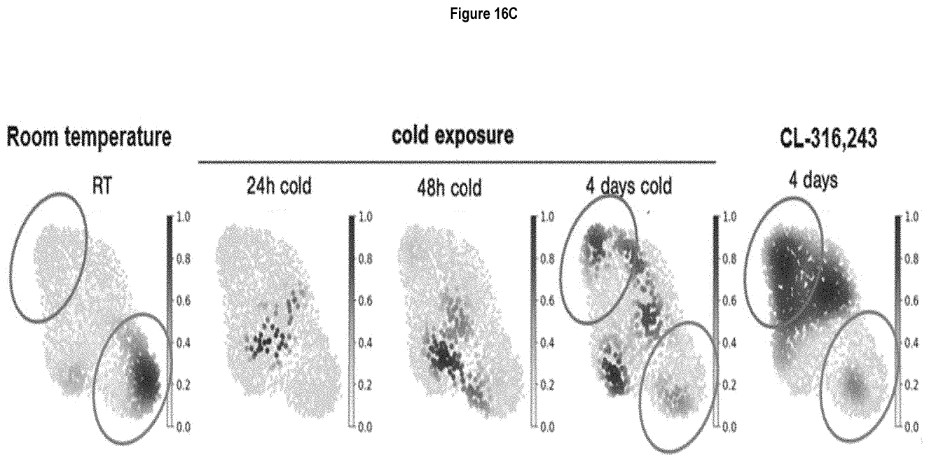

The in vivo dataset was found to capture white and beige adipocyte cell states (FIGS. 16A-16C). Inguinal Adipose tissue from mice was challenged with CL-316,243, a β3-receptor agonist and sequenced at a single cell level (FIG. 16A). Based on the single cell manifold, beige and white adipocytes populations were characterized based on UPC1 expression (FIG. 16B). The dynamic transition of cells states induced by cold and drug treatments was modeled (FIG. 16C).

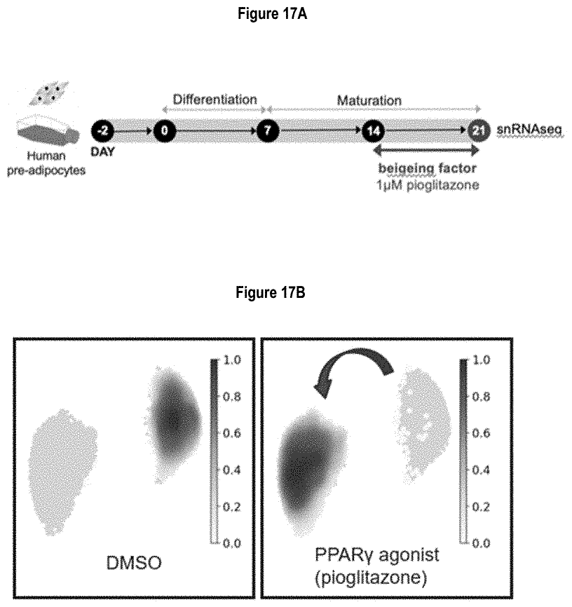

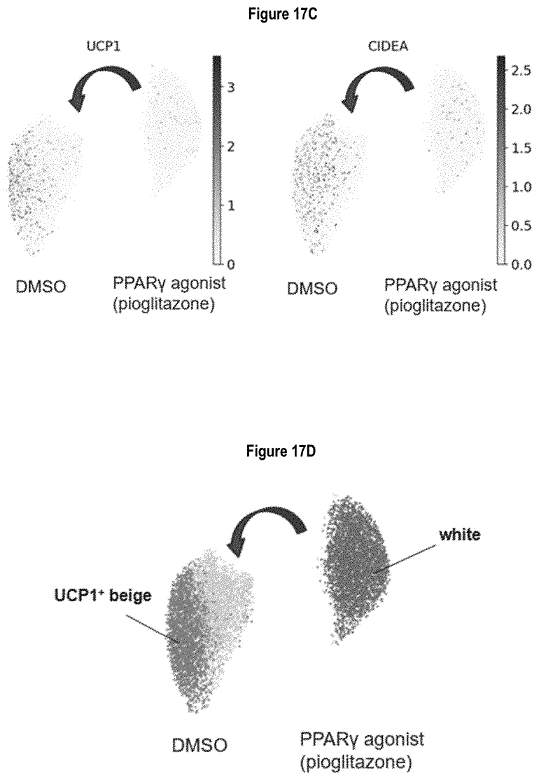

In vitro human adipocytes were treated pioglitazone were found to capture white and beige adipocyte cell states (FIGS. 17A-17D). Human pre-adipocytes were incubated over 21 days and underwent snRNAseq after maturation (FIG. 17A), and then were treated with PPARg agonist (1 μg pioglitazone) between 14-day and 21-day period (FIG. 17A). Change in adipocyte population was induced by pioglitazone treatment (FIG. 17B). Expression levels of UCP1 and CIDEA were analyzed (FIG. 17C). Beige and white adipocytes population was characterized based on UCP1 expression induced by pioglitazone (FIG. 17D).

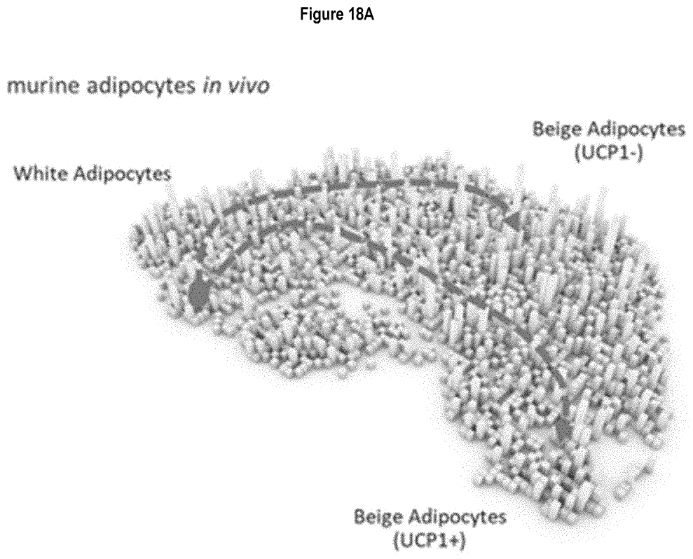

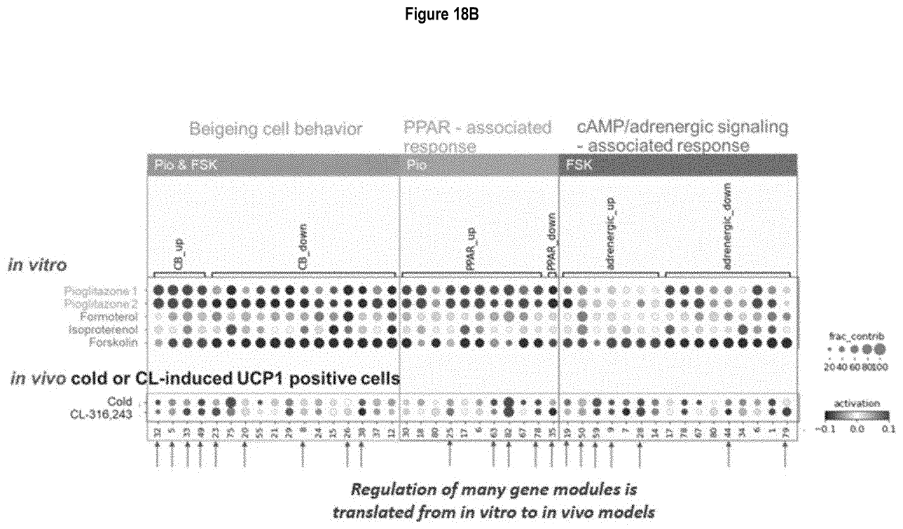

The in silico model of cellular behavior of beige and white adipocytes population characterized based on UPC1 expression induced by pioglitazone was generated (FIG. 18A-18B). Cross functional insight generation was carried out via map integrating disease biology with supporting visualizations (FIG. 18A). Similarities were found in cell behavior modules defining the cell transition between in vitro and in vivo preclinical models (FIG. 18B).

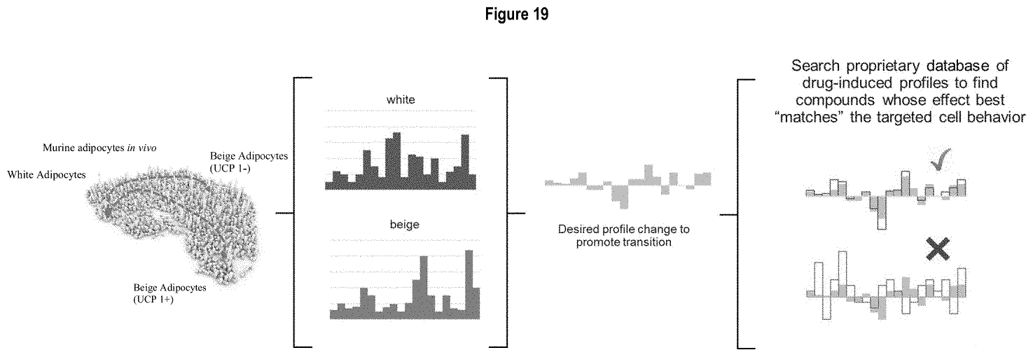

Small molecules likely to induce beigeing of white adipocytes were identified using a machine learning platform (FIG. 19). This machine learning platform can be used to identify new chemistry to induce targeted cell behavior (FIG. 19).

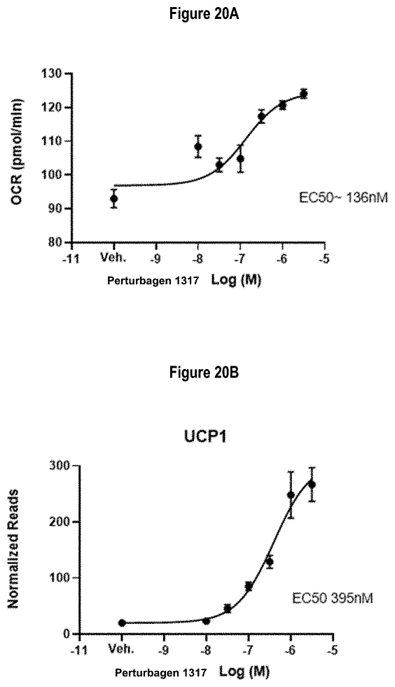

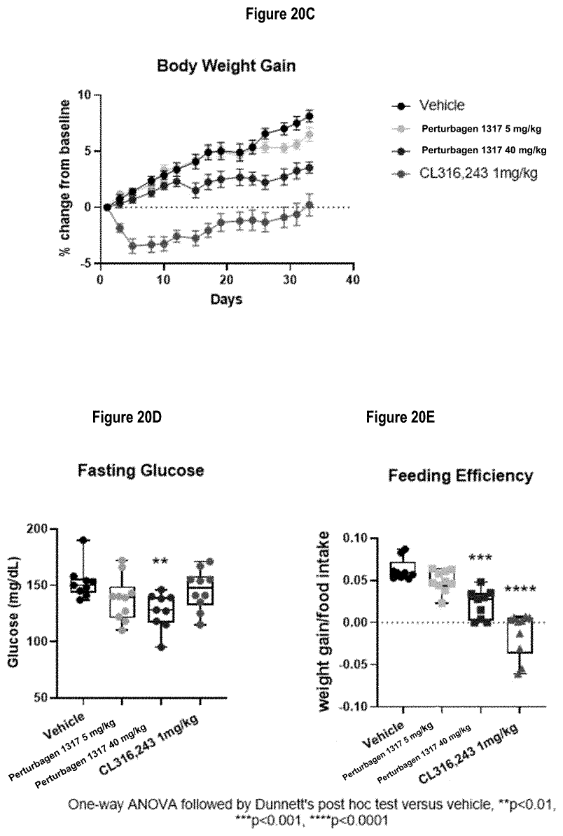

Perturbagen-1317 was identified as a beigeing agent (FIG. 20A-20E). Perturbagen-1317 was found to dose-dependently increase oxygen consumption rate (OCR) in human adipocytes (FIG. 20A). Dose dependent induction of UCP1 gene expression in human adipocytes was graphed (FIG. 20B). Diet-induced obese mice were treated with Perturbagen-1317 at various dosages and results show a reduction in weight gain (FIG. 20C), improved fasting glucose (FIG. 20D). and a reduction in feeding efficiency (FIG. 20E).

Materials and Methods

Single Nuclei Datasets: Inguinal adipose tissue from mice was exposed to cold or treated with beta-adrenergic agonist CL316,243 (see Rajbhandari et al., Elife. 8:e49501 (2019), which is incorporated by reference herein in its entirety).

In vitro human adipocytes were treated with either PPARg agonist (pioglitazone), beta adrenergic agonists (Formoterol, Isoproterenol) or Forskolin.

In vitro human adipocytes: Zenbio human preadipocytes (lot #SL066) differentiated according to manufacturer's instructions

Gene expression analysis method included Single Nuclei Sequencing and Nanostring Technologies.

Cellular Oxygen Consumption: Agilent Seahorse XFe96 Analyzers.

In vivo model: C57BL/6J (Jax #380050) at 16 weeks old after being fed for 10 weeks on 60% kcal HFD (Research Diets #D12492i).

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present technology is not entitled to antedate such publication by virtue of prior disclosure.

As used herein, all headings are simply for organization and are not intended to limit the disclosure in any manner. The content of any individual section may be equally applicable to all sections.

### EQUIVALENTS

While the disclosure has been disclosed in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method for directing a change in cell state of a progenitor cell comprising:
contacting a population of cells comprising a progenitor cell with at least one perturbagen selected from Table 3, or a variant thereof,
wherein the at least one perturbagen is capable of altering a gene signature in the progenitor cell; and
wherein the progenitor cell is an adipocyte stem cell or a mesenchymal stem cell.

2. A method for directing a change in cell state of a progenitor cell, comprising:
contacting a population of cells comprising a progenitor cell with at least one perturbagen capable of altering a gene signature in the progenitor cell,
wherein altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1 and
wherein the progenitor cell is an adipocyte stem cell or a mesenchymal stem cell.

3. A method for directing a change in cell state of a progenitor cell, comprising:
contacting a population of cells comprising a progenitor cell with at least one perturbagen selected from Table 3, or a variant thereof, and capable of altering a gene signature in the progenitor cell;
wherein altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1; and
wherein the progenitor cell is an adipocyte stem cell or a mesenchymal stem cell.

4. The method of any one of claims 1-3, wherein altering the gene signature comprises a change in expression and/or activity of one or more genes in the progenitor cell of a network module designated in the network module column of Table 1.

5. The method of any one of claims 1-4, wherein the change in cell state provides an increase in the number of one or more of adipoblasts, Myf5− progenitor cells, beige preadipocytes, and/or beige adipocytes.

6. The method of claim 5, wherein the increase in the number of adipoblasts, Myf5− progenitor cells, beige preadipocytes, and/or beige adipocytes is relative to the number of adipoblasts, Myf5− progenitor cells, beige preadipocytes, and/or beige adipocytes obtained from a population of progenitor cells that are not contacted with the at least one perturbagen and/or relative to the number of adipoblasts, Myf5− progenitor cells, beige preadipocytes, and/or beige adipocytes obtained from a population of progenitor cells prior to contacting with the at least one perturbagen.

7. The method of any one of claims 1-6, wherein the change in cell state provides an increase in the number of beige adipocytes.

8. The method of claim 7, wherein the increase in the number of beige adipocytes is relative to the number of beige adipocytes obtained from a population of progenitor cells that are not contacted with the at least one perturbagen and/or relative to the number of beige adipocytes obtained from a population of progenitor cells prior to contacting with the at least one perturbagen.

9. The method of any one of claims 1-8, wherein the number of mesenchymal stem cells is decreased.

10. The method of any one of claims 1-8, wherein the number of mesenchymal stem cells is increased.

11. The method of claim 10, wherein the increase in the number of mesenchymal stem cells is due in part to increased cell proliferation of the mesenchymal stem cells.

12. The method of claim 10 or 11, wherein the increase in the number of mesenchymal stem cells is relative to the number of mesenchymal stem cells in a population of progenitor cells that are not contacted with the at least one perturbagen and/or relative to the number of mesenchymal stem cells in a population of progenitor cells prior to contacting with the at least one perturbagen.

13. The method of any one of claims 1-12, wherein the number of beige preadipocytes, and/or beige adipocytes is increased after contacting the population of progenitor cells comprising an adipocyte stem cell or mesenchymal stem cell with the at least one perturbagen.

14. The method of any one of claims 1-13, wherein the at least one perturbagen is selected from Table 3, or a variant thereof, comprises at least 2, at least 3, at least 4, or at least 5 perturbagens selected from Table 3, or variants thereof.

15. The method of any one of claims 2-14, wherein the one or more genes are selected from the genes designated as an “up” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, or 55 genes selected from the genes designated as an “up” gene in the gene directionality column of Table 1.

16. The method of any one of claims 2-15, wherein the one or more genes are selected from MRPL12, MIF, BIRC5, CDK1, UBE2C, CISD1, TOMM70A, RRS1, TRAP1, DLD, GAPDH, CDK4, MRPS16, GALE, CAT, PFKL, CEBPA, SCP2, LPGAT1, NT5DC2, GNB5, LAP3, HSPA4, PPARG, HSPD1, NOLC1, SDHB, PGAM1, TIMM9, CCNB2, IFRD2, MPC2, STMN1, PARP1, UBE2A, GSTZ1, SCARB1, HADH, PEX11A, ETFB, HSD17B10, PXMP2, CIAPIN1, DNAJC15, FKBP4, SMARCA4, BZW2, VDAC1, ISOC1, CYCS, G3BP1, CD320, MYLK, EIF4EBP1, and/or PHGDH.

17. The method of any one of claims 2-16, wherein the one or more genes are selected from the genes designated as an “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, or 73 genes selected from the genes designated as an “down” gene in the gene directionality column of Table 1.

18. The method of any one of claims 2-17, wherein the one or more genes are selected from JUN, ID2, ZFP36, BAMBI, HERPUD1, NCK1, MYC, SQSTM1, NFKBIA, IER3, TIPARP, HES1, CYB561, HSD17B11, FYN, PLSCR1, CIRBP, FOS, NUCB2, S100A13, RSU1, ASAH1, TIMP2, COL1A1, MYL9, LOXL1, COL4A1, DNM1, MMP2, PTGS2, SSBP2, RTN2, VAT1, GAA, PROS1, B4GAT1, PNP, DRAP1, PRSS23, IGFBP3, TPM1, ILK, SCRN1, FHL2, KDM5B, GADD45B, EGFR, GRN, SERPINE1, TMEM50A, CLTB, GNA12, FKBP14, CALU, STXBP1, HTRA1, UGDH, PLA2G4A, EXT1, CTSL, NENF, TGFBR2, EGR1, P4HA2, GNAS, APP, HSPA1A, ITGB5, EPB41L2, GSTM2, ICAM1, CEBPD, and/or CHIC2.

19. The method of any one of claims 1-18, wherein the contacting the population of progenitor cells occurs in vitro or ex vivo and/or in vivo in a subject.

20. The method of any one of claims 1-19, wherein the method increases or stimulates the beiging and/or browning of white adipose tissue (WAT), optionally as compared to the beiging and/or browning of WAT in the absence of a perturbagen and/or prior to contacting with the at least one perturbagen.

21. A perturbagen for use in the method of any one of claims 1-20.

22. A method for promoting the formation of a beige adipocyte, or an immediate progenitor thereof, comprising:
exposing a starting population of stem/progenitor cells comprising a non-lineage committed mesenchymal stem cell nor adipocyte stem cell to a perturbation having a perturbation signature that promotes the transition of the starting population of stem/progenitor cells into a beige adipocyte,
wherein the perturbation signature comprises increased expression and/or activity of one or more of genes selected from the genes designated as an “up” gene in the gene directionality column of Table 1 and/or a decreased expression and/or activity in the non-lineage committed adipocyte stem cell or mesenchymal stem cell of one or more genes selected from the genes designated as an “down” gene in the gene directionality column of Table 1.

23. The method of claim 22, wherein the perturbation signature comprises an increase in expression and/or activity of one or more genes in the progenitor cell of an activation of a network module designated in the network module column of Table 1.

24. A method of increasing a quantity of beige adipocytes, or immediate progenitors thereof, comprising:
exposing a starting population of stem/progenitor cells comprising a non-lineage committed adipocyte stem cell or mesenchymal stem cell to a pharmaceutical composition that promotes the formation of lineage specific progenitor population selected from adipoblasts, Myf5− progenitor cells, beige preadipocytes, and/or beige adipocytes, the pharmaceutical composition promoting the transition of a primitive stem/progenitor population into the lineage specific progenitor population that has the capacity to differentiate into beige preadipocytes, and/or beige adipocytes or immediate progenitors thereof,
wherein the pharmaceutical composition comprises at least one perturbagen selected from Table 3, or a variant thereof.

25. A method for treating a disease or disorder characterized by an abnormal number of beige adipocytes, white adipocytes, beige preadipocytes, and/or white preadipocytes, comprising:
(a) administering to a patient in need thereof a therapeutically effective amount of at least one perturbagen selected from Table 3, or a variant thereof, wherein the at least one perturbagen is capable of changing a gene signature in a progenitor cell; or
(b) administering to a patient in need thereof a cell, the cell having been contacted with at least one perturbagen selected from Table 3, or a variant thereof, wherein the at least one perturbagen is capable of changing a gene signature in a progenitor cell.

26. A method for treating a disease or disorder characterized by an increased number of white adipocytes, and/or white preadipocytes, comprising:
(a) administering to a patient in need thereof a therapeutically effective amount of at least one perturbagen selected from Table 3, or a variant thereof, wherein the at least one perturbagen is capable of changing a gene signature in a progenitor cell; or
(b) administering to a patient in need thereof a cell, the cell having been contacted with at least one perturbagen selected from Table 3, or a variant thereof, wherein the at least one perturbagen is capable of changing a gene signature in a progenitor cell.

27. A method for treating a disease or disorder characterized by a decreased number of beige adipocytes, and/or beige preadipocytes, comprising:
(a) administering to a patient in need thereof a therapeutically effective amount of at least one perturbagen selected from Table 3, or a variant thereof, wherein the at least one perturbagen is capable of changing a gene signature in a progenitor cell; or
(b) administering to a patient in need thereof a cell, the cell having been contacted with at least one perturbagen selected from Table 3, or a variant thereof, wherein the at least one perturbagen is capable of changing a gene signature in a progenitor cell.

28. The method of any one of claims 25-27, wherein the disease or disorder is obesity, morbid obesity, morbid obesity prior to surgery, obesity linked inflammation, obesity linked gallbladder disease, obesity induced sleep apnea, hyperlipidemia, dyslipidemia, hypercholesterolemia, atherogenic dyslipidemia, fatty liver disease (FLD), nonalcoholic fatty liver disease (NAFLD), Non-Alcoholic Steatohepatitis (NASH), Prader-Willi Syndrome, insulin resistance, glucose intolerance, pre-diabetes, increased fasting glucose, diabetes mellitus, type 2 diabetes mellitus, inflammation, vascular inflammation, hypertension, endothelial dysfunction, atherosclerosis, arteriosclerosis, coronary heart disease, peripheral artery disease, stroke, or microvascular disease, arterial remodelling, cardiovascular disease (CVD), atherosclerotic cardiovascular disease, metabolic syndrome or a combination of any two or more thereof. obesity, hyperlipidemia, NAFLD, Type II Diabetes, inflammation, hypertension, cardiovascular disease, a metabolic disorder, excess body weight, atheromatous disease, β-cell dysfunction, heart disease, hyperglycemia, impaired glucose tolerance, an inflammatory disorder, latent autoimmune diabetes (LADA), nephropathy, neuropathy, retinopathy, Syndrome X, and/or type 1 diabetes.

29. The method of any one of claims 25-28, wherein the method alleviates one or more symptoms selected from the group of abdominal obesity, a body mass index (BMI) of 30 or higher, a blood triglyceride level of 150 or higher, a blood HDL level of less than 40 or higher in men, a blood HDL level of 50 or higher in women, a blood pressure of 130/85 or higher, a fasting blood sugar of 110 or higher, hyperlipidemia, dyslipidemia, hypercholesterolemia, atherogenic dyslipidemia insulin resistance, glucose intolerance, pre-diabetes, increased fasting glucose, diabetes mellitus, and a combination of any two or more thereof.

30. A method for selecting a subject for method of any one of claims 25-29, comprising:
obtaining from a subject having the disease or disorder a sample of cells comprising a non-lineage committed adipocyte stem cell or mesenchymal stem cell; and
contacting the sample of cells with least one perturbagen selected from Table 3 or a variant thereof,
wherein the at least one perturbagen alters a gene signature in the sample of cells, the subject is selected as a patient.

31. A method for selecting a subject for method of any one of claims 25-29, comprising:
obtaining from a subject having the disease or disorder a sample of cells comprising a non-lineage committed adipocyte stem cell or mesenchymal stem cell; and
contacting the sample of cells with at least one perturbagen capable of altering a gene signature in a non-lineage committed adipocyte stem cell or mesenchymal stem cell,
wherein the at least one perturbagen increases in the sample of cells the expression and/or activity of one or more genes selected from the genes designated as an “up” gene in the gene directionality column of Table 1, and/or decreases in the sample of cells the expression and/or activity of one or more genes selected from the genes designated as an “down” gene in the gene directionality column of Table 1, the subject is selected as a patient.

32. A method for selecting a subject for method of any one of claims 25-31, comprising:
obtaining from a subject having the disease or disorder a sample of cells comprising a non-lineage committed adipocyte stem cell or mesenchymal stem cell; and
contacting the sample of cells with least one perturbagen selected from Table 3 or a variant thereof,
wherein the at least one perturbagen increases in the sample of cells the expression and/or activity of one or more genes selected from the genes designated as an “up” gene in the gene directionality column of Table 1, and/or decreases in the sample of cells the expression and/or activity of one or more genes selected from the genes designated as an “down” gene in the gene directionality column of Table 1, the subject is selected as a patient.

33. The method of any one of claims 27-32, wherein the perturbation signature comprises an increase in expression and/or activity of one or more genes in the progenitor cell of an activation of a network module designated in the network module column of Table 1.

34. The method of claim 33, wherein the one or more genes are selected from MRPL12, MIF, BIRC5, CDK1, UBE2C, CISD1, TOMM70A, RRS1, TRAP1, DLD, GAPDH, CDK4, MRPS16, GALE, CAT, PFKL, CEBPA, SCP2, LPGAT1, NT5DC2, GNB5, LAP3, HSPA4, PPARG, HSPD1, NOLC1, SDHB, PGAM1, TIMM9, CCNB2, IFRD2, MPC2, STMN1, PARP1, UBE2A, GSTZ1, SCARB1, HADH, PEX11A, ETFB, HSD17B10, PXMP2, CIAPIN1, DNAJC15, FKBP4, SMARCA4, BZW2, VDAC1, ISOC1, CYCS, G3BP1, CD320, MYLK, EIF4EBP1, and/or PHGDH.

35. The method of any one of claims 27-32, wherein the one or more genes are selected from the genes designated as an “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, or 55 genes selected from the genes designated as an “down” gene in the gene directionality column of Table 1.

36. The method of claim 35, wherein the one or more genes are selected from JUN, ID2, ZFP36, BAMBI, HERPUD1, NCK1, MYC, SQSTM1, NFKBIA, IER3, TIPARP, HES1, CYB561, HSD17B11, FYN, PLSCR1, CIRBP, FOS, NUCB2, S100A13, RSU1, ASAH1, TIMP2, COL1A1, MYL9, LOXL1, COL4A1, DNM1, MMP2, PTGS2, SSBP2, RTN2, VAT1, GAA, PROS1, B4GAT1, PNP, DRAP1, PRSS23, IGFBP3, TPM1, ILK, SCRN1, FHL2, KDM5B, GADD45B, EGFR, GRN, SERPINE1, TMEM50A, CLTB, GNAI2, FKBP14, CALU, STXBP1, HTRA1, UGDH, PLA2G4A, EXT1, CTSL, NENF, TGFBR2, EGR1, P4HA2, GNAS, APP, HSPA1A, ITGB5, EPB41L2, GSTM2, ICAM1, CEBPD, and/or CHIC2.

37. The method of any one of claims 27-36, wherein the at least one perturbagen is selected from Table 3, or a variant thereof, comprises at least 2, at least 3, at least 4, or at least 5 perturbagens selected from Table 3, or variants thereof.

38. A method of identifying a candidate perturbation for promoting the transition of a starting population of progenitor cells comprising an adipocyte stem cell or a mesenchymal stem cell into beige adipocytes or immediate progenitors thereof, the method comprising:
exposing the starting population of progenitor cells to a perturbation;
identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into beige adipocytes or immediate progenitors thereof following exposure of the population of cells to the perturbation; and
identifying the perturbation as a candidate perturbation for promoting the transition of a population of progenitor cells into beige adipocytes or immediate progenitors thereof based on the perturbation signature,
wherein the perturbation signature is an increase in expression and/or activity in the progenitor cell of one or more genes selected from the genes designated as an “up” gene in the gene directionality column of Table 1, and/or a decrease in expression and/or activity in the progenitor cell of one or more genes selected from the genes designated as an “down” gene in the gene directionality column of Table 1.

39. The method of claim 38, wherein the perturbation signature is an increase in expression and/or activity in the progenitor cell of an activation of a network module designated in the network module column of Table 1.

40. A method for making a therapeutic agent for a disease or disorder selected from obesity, hyperlipidemia, NAFLD, Type II Diabetes, inflammation, hypertension, and/or cardiovascular disease, comprising:
(a) identifying a candidate perturbation according to the method of claim 38 or 39 and
(b) formulating the candidate perturbation as a therapeutic agent for the treatment of the disease or disorder.

41. A method for directing a change in cell state of a progenitor cell, comprising:
contacting a population of cells comprising a progenitor cell with at least one perturbagen capable of altering a gene signature in the progenitor cell,
wherein altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as an “down” gene in the gene directionality column of Table 1,
wherein the progenitor cell is an adipocyte stem cell or a mesenchymal stem cell; and
the change in cell state provides an increase in the number of one or more of adipoblasts, Myf5− progenitor cells, beige preadipocytes, and/or beige adipocytes.

42. The method of claim 41, wherein the increase in the number of adipoblasts, Myf5− progenitor cells, beige preadipocytes, and/or beige adipocytes is relative to the number of adipoblasts, Myf5− progenitor cells, beige preadipocytes, and/or beige adipocytes obtained from a population of progenitor cells that are not contacted with the at least one perturbagen

43. The method of claim 41, wherein the increase in the number of adipoblasts, Myf5− progenitor cells, beige preadipocytes, and/or beige adipocytes is relative to the number of adipoblasts, Myf5− progenitor cells, beige preadipocytes, and/or beige adipocytes obtained from a population of progenitor cells prior to contacting with the at least one perturbagen

44. The method of claim 41, wherein the at least one perturbagen is selected from Table 3, or a variant thereof.

45. The method of claim 44, wherein the at least one perturbagen comprises at least 2, at least 3, at least 4, or at least 5 perturbagens selected from Table 3, or variants thereof.

46. The method of claim 41, the method provides an increase in expression and/or activity of uncoupling protein 1 (UCP1).

47. The method of claim 41, wherein the change in cell state provides an increase in the number of beige adipocytes.

48. The method of claim 41, wherein the one or more genes are selected from MRPL12, MIF, BIRC5, CDK1, UBE2C, CISD1, TOMM70A, RRS1, TRAP1, DLD, GAPDH, CDK4, MRPS16, GALE, CAT, PFKL, CEBPA, SCP2, LPGAT1, NT5DC2, GNB5, LAP3, HSPA4, PPARG, HSPD1, NOLC1, SDHB, PGAM1, TIMM9, CCNB2, IFRD2, MPC2, STMN1, PARP1, UBE2A, GSTZ1, SCARB1, HADH, PEX11A, ETFB, HSD17B10, PXMP2, CIAPIN1, DNAJC15, FKBP4, SMARCA4, BZW2, VDAC1, ISOC1, CYCS, G3BP1, CD320, MYLK, EIF4EBP1, and/or PHGDH.

49. The method of claim 41, wherein the one or more genes are selected from JUN, ID2, ZFP36, BAMBI, HERPUD1, NCK1, MYC, SQSTM1, NFKBIA, IER3, TIPARP, HES1, CYB561, HSD17B11, FYN, PLSCR1, CIRBP, FOS, NUCB2, S100A13, RSU1, ASAH1, TIMP2, COL1A1, MYL9, LOXL1, COL4A1, DNM1, MMP2, PTGS2, SSBP2, RTN2, VAT1, GAA, PROS1, B4GAT1, PNP, DRAP1, PRSS23, IGFBP3, TPM1, ILK, SCRN1, FHL2, KDM5B, GADD45B, EGFR, GRN, SERPINE1, TMEM50A, CLTB, GNAI2, FKBP14, CALU, STXBP1, HTRA1, UGDH, PLA2G4A, EXT1, CTSL, NENF, TGFBR2, EGR1, P4HA2, GNAS, APP, HSPA1A, ITGB5, EPB41L2, GSTM2, ICAM1, CEBPD, and/or CHIC2.
